(19) United States
(12) Patent Application Publication
(10) Pub. No.: US 2022/0370586 A1
(43) Pub. Date: Nov. 24, 2022

(54) METHOD OF GENERATING ACTIVATED T CELLS FOR CANCER THERAPY

(71) Applicant: Cedars-Sinai Medical Center, Los Angeles, CA (US)

(72) Inventors: John Sun YU, Los Angeles, CA (US); Keith L. Black, Los Angeles, CA (US)

(73) Assignee: Cedars-Sinai Medical Center, Los Angeles, CA (US)

(21) Appl. No.: 17/633,505

(22) PCT Filed: Aug. 10, 2020
(86) PCT No.: PCT/US2020/045570

§ 371 Date: Feb. 7, 2022

Related U.S. Application Data
(60) Provisional application No. 62/884,570, filed on Aug. 8, 2019.

(51) Int. Cl. A61K 39/00; A61K 31/341; A61K 31/15; A61K 31/165

(52) U.S. Cl. CPC A61K 39/001186; A61K 39/001188; A61K 39/001178; A61K 39/001122; A61K 39/001106; A61K 39/00119; A61K 39/001119; A61K 31/341; A61K 31/15; A61K 31/165; A61K 39/001192; A61K 2039/5158; A61K 2039/80

(57) ABSTRACT

Described herein are compositions and methods for treating cancer and autoimmune diseases.

### TECHNICAL FIELD

The present disclosure relates generally to compositions and methods for treating cancer. The present invention is directed to as a method to generate T cells that target cancer stem cells that can be carried out outside of the confines of the immunosuppressive milieu using the inventive technique described herein. The present disclosure also relates to treating inflammatory diseases such as autoimmune diseases.

### BACKGROUND

All publications herein are incorporated by reference to the same extent as if each individual publication or patent application was specifically and individually indicated to be incorporated by reference. The following description includes information that may be useful in understanding the present invention. It is not an admission that any of the information provided herein is prior art or relevant to the presently claimed invention, or that any publication specifically or implicitly referenced is prior art.

In addition to chemotherapies, advancements in targeted antigen adjuvant therapies and immunotherapies have shown progress in inducing tumor immunogenicity. Prior work with autologous dendritic cell (DC) therapies pulsed with known tumor associated antigens or tumor lysate showed safety and hints at efficacy in treating cancer including glioblastoma. Pulsing dendritic cells with patient tumor lysate offers the advantage of a unique patient regimen of glioma specific antigens. This strategy can be beneficial since high grade gliomas are typically non-homogenous, adding to the difficulty of treatment and causing eventual relapse. A prior phase II trial for GBM showed an expansion of CD8+ T-cells and cytotoxic T-lymphocytes (CTL) against tumor associated antigens such as MAGE-1, gp100, and HER-2 in 4/9 patients and systemic cytotoxicity response of PBMC in 6/10 patients when dendritic cells were pulsed with tumor-lysate. These therapies attempt to remove antigen presentation outside of the realm of tumor immunosuppression, however T cell activation and expansion remain under the influence of the immunosuppression from the cancer and from radiation and chemotherapies.

Phuphanich, S., et al., Phase I trial of a multi-epitope-pulsed dendritic cell vaccine for patients with newly diagnosed glioblastoma. Cancer Immunol Immunother, 2013. 62(1): p. 125-35.

Yu, J. S., et al., Vaccination with tumor lysate-pulsed dendritic cells elicits antigen-specific, cytotoxic T-cells in patients with malignant glioma. Cancer Res, 2004. 64(14): p. 4973-9.

Yu, J. S., et al., Vaccination of malignant glioma patients with peptide-pulsed dendritic cells elicits systemic cytotoxicity and intracranial T-cell infiltration. Cancer Res, 2001. 61(3): p. 842-7.

Wheeler, C. J., et al., Vaccination elicits correlated immune and clinical responses in glioblastoma multiforme patients. Cancer Res, 2008. 68(14): p. 5955-64.

Regulatory T cells (Treg cells) express high levels of the glucocorticoid-induced tumor necrosis factor-related receptor (GITR), while resting T cells express low levels that are increased upon activation. Modulation of GITR/GITR-Ligand (GITRL) interactions results in enhancement of immune responses. There is a need in the art for agents that modulate GITR/GITRL so as to treat cancer or autoimmune diseases.

GITR/GITRL is a member of Tumor necrosis factor receptor superfamily (TNFRSF), TNFRSF18. It is also referred as Activation-Inducible TNFRSF (AITR).

Cancer immunotherapy is a new tool in the fight against cancer progression. While immune suppression at the tumor site is contributed by various stromal cells such macrophages, cancer-associated fibroblasts, checkpoint mediated T-cell suppression has been identified as potential therapeutic targets. Checkpoint molecules are PD-1, OX40, CTLA-4 and GITR. Currently, antibody-based therapeutics targeted these checkpoint molecules are used in clinic except molecules targeting GITR, a major regulator of Foxp3+T regulatory (Treg) cells.

Glucocorticoid-induced TNR family related protein Ligand (GITRL) is a T-cell cytokine that co-stimulates Teffector (Teff) cells through GITR receptor and neutralizes suppressive activity of T regulatory (Treg) cells and seems to inhibit Foxp3 expression.

Due its central role in regulating Treg, GITR receptor complex is considered an optimal therapeutic target for treating autoimmunity and cancer. Indeed, recently, an anti-GITR antibody, MK-4166 has been shown to eradicate established melanoma and colon tumors in preclinical mouse models (Mahne et al. 2017).

As co stimulatory cytokines. GITR receptor and its ligand belong to the TNF/TNFR super family, which has been extensively studied. GITR is constitutively expressed at high levels on CD4+CD25+ regulatory T cell and activated T cells. GITR ligand (GITRL) is constitutively expressed on antigen-presenting cells. Signaling through GITR, can either boost Treg suppression or reduce Treg suppression leading to either diminished T-effector cells or enhanced ability of T effector cells to recognize and respond to self-antigens, for example cancer/tumor cells. Pharmacological manipulation of GITR signaling may have potential application for anti-tumor treatment and autoimmunity.

### SUMMARY

The following embodiments and aspects thereof are described and illustrated in conjunction with systems, composition and methods which are meant to be exemplary and illustrative, not limiting in scope.

The present invention is directed to a method for treating cancer in a subject in need thereof comprising administering to the subject a therapeutically effective amount of T cells that have been activated ex vivo with an antigen presenting cell. The present invention is also directed to a method for treating cancer in a subject in need thereof comprising administering to the subject a therapeutically effective amount of a sample of T-eff cells that have been activated ex vivo, and enriched or expanded, wherein the T-eff cells are enriched or expanded by contacting the T-eff cells with a GITR/GITRL agonist with or without the presence of T-reg cells. And the present invention is also directed to a method of providing activated T cells, comprising activating T-cells by contacting ex vivo T cells with antigen bearing antigen presenting cells, and enriching or expanding T-eff cells comprising contacting T-eff cells with a GITR/GITRL agonist with or without the presence of T-reg cells.

In the above, the antigen presenting cell may be dendritic cell. The antigen may be cancer stem cell antigen. The antigen may be one that is expressed in tumors, such as glioblastoma tumors or neural crest cell derived tissue tumor. In one aspect, the antigen may be a polypeptide of gp100, MAGE1, NY-ESO-1, TRP-2, EphA2, AIM2, HER2/neu, IL-13Ra2, or MAGE-A1, or a combination thereof. The polypeptide may be about 8 to about 20 amino acids long, more preferably, about 8 to about 13 amino acids long, wherein the polypeptide is an epitope for activation of T cells. In one aspect, the polypeptide may be for gp100, the polypeptide may be IMDQVPFSV (SEQ ID NO:6); for MAGE1, the polypeptide may be EADPTGHSY (SEQ ID NO:7); for NY-ESO-1, the polypeptide may be SLLMWITQC (SEQ ID NO:8); for TRP-2, the polypeptide may be SVYDFFVWL (SEQ ID NO:9); for EphA2, the polypeptide may be TLADFDPRV (SEQ ID NO:10); for AIM2, the polypeptide may be RSDSGQQARY (SEQ ID NO:11); for HER2/neu, the polypeptide may be VMAGVGSPYV (SEQ ID NO:12); for IL-13Ra2, the polypeptide may be WLPFGFIL (SEQ ID NO:13); or for MAGE-A1, the polypeptide may be KVLEYVIKV (SEQ ID NO:14).

In the above methods, helper antigen may be used. The helper antigen may be a polypeptide of antigen gp100, NY-ESO-1, TRP-2, EphA2, HER2/neu, or MAGE-A1, or a combination thereof. The polypeptide may be about 8 to about 30 amino acids, preferably 8 to about 20, or about 8 to about 12 amino acids, wherein the polypeptide is an epitope for activation of T cells. In one aspect, the polypeptide may be for gp100, the polypeptide may be SLAVVSTQLIMPGQE (SEQ ID NO:15); for NY-ESO-1, the polypeptide may be PGVLLKEFTVSGNILTIRLTAADHR (SEQ ID NO:16); for TRP-2, the polypeptide may be QCTEVRADTRPWSGP (SEQ ID NO:17) or KKRVHPDYVITTQHWL (SEQ ID NO:18); for EphA2, the polypeptide may be EAGIMGQFSHHNIIR (SEQ ID NO:19); or for HER2/neu, the polypeptide may be KVPIKWMALESILRRRF (SEQ ID NO:20), KIFGSLAFLPESFDGDPA (SEQ ID NO:21), RRLLQETELVEPLTPS (SEQ ID NO:22), or ELVSEFSRMARDPQ (SEQ ID NO:23).

The method may comprise contacting the T cells with GITR/GITRL agonist of Formula I. The GITR/GITRL agonist may be represented by a peptide having the sequence set forth in SEQ ID NO:1 or 2 or a variant, derivative or functional equivalent thereof. The present methods may further comprise administering existing therapies for cancer to the subject either co-administered or sequentially. The cancer may be T-cell/B-cell lymphomas (Hodgkin's lymphomas and/or non-Hodgkins lymphomas), brain tumor, breast cancer, colon cancer, lung cancer, hepatocellular cancer, gastric cancer, pancreatic cancer, cervical cancer, ovarian cancer, liver cancer, bladder cancer, cancer of the urinary tract, thyroid cancer, renal cancer, carcinoma, skin cancer, head and neck cancer, brain cancer, and prostate cancer, androgen-dependent prostate cancer and androgen-independent prostate cancer. The mentioned T-eff or T-reg cells may be autologous or allogeneic relative to the subject. The T-eff and T-reg cells may be present in a starting ratio of about 1:1.

GITR/GITRL agonist mainly decreases Treg numbers and function. However, it also has a proliferative effect on T-eff cells, both helper CD4+ cells and cytotoxic CD8+ T cells. The GITR agonist can boost activated T cells through its positive effect on both the CD8+ cytotoxic and CD4+ helper T cells and its negative effect on Treg cells. GITR/GITRL may be used with activated T cells, including activated cytotoxic T lymphocytes.

In another aspect, the invention is directed to a method for treating an inflammatory disease in a subject in need thereof comprising administering to the subject T cells that have been activated ex vivo with an antigen presenting cell bearing inflammatory disease specific antigen. In another aspect, the invention is directed to a method for treating inflammatory disease in a subject in need thereof comprising administering to the subject a therapeutically effective amount of T cells that have been activated ex vivo, and GITR/GITRL antagonist either in vivo or engineered T cells that have been enriched or expanded for T-reg in vivo or ex vivo, wherein the T-reg cells are enriched or expanded and T-eff cells are modified by contacting the T-eff cells with a GITR/GITRL antagonist with or without the presence of T-reg cells. In another aspect, the invention is directed to a method of enriching or expanding activated T cells and T-reg cells comprising contacting T cells with a GITR/GITRL antagonist with or without the presence of T-eff cells.

The antigen presenting cell may be dendritic cell. The antigen presenting cell may bear inflammatory disease specific antigen. The antigen may be autoimmune disease specific peptide. The method may further comprise contacting the activated T cells ex vivo with GITR/GITRL antagonist such as compound of Formula II. Inflammatory disease may be autoimmune disease. The method may include administering existing therapies for inflammatory disease to the subject either co-administered or sequentially. The autoimmune disease may be rheumatoid arthritis, osteoarthritis, asthma, dermatitis, psoriasis, cystic fibrosis, post transplantation late and chronic solid organ rejection, multiple sclerosis, systemic lupus erythematosus, Sjogren's syndrome, Hashimoto thyroiditis, polymyositis, scleroderma, Addison disease, vitiligo, pernicious anemia, glomerulonephritis and pulmonary fibrosis, inflammatory bowel diseases, autoimmune diabetes, diabetic retinopathy, rhinitis, ischemia-reperfusion injury, post-angioplasty restenosis, chronic obstructive pulmonary diseases (COPD), Grave's disease, gastrointestinal allergies, conjunctivitis, atherosclerosis, coronary artery disease, angina, cancer metastasis, small artery disease, graft-versus-host disease, or mitochondrial related syndrome. The T-eff or T-reg cells may be autologous or allogeneic relative to the subject. The T-eff and T-reg cells may be present in a starting ratio of about 1:1.

This present invention removes the antigen presentation process as well as T cell activation and expansion out of the immunosuppressive confines of the cancer patient. It enables the expansion of cytotoxic T cells using cytotoxic T cell antigens and helper antigens of cancer stem cells. These antigens can be derived from cancer stem cells and remain undefined or may use known cancer stem cell associated antigens as well as helper antigens that are defined and used to expand both cytotoxic and helper T cell antigens.

In one aspect, the invention is directed to generating T cells in vitro with certain cytokines in sequence and then priming these T cells with dendritic cells loaded with certain tumor-specific epitopes, from both cytotoxic and/or helper antigens. This method of activation and priming generates potent antigen specific T cells that can recognize and kill cancer stem cells. Administering these activated T cells into patients suffering from cancer would be expected to kill the cancer stem cells that propagate the patient's tumor, thereby achieving a therapeutic response.

The cancer may be glioblastoma, and in particular intracranial glioblastoma. The administering may be carried out intravenously.

In this regard, activated T cells specifically kill cancer stem cells. The inventive activated T cells do not generate autoimmune responses to normal stem cells. And the activated T cells localize in the area of specificity, in particular intracranial tumor and invoke tumor responses.

Also provided herein is a compound that can be used together with the T cells generated using the above methods to activate and expand T eff:

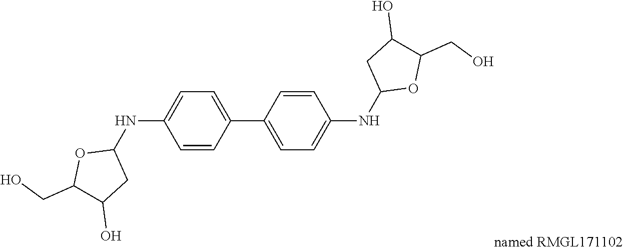 [SMILES: OCC1OC(Nc2ccc(-c3ccc(NC4CC(O)C(CO)O4)cc3)cc2)CC1O]

Also provided herein is a compound of Formula (I):

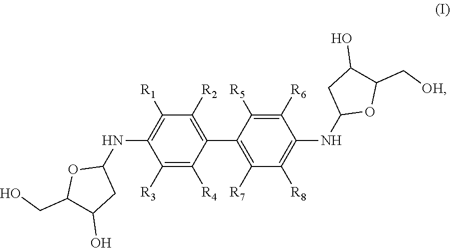

or a pharmaceutically acceptable salt, ester or prodrug thereof;
wherein:

R₁ is hydrogen or an optionally substituted substituent;

R₂ is hydrogen or an optionally substituted substituent;

R₃ is hydrogen or an optionally substituted substituent;

R₄ is hydrogen or an optionally substituted substituent;

R₅ is hydrogen or an optionally substituted substituent;

R₆ is hydrogen or an optionally substituted substituent;

R₇ is hydrogen or an optionally substituted substituent; and

R₈ is hydrogen or an optionally substituted substituent;

wherein optionally any two or more of R₁, R₂, R₃, R₄, R₅, R₆, R₇, or R₈ may be joined together to form one or more rings.

Also provided herein are GITR agonists selected from any one or more of the compounds having the structure described in Formula I that may be used with the methods described above.

Also provided herein are GITR agonists selected from any one or more or all of SEQ ID NO: 1, and/or SEQ ID NO: 2, or a variant, derivative or functional equivalent thereof that may be used with the methods described above.

Further provided herein are compositions comprising GITR agonists described herein. Also provided are methods for using the GITR agonists for treating cancer in a subject by administering a therapeutically effective amount of the compositions comprising GITR agonists. In some embodiments, the methods further comprise administering existing therapies for cancer to the subject. In various embodiments, the compositions comprising the GITR agonists and the existing therapies are co-administered or administered sequentially.

In one aspect, the present invention is directed to a method for treating cancer in a subject in need thereof comprising administering to the subject a therapeutically effective amount of a GITR/GITRL agonist as described herein together with the activated T cells generated ex vivo using the methods described above.

The activated T cells may be co-administered with existing therapies for cancer to the subject or sequentially administered. The cancer may be T-cell/B-cell lymphomas (Hodgkin's lymphomas and/or non-Hodgkins lymphomas), brain tumor, breast cancer, colon cancer, lung cancer, hepatocellular cancer, gastric cancer, pancreatic cancer, cervical cancer, ovarian cancer, liver cancer, bladder cancer, cancer of the urinary tract, thyroid cancer, renal cancer, carcinoma, skin cancer such as melanoma, head and neck cancer, brain cancer, and prostate cancer, androgen-dependent prostate cancer or androgen-independent prostate cancer.

In another aspect, the invention is directed to treating cancer in a subject in need thereof comprising administering to the subject a therapeutically effective amount of a sample of T-eff cells that have been activated, enriched or expanded, wherein the T-eff cells are enriched or expanded by contacting the T-eff cells with a GITR/GITRL agonist described herein with or without the presence of T-reg cells, and further activation is carried out by exposing T cells to antigen presenting cells ex vivo. The T-eff or T-reg cells may be autologous or allogeneic relative to the subject.

In another aspect, the invention is directed to a method of enriching or expanding T-eff cells comprising contacting T-eff cells with a GITR/GITRL agonist described herein with or without the presence of T-reg cells. Preferably, T-reg cells are present. The T-eff and T-reg cells may be present in a starting ratio of about 1:1.

Also provided herein is a compound of Formula (I):

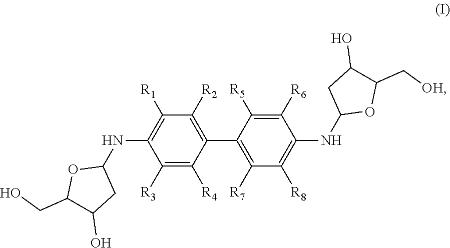

or a pharmaceutically acceptable salt, ester or prodrug thereof;
wherein:

R₁ is hydrogen or an optionally substituted substituent;

R₂ is hydrogen or an optionally substituted substituent;

R₃ is hydrogen or an optionally substituted substituent;

R₄ is hydrogen or an optionally substituted substituent;

R₅ is hydrogen or an optionally substituted substituent;

R₆ is hydrogen or an optionally substituted substituent;

R₇ is hydrogen or an optionally substituted substituent; and

R₈ is hydrogen or an optionally substituted substituent;

wherein optionally any two or more of R₁, R₂, R₃, R₄, R₅, R₆, R₇, or R₈ may be joined together to form one or more rings.

Further provided herein is a method of treating an inflammatory or autoimmune disorders, such as for example, multiple sclerosis using T cells stimulated by dendritic cells with autoimmune antigens, such as for example, myelin associated proteins, and optionally along with a GITR/GITRL antagonist or T-eff modified with a GITR/GITRL antagonist ex vivo, wherein the antagonist is compound of Formula II, and in particular RMGL 171104.

The compound of Formula (II):

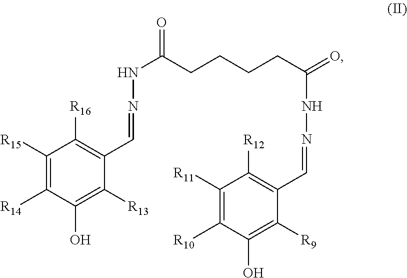

or a pharmaceutically acceptable salt, ester or prodrug thereof;
wherein:

R₉ is hydrogen or an optionally substituted substituent;

R₁₀ is hydrogen or an optionally substituted substituent;

R₁₁ is hydrogen or an optionally substituted substituent;

R₁₂ is hydrogen or an optionally substituted substituent;

R₁₃ is hydrogen or an optionally substituted substituent;

R₁₄ is hydrogen or an optionally substituted substituent;

R₁₅ is hydrogen or an optionally substituted substituent; and

R₁₆ is hydrogen or an optionally substituted substituent;

wherein optionally any two or more of R₉, R₁₀, R₁₁, R₁₂, R₁₃, R₁₄, R₁₅, or R₁₆ may be joined together to form one or more rings.

In particular,

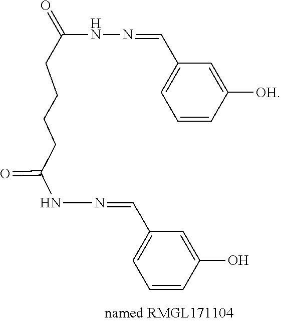 [SMILES: O=C(CCCCC(=O)NN=Cc1cccc(O)c1)NN=Cc1cccc(O)c1]

Provided herein are GITR antagonists selected from any one or more of the compounds having the structure described in Formula II.

In another aspect, the invention is directed to a method for treating an inflammatory disease in a subject in need thereof comprising administering to the subject a therapeutically effective amount of a GITR/GITRL antagonist described herein together with T-cells that have been activated with antigen presenting cells such as dendritic cells with autoimmune antigens.

The GITR/GITRL antagonist may be co-administered with existing therapies for inflammatory disease to the subject or sequentially administered. The autoimmune disease may be rheumatoid arthritis, osteoarthritis, asthma, dermatitis, psoriasis, cystic fibrosis, post transplantation late and chronic solid organ rejection, multiple sclerosis, systemic lupus erythematosus, Sjogren's syndrome, Hashimoto thyroiditis, polymyositis, scleroderma, Addison disease, vitiligo, pernicious anemia, glomerulonephritis and pulmonary fibrosis, inflammatory bowel diseases, autoimmune diabetes, diabetic retinopathy, rhinitis, ischemia-reperfusion injury, post-angioplasty restenosis, chronic obstructive pulmonary diseases (COPD), Grave's disease, gastrointestinal allergies, conjunctivitis, atherosclerosis, coronary artery disease, angina, cancer metastasis, small artery disease, graft-versus-host disease, or mitochondrial related syndrome. Preferably, the autoimmune disease may be inflammatory bowel disease.

In another aspect, the invention is directed to a method for treating inflammatory disease in a subject in need thereof comprising administering to the subject a therapeutically effective amount of T cells activated by antigen presenting cells carrying inflammatory factor antigen or autoimmune antigen ex vivo. GITR/GITRL antagonist described herein by either in vivo or by administering engineered T cells that have been enriched or expanded for T-reg in vivo or ex vivo, wherein the T-reg cells are enriched or expanded and T-eff cells are modified by contacting the T-eff cells with a GITR/GITRL antagonist with or without the presence of T-reg cells. The T-eff or T-reg cells may be autologous or allogeneic relative to the subject. Such obtained cells may be administered to the patient.

In another aspect, the invention is directed to a method of enriching or expanding a population of activated T cells, including T-reg cells comprising contacting T cells with a GITR/GITRL antagonist with or without the presence of T-eff cells. Preferably, the T-reg cells are initially present. And the T-eff and T-reg cells may be present in a starting ratio of about 1:1.

Further provided herein are compositions comprising the GITR antagonists as described herein. Also provided are methods for using the GITR antagonists for treating inflammatory diseases, in particular, autoimmune diseases in a subject by administering to the subject a therapeutically effective amount of the compositions comprising the GITR antagonists. In some embodiments, the methods further comprise administering existing therapies for autoimmune diseases to the subject.

### BRIEF DESCRIPTION OF THE DRAWINGS

Exemplary embodiments are illustrated in referenced figures. It is intended that the embodiments and figures disclosed herein are to be considered illustrative rather than restrictive.

FIGS. 1A-1B show the results of where PBMC was isolated (Ficoll, GE Healthcare) from the WBC cone collected from healthy platelet donor. Cells were washed and passed through 40 um cell strainer before being stained with T cell surface antibodies. Then cells were put on cell sorter (BD FACSARIA III). Specific cell populations were collected as follows: CD4⁺CD25⁻ cells (T effector cells), CD4⁺CD25⁺CD45RA⁺CD127⁻ cells (T regulatory cells) and CD3⁻ cells (serve as Antigen Presenting Cells, APC). T effector cells were labeled with CellTrace CFSE (Invitrogen), heavily washed before cell number counting. Effector cells and T-regs were then mixed together at 1:1 ratio in culture media (RPMI 1640, 10% FBS, Pen-Strep and 1% NEAA) which enhanced with anti-CD3 (3 ug/ml) anti-CD28 (2 ug/ml) antibodies. APCs were treated with Mitomycin (50 ug/ml) for 30 minutes at 37° C., 5% CO₂ incubator, then added to culture mix (APC:T-eff 2:1) as a proliferation co-stimulator. Cell mixture was incubated at 37° C., 5% CO₂ for 6 days before being re-stained with T cell surface markers (CD4, CD25) and sent for FACS analysis. (A) T-eff fully stimulated; (B) T-eff fully stimulated+T-reg (1:1).

FIGS. 2A-2C show FACS analysis for (A) T-eff fully stimulated+11702 (5 ul); (B) T-eff fully stimulated+11702 (25 ul); (C) T-eff fully stimulated+11702 (50 ul).

FIGS. 2D-2F show FACS analysis for (A) T-eff fully stimulated+11702 (5 ul)+T-reg; (B) T-eff fully stimulated+11702 (25 ul)+T-reg; (C) T-eff fully stimulated+11702 (50 ul)+T-reg.

FIGS. 3A-3C show FACS analysis for (A) T-eff fully stimulated+11704 (5 ul); (B) T-eff fully stimulated+11704 (25 ul); (C) T-eff fully stimulated+11704 (50 ul).

FIGS. 3D-3F show FACS analysis for (A) T-eff fully stimulated+11704 (5 ul)+T-reg; (B) T-eff fully stimulated+11704 (25 ul)+T-reg; (C) T-eff fully stimulated+11704 (50 ul)+T-reg.

FIG. 4 shows summary table of the effects of the agonist and antagonist compounds and the effect on T Cell effector proliferation change. PBMC was isolated and specific human T cell populations were collected as follows: CD4⁺CD25⁻ cells (T effector cells), CD4⁺CD25⁺CD45RA⁺CD127⁻ cells (T regulatory cells) and CD3⁻ cells (serve as Antigen Presenting Cells, APC). T effector cells were labeled with CellTrace CFSE and effector cells and T-regs were then mixed together at 1:1 ratio in culture media (RPMI 1640, 10% FBS, Pen-Strep and 1% NEAA) which enhanced with anti-CD3 (3 ug/ml) anti-CD28 (2 ug/ml) antibodies. APCs were treated with Mitomycin (50 ug/ml) for 30 minutes at 37° C., 5% CO₂ incubator, then added to culture mix (APC:T-eff 2:1) as a proliferation co-stimulator, incubated, and sent for FACS analysis. Molecule 11702 agonist and 11704 antagonist were added to treat groups respectively at a concentration gradient of 5 uM, 25 uM and 50 uM.

FIG. 5 shows summary graph setting forth the Table of FIG. 4.

FIGS. 6A-6C show that GITR agonist 11702 inhibits melanoma growth through T-eff proliferation and T-reg inhibition in the tumor. After implantation of B16 melanoma, C57 BL mice underwent treatment with 11702 GITR agonist or DMSO control. (A) Animals lived longer after GITR agonist intraperitoneal 30 mg/kg treatment twice per week (p=0.0333, log rank). (B) Tumor volume was inhibited in 11702 treated animals (p<0.05, Anova). (C) FACs analysis of tumor infiltrating lymphocytes demonstrated the increased presence of activated CD4+ cells and increased effector memory cytotoxic CD8+ T cells. Both of these groups showed increased PD-1 expression suggesting increased IFN gamma induced upregulation of PD-1 and invoking the potential synergy of this agent with PD-1 checkpoint blockade.

FIGS. 7-16 show that T cells can be significantly activated by autologous Dendritic Cells pulsed with CSC 6 lysate or acid-eluted peptides after 8-13 days' culturing. CSC 6 lysate gives a greater degree of response vs. acid eluted peptides.

FIGS. 17-19 show that the inventive method results in minimal autoimmunity.

FIG. 20 shows that T cell activation surface markers show upregulation of CD137, CD154, CD69, CD45RO and HLA-DR after 12-day culture and activation. Meanwhile, CD4+ population increases and CD8+ population decreases following T cell expansion.

FIG. 21 shows that when targeting T2 cells, T cells activated by epitope-loaded DCs (TP12) show significant immune responses compared to those T cells activated by no-epitope DCs (TNP12). The antigen specific immune response occurs only when T2 target cells are loaded with antigen.

FIG. 22 shows that both types of activated T cells (TP and TNP) respond well to all four CSC line cells compared to naïve T cells (T0). But T cells activated by epitope-loaded DCs (TP) secrete more than 1.5 fold of Interferon-gamma towards target cells than those T cells activated by no-epitope DCs (TNP), due to the recognition of cancer stem cell epitopes and consequent reaction.

FIG. 23 shows that T cell surface marker staining shows activation signs as early as day 5, particularly CD137 and CD69.

FIG. 24 shows that T cells activated by epitope-pulsed DCs (TP12) secrete 2-fold more IFN-γ when encountering epitope-loaded T2 cells in contrast to unloaded T2 cells. T cells activated by no-epitope DCs (TNP12) show no significant difference when targeting two types of T2 cells (with or without epitope-loading).

FIG. 25 shows that T cells activated by epitope-pulsed DCs (TP12) respond stronger to established GBM cancer stem cell lines (all HLA-A2+) compared to naïve T cells (T0) and T cells activated by no-epitope DCs (TNP12), suggesting killing efficacy against cancer stem cells.

FIG. 26 shows that 19-day activation/expansion of T cells also show up-regulated T cell surface activation markers CD137, CD69, HLA-DR, CD45RO and CD154.

FIG. 27 shows that T cells activated by epitope-loaded DCs and expanded for 19 days (TP19) show much stronger antigen-specific response to T2 cells loaded with the same epitopes than unloaded group, compared to those T cells activated by no-epitope DCs (TNP19).

FIG. 28 shows that 19-day T cell Elispot data show mixed Interferon-gamma secretion results. All activated T cells (TP19 and TNP19) have stronger immune responses towards CSC lines compared to naïve T cells (T0). However, such increased responses have no significant difference between TP19 and TNP19 cells. These results suggested that the prolonged 19 day culture promoted loss of antigen specific killing of the T cells.

FIG. 29 shows surface marker staining. After 13 days of culture, CD4+ increase, CD8+ decrease. All other activation markers are upregulated.

FIG. 30 shows that T cells activated by epitope-pulsed DCs (TP13) secrete more IFN-γ when encountered with peptide-loaded T2 cells compared to unloaded T2 cells. T cells activated by empty DCs (TNP13) show no such differences which demonstrates antigen-specific cytotoxicity.

FIG. 31 shows Elispot assay—T cell response to CSC lines. TP13 showing significantly higher interferon-gamma secretion than TNP13 and naïve T0 cells.

FIG. 32 shows in vitro assay for autoimmune effect by T cells. PHA-blasts (HT1) were co-cultured with T0, TNP12 and TP12 at E:T ratio of 5:1. Percentage of dead PHA-blasts was indicated by % parent CFSE+/eFluor780+ population. % parent of PHA-blast without any T cells (BL only) was used to calculate % specific lysis for T0, TNP12 and TP12, showing all less than 10% of dead PHA-blasts.

FIG. 33 shows in vitro assay for autoimmune effect by T cells. PHA-blasts (HT1) were co-cultured with T0, TNP19 and TP19 at E:T ratio of 5:1. Percentage of dead PHA-blasts was indicated by % parent CFSE+/eFluor780+ population. % parent of PHA-blast without any T cells (BL only) was used to calculate % specific lysis for T0, TNP19 and TP19, showing all less than 10% of dead PHA-blasts.

FIG. 34 shows in vitro assay for autoimmune effect by T cells. PHA-blasts (HT2) were co-cultured with TP19 at E:T ratio of 1:10. Percentage of dead PHA-blasts was indicated by % parent CTV+/eFluor780+ population. % parent of PHA-blast without any T cells (BL only) was used to calculate % specific lysis for TP19, showing all less than 5% of dead PHA-blasts.

FIG. 35 shows in vitro assay for autoimmune effect by T cells. PHA-blasts (HT3) were co-cultured with T0, TNP13 and TP13 at E:T ratio of 20:1. Percentage of dead PHA-blasts was indicated by % parent CTV+/eFluor780+ population. % parent of PHA-blast without any T cells (BL only) was used to calculate % specific lysis for T0, TNP13 and TP13, showing all less than 5% of dead PHA-blasts.

FIG. 36 shows activated T Cell manufacturing flow chart.

### DETAILED DESCRIPTION

All references cited herein are incorporated by reference in their entirety as though fully set forth. Unless defined otherwise, technical and scientific terms used herein have the same meaning as commonly understood by one of ordinary skill in the art to which this invention belongs. Definitions of common terms in molecular biology may be found in Benjamin Lewin, Genes V, published by Oxford University Press, 1994 (ISBN 0-19-854287-9); Kendrew et al. (eds.), The Encyclopedia of Molecular Biology, published by Blackwell Science Ltd., 1994 (ISBN 0-632-02182-9); and Robert A. Meyers (ed.), Molecular Biology and Biotechnology: a Comprehensive Desk Reference, published by VCH Publishers, Inc., 1995 (ISBN 1-56081-569-8). Allen et al., Remington: The Science and Practice of Pharmacy 22^{nd} ed., Pharmaceutical Press (Sep. 15, 2012); Hornyak et al., Introduction to Nanoscience and Nanotechnology, CRC Press (2008); Singleton and Sainsbury, Dictionary of Microbiology and Molecular Biology 3^{rd} ed., revised ed., J. Wiley & Sons (New York, N.Y. 2006); Smith, March's Advanced Organic Chemistry Reactions, Mechanisms and Structure 7^{th} ed., J. Wiley & Sons (New York, N.Y. 2013); Singleton, Dictionary of DNA and Genome Technology 3^{rd} ed., Wiley-Blackwell (Nov. 28, 2012); and Green and Sambrook, Molecular Cloning: A Laboratory Manual 4th ed., Cold Spring Harbor Laboratory Press (Cold Spring Harbor, N.Y. 2012), provide one skilled in the art with a general guide to many of the terms used in the present application. For references on how to prepare antibodies, see Greenfield, Antibodies A Laboratory Manual 2^{nd} ed., Cold Spring Harbor Press (Cold Spring Harbor N.Y., 2013); Kohler and Milstein, Derivation of specific antibody-producing tissue culture and tumor lines by cell fusion, Eur. J. Immunol. 1976 July, 6(7):511-9; Queen and Selick, Humanized immunoglobulins, U.S. Pat. No. 5,585,089 (1996 December); and Riechmann et al., Reshaping human antibodies for therapy, Nature 1988 Mar. 24, 332(6162):323-7.

One skilled in the art will recognize many methods and materials similar or equivalent to those described herein, which could be used in the practice of the present invention. Other features and advantages of the invention will become apparent from the following detailed description, taken in conjunction with the accompanying drawings, which illustrate, by way of example, various features of embodiments of the invention. Indeed, the present invention is in no way limited to the methods and materials described. For convenience, certain terms employed herein, in the specification, examples and appended claims are collected here.

Unless stated otherwise, or implicit from context, the following terms and phrases include the meanings provided below. Unless explicitly stated otherwise, or apparent from context, the terms and phrases below do not exclude the meaning that the term or phrase has acquired in the art to which it pertains. Unless otherwise defined, all technical and scientific terms used herein have the same meaning as commonly understood by one of ordinary skill in the art to which this invention belongs. It should be understood that this invention is not limited to the particular methodology, protocols, and reagents, etc., described herein and as such can vary. The definitions and terminology used herein are provided to aid in describing particular embodiments, and are not intended to limit the claimed invention, because the scope of the invention is limited only by the claims.

As used herein, “activated T cell” refers to autologous T cells from cancer patients that are activated in vitro by certain cytokines in a certain sequence and primed with certain cytotoxic antigens and helper antigens to generate T cells that recognize cancer stem cells. T cells are primed with dendritic cells loaded with certain tumor-specific epitopes, from both cytotoxic and/or helper antigens. This method of activation and priming generates potent antigen specific T cells that can recognize and kill cancer stem cells. Administering these activated T cells into patients suffering from cancer would be expected to kill the cancer stem cells that propagate the patient's tumor, thereby achieving a therapeutic response.

As used herein, “RMGL171102”, “RMGL171103” and “RMGL171104” are interchangeably referred to as compound 11702, 11703 and 11704, respectively.

As used herein, “cell therapy” is also considered as ex vivo therapy, in that cells are grown or treated outside of the body and are then returned to the patient by injection or transplantation. The treated cells may be autologous or allogeneic relative to the patient.

As used herein the term “comprising” or “comprises” is used in reference to compositions, methods, and respective component(s) thereof, that are useful to an embodiment, yet open to the inclusion of unspecified elements, whether useful or not. It will be understood by those within the art that, in general, terms used herein are generally intended as “open” terms (e.g., the term “including” should be interpreted as “including but not limited to,” the term “having” should be interpreted as “having at least,” the term “includes” should be interpreted as “includes but is not limited to,” etc.). Although the open-ended term “comprising,” as a synonym of terms such as including, containing, or having, is used herein to describe and claim the invention, the present invention, or embodiments thereof, may alternatively be described using alternative terms such as “consisting of” or “consisting essentially of.”

Unless stated otherwise, the terms “a” and “an” and “the” and similar references used in the context of describing a particular embodiment of the application (especially in the context of claims) can be construed to cover both the singular and the plural. The recitation of ranges of values herein is merely intended to serve as a shorthand method of referring individually to each separate value falling within the range. Unless otherwise indicated herein, each individual value is incorporated into the specification as if it were individually recited herein. All methods described herein can be performed in any suitable order unless otherwise indicated herein or otherwise clearly contradicted by context. The use of any and all examples, or exemplary language (for example, “such as”) provided with respect to certain embodiments herein is intended merely to better illuminate the application and does not pose a limitation on the scope of the application otherwise claimed. The abbreviation, “e.g.” is derived from the Latin exempli gratia, and is used herein to indicate a non-limiting example. Thus, the abbreviation “e.g.” is synonymous with the term “for example.” No language in the specification should be construed as indicating any non-claimed element essential to the practice of the application.

“Optional” or “optionally” means that the subsequently described circumstance may or may not occur, so that the description includes instances where the circumstance occurs and instances where it does not.

As used herein the term “agent” or “agents” means any one or more of a protein, peptide, peptidomimetic, compound, chemical compound, small molecule, organic compound, inorganic compound, antisense compound, antibody, protease inhibitor, hormone, chemokine, cytokine, or compound of the invention as described herein, or other molecule of interest. In one embodiment, the agent is a GITR agonist (for example, peptides having the sequence set forth in SEQ ID NO: 1 or SEQ ID NO: 2, or agents having the structure of Formula I, in particular compound named RMGL171102 (aka compound 11702). In a further embodiment, the agent is a GITR antagonist (for example, agents having the structure of Formula II). In a further embodiment, the agent is a GITR antagonist named RMGL171104 (aka compound 11704).

As used herein, the terms “treat,” “treatment,” “treating,” or “amelioration” when used in reference to a disease, disorder or medical condition, refer to both therapeutic treatment and prophylactic or preventative measures, wherein the object is to prevent, reverse, alleviate, ameliorate, inhibit, lessen, slow down or stop the progression or severity of a symptom or condition. The term “treating” includes reducing or alleviating at least one adverse effect or symptom of a condition. Treatment is generally “effective” if one or more symptoms or clinical markers are reduced. Alternatively, treatment is “effective” if the progression of a disease, disorder or medical condition is reduced or halted. That is, “treatment” includes not just the improvement of symptoms or markers, but also a cessation or at least slowing of progress or worsening of symptoms that would be expected in the absence of treatment. Also, “treatment” may mean to pursue or obtain beneficial results, or lower the chances of the individual developing the condition even if the treatment is ultimately unsuccessful. Those in need of treatment include those already with the condition as well as those prone to have the condition or those in whom the condition is to be prevented.

As used herein, a therapeutic that “prevents” a disorder or condition refers to a compound that, in a statistical sample, reduces the occurrence of the disorder or condition in the treated sample relative to an untreated control sample, or delays the onset or reduces the severity of one or more symptoms of the disorder or condition relative to the untreated control sample.

The terms “decrease”, “reduced”, “reduction”, or “inhibit” are all used herein to mean a decrease or lessening of a property, level, or other parameter by a statistically significant amount. In some embodiments, “reduce,” “reduction” or “decrease” or “inhibit” typically means a decrease by at least 10% as compared to a reference level (e.g., the absence of a given treatment) and can include, for example, a decrease by at least about 10%, at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, at least about 95%, at least about 98%, at least about 99%, or more. As used herein, “reduction” or “inhibition” does not encompass a complete inhibition or reduction as compared to a reference level. “Complete inhibition” is a 100% inhibition as compared to a reference level. A decrease can be preferably down to a level accepted as within the range of normal for an individual without a given disorder.

The terms “increased”, “increase” or “enhance” or “activate” are all used herein to generally mean an increase of a property, level, or other parameter by a statically significant amount; for the avoidance of any doubt, the terms “increased”, “increase” or “enhance” or “activate” means an increase of at least 10% as compared to a reference level, for example an increase of at least about 20%, or at least about 30%, or at least about 40%, or at least about 50%, or at least about 60%, or at least about 70%, or at least about 80%, or at least about 90% or up to and including a 100% increase or any increase between 10-100% as compared to a reference level, or at least about a 2-fold, or at least about a 3-fold, or at least about a 4-fold, or at least about a 5-fold or at least about a 10-fold increase, at least about a 20-fold increase, at least about a 50-fold increase, at least about a 100-fold increase, at least about a 1000-fold increase or more as compared to a reference level.

A “cancer” or “tumor” as used herein refers to an uncontrolled growth of cells which interferes with the normal functioning of the bodily organs and systems. A subject that has a cancer or a tumor is a subject having objectively measurable cancer cells present in the subject's body. Included in this definition are benign and malignant cancers, as well as dormant tumors or micrometastatses. Cancers which migrate from their original location and seed vital organs can eventually lead to the death of the subject through the functional deterioration of the affected organs. Examples of cancer include, but are not limited to B-cell lymphomas (Hodgkin's lymphomas and/or non-Hodgkins lymphomas), brain tumor, breast cancer, colon cancer, lung cancer, hepatocellular cancer, gastric cancer, pancreatic cancer, cervical cancer, ovarian cancer, liver cancer, bladder cancer, cancer of the urinary tract, thyroid cancer, renal cancer, carcinoma, melanoma, head and neck cancer, brain cancer such as glioblastoma, and prostate cancer, including but not limited to androgen-dependent prostate cancer and androgen-independent prostate cancer.

The term “effective amount” or “therapeutically effective amount” as used herein refers to the amount of one or more GITR agonists or GITR antagonists, or amount of pharmaceutical compositions comprising one or more GITR agonists or GITR antagonists as disclosed herein, to decrease at least one or more symptom of the disease or disorder, and relates to a sufficient amount of the pharmacological composition to provide the desired effect. The phrase “therapeutically effective amount” as used herein means a sufficient amount of the composition to treat a disorder, at a reasonable benefit/risk ratio applicable to any medical treatment.

“Peptidomimetic” as used herein is a small protein-like chain designed to mimic a protein function. They may be modifications of an existing peptide or newly designed to mimic known peptides. They may be, for example peptoids and/or β-peptides and/or D-peptides.

“Recombinant virus” refers to a virus that has been genetically altered (e.g., by the addition or insertion) of a heterologous nucleic acid construct into the particle.

A “gene” or “coding sequence” or a sequence which “encodes” a particular protein or peptide is a nucleic acid molecule that is transcribed (in the case of DNA) and translated (in the case of mRNA) into a polypeptide in vitro or in vivo when placed under the control of appropriate regulatory sequences. The boundaries of the gene are determined by a start codon at the 5′ (i.e., amino) terminus and a translation stop codon at the 3′ (i.e., carboxy) terminus. A gene can include, but is not limited to, cDNA from prokaryotic or eukaryotic mRNA, genomic DNA sequences from prokaryotic or eukaryotic DNA, and even synthetic DNA sequences. A transcription termination sequence will usually be located 3′ to the gene sequence.

The term “control elements” refers collectively to promoter regions, polyadenylation signals, transcription termination sequences, upstream regulatory domains, origins of replication, internal ribosome entry sites (“IRES”), enhancers, and the like, which collectively provide for the replication, transcription and translation of a coding sequence in a recipient cell. Not all of these control elements need always be present, so long as the selected coding sequence is capable of being replicated, transcribed and translated in an appropriate host cell.

The term “promoter region” is used herein in its ordinary sense to refer to a nucleotide region including a DNA regulatory sequence, wherein the regulatory sequence is derived from a gene which is capable of binding RNA polymerase and initiating transcription of a downstream (3′-direction) coding sequence.

“Operably linked” refers to an arrangement of elements wherein the components so described are configured so as to perform their usual function. Thus, control elements operably linked to a coding sequence are capable of effecting the expression of the coding sequence. The control elements need not be contiguous with the coding sequence, so long as they function to direct the expression thereof. Thus, for example, intervening untranslated yet transcribed sequences can be present between a promoter sequence and the coding sequence and the promoter sequence can still be considered “operably linked” to the coding sequence.

“Gene transfer” or “gene delivery” refers to methods or systems for reliably inserting foreign DNA into host cells. Such methods can result in transient expression of non-integrated transferred DNA, extrachromosomal replication and expression of transferred replicons (e.g., episomes), or integration of transferred genetic material into the genomic DNA of host cells. Gene transfer provides a unique approach for the treatment of acquired and inherited diseases. A number of systems have been developed for gene transfer into mammalian cells. See, e.g., U.S. Pat. No. 5,399,346. Examples of well-known vehicles for gene transfer include adenovirus and recombinant adenovirus (RAd), adeno-associated virus (AAV), herpes simplex virus type 1 (HSV-1), and lentivirus (LV).

“Genetically modified cells”, “genetically engineered cells”, or “modified cells” as used herein refer to cells that express the polynucleotide encoding polypeptides having the sequence of any one or more of SEQ ID NO: 1 or SEQ ID NO: 2 or a variant, derivative, pharmaceutical equivalent, peptidomimetic or an analog thereof.

“Naked DNA” as used herein refers to DNA encoding a polypeptide having the sequence of any one or more of SEQ ID NO: 1 or SEQ ID NO: 2 or a variant, derivative, pharmaceutical equivalent, peptidomimetic or an analog thereof, cloned in a suitable expression vector in proper orientation for expression. Viral vectors which may be used include but are not limited SIN lentiviral vectors, retroviral vectors, foamy virus vectors, adeno-associated virus (AAV) vectors, hybrid vectors and/or plasmid transposons (for example sleeping beauty transposon system) or integrase-based vector systems. Other vectors that may be used in connection with alternate embodiments of the invention will be apparent to those of skill in the art.

“Polynucleotide” as used herein includes but is not limited to DNA, RNA, cDNA (complementary DNA), mRNA (messenger RNA), rRNA (ribosomal RNA), shRNA (small hairpin RNA), snRNA (small nuclear RNA), snoRNA (short nucleolar RNA), miRNA (microRNA), genomic DNA, synthetic DNA, synthetic RNA, and/or tRNA.

The term “transfection” is used herein to refer to the uptake of foreign DNA by a cell. A cell has been “transfected” when exogenous DNA has been introduced inside the cell membrane. A number of transfection techniques are generally known in the art. See, e.g., Graham et al. Virology, 52:456 (1973); Sambrook et al. Molecular Cloning, a laboratory manual, Cold Spring Harbor Laboratories, New York (1989); Davis et al., Basic Methods in Molecular Biology, Elsevier (1986), and Chu et al. Gene 13:197 (1981). Such techniques can be used to introduce one or more exogenous DNA moieties, such as a plasmid vector and other nucleic acid molecules, into suitable host cells. The term refers to both stable and transient uptake of the genetic material.

“Vector”, “cloning vector” and “expression vector” as used herein refer to the vehicle by which a polynucleotide sequence (e.g. a foreign gene) can be introduced into a host cell, so as to transform the host and promote expression (e.g. transcription and translation) of the introduced sequence. Vectors include plasmids, phages, viruses, etc.

“Beneficial results” or “desired results” may include, but are in no way limited to, lessening or alleviating the severity of the disease condition, preventing the disease condition from worsening, curing the disease condition, preventing the disease condition from developing, lowering the chances of a patient developing the disease condition, decreasing morbidity and mortality, and prolonging a patient's life or life expectancy. As non-limiting examples, “beneficial results” or “desired results” may be alleviation of one or more symptom(s), diminishment of extent of the deficit, stabilized (i.e., not worsening) state of cancer, delay or slowing of cancer, and amelioration or palliation of symptoms associated with cancer.

“Diseases”, “conditions” and “disease conditions,” as used herein may include, but are in no way limited to any form of cancer or autoimmune diseases.

As used herein, the term “administering,” refers to the placement of an agent or a composition as disclosed herein into a subject by a method or route which results in at least partial localization of the agents or composition at a desired site. “Route of administration” may refer to any administration pathway known in the art, including but not limited to oral, topical, aerosol, nasal, via inhalation, anal, intra-anal, peri-anal, transmucosal, transdermal, parenteral, enteral, or local. “Parenteral” refers to a route of administration that is generally associated with injection, including intratumoral, intracranial, intraventricular, intrathecal, epidural, intradural, intraorbital, infusion, intracapsular, intracardiac, intradermal, intramuscular, intraperitoneal, intrapulmonary, intraspinal, intrasternal, intrathecal, intravascular, intravenous, intraarterial, subarachnoid, subcapsular, subcutaneous, transmucosal, or transtracheal. Via the parenteral route, the agent or composition may be in the form of solutions or suspensions for infusion or for injection, or as lyophilized powders. Via the enteral route, the agent or composition can be in the form of capsules, gel capsules, tablets, sugar-coated tablets, syrups, suspensions, solutions, powders, granules, emulsions, microspheres or nanospheres or lipid vesicles or polymer vesicles allowing controlled release. Via the topical route, the agent or composition can be in the form of aerosol, lotion, cream, gel, ointment, suspensions, solutions or emulsions. In an embodiment, agent or composition may be provided in a powder form and mixed with a liquid, such as water, to form a beverage. In accordance with the present invention, “administering” can be self-administering. For example, it is considered as “administering” that a subject consumes a composition as disclosed herein.

As used herein, a “subject” means a human or animal. Usually the animal is a vertebrate such as a primate, rodent, domestic animal or game animal. Primates include chimpanzees, cynomologous monkeys, spider monkeys, and macaques, e.g., Rhesus. Rodents include mice, rats, woodchucks, ferrets, rabbits and hamsters. Domestic and game animals include cows, horses, pigs, deer, bison, buffalo, feline species, e.g., domestic cat, and canine species, e.g., dog, fox, wolf. The terms, “patient”, “individual” and “subject” are used interchangeably herein. In an embodiment, the subject is mammal. The mammal can be a human, non-human primate, mouse, rat, dog, cat, horse, or cow, but are not limited to these examples. In addition, the methods described herein can be used to treat domesticated animals and/or pets. In one embodiment, the subject is a human.

“Mammal” as used herein refers to any member of the class Mammalia, including, without limitation, humans and nonhuman primates such as chimpanzees and other apes and monkey species; farm animals such as cattle, sheep, pigs, goats and horses; domestic mammals such as dogs and cats; laboratory animals including rodents such as mice, rats and guinea pigs, and the like. The term does not denote a particular age. Thus, adult and newborn subjects, as well as fetuses, are intended to be included within the scope of this term.

A subject can be one who has been previously diagnosed with or identified as suffering from or having a condition in need of treatment (e.g., cancer or autoimmune diseases) or one or more complications related to the condition, and optionally, have already undergone treatment for the condition or the one or more complications related to the condition. Alternatively, a subject can also be one who has not been previously diagnosed as having a condition or one or more complications related to the condition. For example, a subject can be one who exhibits one or more risk factors for a condition or one or more complications related to the condition or a subject who does not exhibit risk factors. For example, a subject can be one who exhibits one or more symptoms for a condition or one or more complications related to the condition or a subject who does not exhibit symptoms. A “subject in need” of diagnosis or treatment for a particular condition can be a subject suspected of having that condition, diagnosed as having that condition, already treated or being treated for that condition, not treated for that condition, or at risk of developing that condition.

By “at risk of” is intended to mean at increased risk of, compared to a normal subject, or compared to a control group, e.g. a patient population. Thus a subject carrying a particular marker may have an increased risk for a specific disease or disorder, and be identified as needing further testing. “Increased risk” or “elevated risk” mean any statistically significant increase in the probability, e.g., that the subject has the disorder. The risk is preferably increased by at least 10%, more preferably at least 20%, and even more preferably at least 50% over the control group with which the comparison is being made.

Immunosuppressive Drug includes any agent or compound having the ability to decrease the body's immune system responses. In some embodiments, the immunosuppressive drug is a corticosteroid. In other embodiments, the immunosuppressive drug is a small molecule (such as cyclosporine) or a monoclonal antibody (such as a cytokine blocker).

Non-Steroidal Anti-Inflammatory Drug (NSAID): A type of anti-inflammatory agent that works by inhibiting the production of prostaglandins. NSAIDS exert anti-inflammatory, analgesic and antipyretic actions. Examples of NSAIDS include ibuprofen, ketoprofen, piroxicam, naproxen, sulindac, aspirin, choline subsalicylate, diflunisal, fenoprofen, indomethacin, meclofenamate, salsalate, tolmetin and magnesium salicylate.

The term “statistically significant” or “significantly” refers to statistical significance and generally means at least two standard deviation (2SD) away from a reference level. The term refers to statistical evidence that there is a difference. It is defined as the probability of making a decision to reject the null hypothesis when the null hypothesis is actually true.

As used herein, the term “co-administer” refers to administration of two or more therapies or two or more therapeutic agents (e.g., GITR agonist and additional anti-cancer therapies; or GITR antagonists and anti-autoimmune diseases therapies) within a 24 hour period of each other, for example, as part of a clinical treatment regimen. In other embodiments, “co-administer” refers to administration within 12 hours, within 6 hours, within 5 hours, within 4 hours, within 3 hours, within 2 hours, within 1 hour, within 45, within 30 minutes, within 20, within 15 minutes, within 10 minutes, or within 5 minutes of each other. In other embodiments, “co-administer” refers to administration at the same time, either as part of a single formulation or as multiple formulations that are administered by the same or different routes. For example, when the GITR agonist and the additional anti-cancer therapy are administered in different pharmaceutical compositions or at different times, routes of administration can be same or different. For example, when the GITR antagonist and the additional anti-autoimmune disease therapy are administered in different pharmaceutical compositions or at different times, routes of administration can be same or different.

Dendritic Cell-Based Immunotherapy

The present invention is directed to T cell therapy for cancer, and in particular, T-cell/B-cell lymphomas (Hodgkin's lymphomas and/or non-Hodgkins lymphomas), brain tumor, breast cancer, colon cancer, lung cancer, hepatocellular cancer, gastric cancer, pancreatic cancer, cervical cancer, ovarian cancer, liver cancer, bladder cancer, cancer of the urinary tract, thyroid cancer, renal cancer, carcinoma, skin cancer, head and neck cancer, brain cancer, and prostate cancer, androgen-dependent prostate cancer and androgen-independent prostate cancer, and further in particular glioblastoma.

While much of exemplification provided herein is for treating glioblastoma, the principles disclosed in the present application are contemplated to be not limited to glioblastoma, but are applicable for all types of cancers as discussed above.

In particular detail, Applicant describes activating autologous T cells with dendritic cells loaded with either: proteins isolated from the patient's cancer stem cells grown in culture from their own tumor or from MHC-1 associated peptides isolated from the patient's own cancer stem cells grown from their tumor, or from CTL (Cytotoxic T Lymphocyte) peptides to known antigens highly expressed on cancer stem cells. The list of CTL antigens and helper antigens are listed below.

| | | |
|---|---|---|
| gp100210M (209-217) | HLA-A2 | H-IMDQVPFSV-OH |
| | | (SEQ ID NO: 6) |
| MAGE1 (161-169) | HLA-A1 | H-EADPTGHSY-OH |
| | | (SEQ ID NO: 7) |
| NY-ESO-1 (157-165) | HLA-A2 | H-SLLMWITQC-OH |
| | | (SEQ ID NO: 8) |
| TRP-2 (180-188) | HLA-A2 | H-SVYDFFVWL-OH |
| | | (SEQ ID NO: 9) |
| EphA2 (883-891) | HLA-A2 | H-TLADFDPRV-OH |
| | | (SEQ ID NO: 10) |
| AIM2 (14-23) | HLA-A1 | H-RSDSGQQARY-OH |
| | | (SEQ ID NO: 11) |
| HER2/neu (773-782) | HLA-A2 | H-VMAGVGSPYV-OH |
| | | (SEQ ID NO: 12) |
| IL-13Ra2 (345-353) | HLA-A2 | H-WLPFGFILI-OH |
| | | (SEQ ID NO: 13) |
| MAGE-A1 (278-286) | HLA-A2 | H-KVLEYVIKV-OH |
| | | (SEQ ID NO: 14) |

The following peptides are used as helper peptides to the previous CTL peptides: gp100 (576-590) SLAVVSTQLIMPGQE (SEQ ID NO:15); NY-ESO-1 (119-143) PGVLLKEFTVSGNILTIRLTAADHR (SEQ ID NO:16); TRP-2 (60-74) QCTEVRADTRPWSGP (SEQ ID NO:17); TRP-2(149-163) KKRVHPDYVITTQHWL (SEQ ID NO:18); EphA2 (663-677) EAGIMGQFSHHNIIR (SEQ ID NO:19); HER2/neu (883-899) KVPIKWMALESILRRRF (SEQ ID NO:20); HER2/neu (369-384) KIFGSLAFLPESFDGDPA (SEQ ID NO:21); HER2/neu (688-703) RRLLQETELVEPLTPS (SEQ ID NO:22); and HER2/neu (671-684) ELVSEFSRMARDPQ (SEQ ID NO:23).

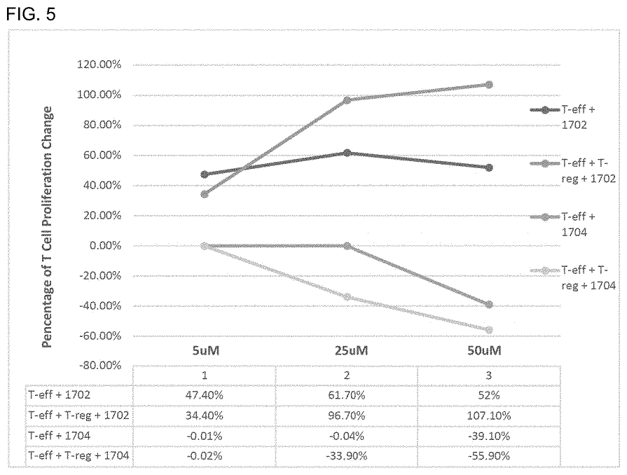

Antigen specific activated T cells and target cancer stem cell lines that are HLA A1 and A2 positive are generated. Cancer stem cell lines are tested for HLA to obtain target cells for in vitro killing assays. Small molecule agonist of the glucocorticoid induced TNF like receptor such as RMGL171102 to enable both in vitro and in vivo propagation of the activated T cells is also tested. This molecule is highly active in increasing effector T cell propagation and Treg inhibition (FIG. 5).

The presently claimed invention also includes use of the compound of Formula I such as RMGL 171102 (aka, molecule or compound 11702) as an effector T eff proliferation agent.

The inventive dendritic cell-based immunotherapy creates a dendritic cell ex vivo, using the patient's own white blood cells which, when reintroduced into the patient's body, are programmed to find the cytotoxic T cells and have them target the cancer and kill cancer cells. In contrast, based on the G-Rex gas permeable T cell propagation technology, T cells are propagated in vitro (both cytotoxic T cells and helper T cells). These cells are harvested from the patient, which are then presented with specific cytotoxic and helper antigens by dendritic cells outside of the patient's body. When these activated T cells are reintroduced, they divide as antigen-specific killer T cells and helper T cells that help cytotoxic T cell propagation. An important component of the development of this therapy is the identification and selection of antigens that are able to generate activated T cells that then recognize cancer stem cells to kill it. The generation of activated antigen-specific T cells outside of the patient's body, enables antigen presentation of dendritic cells to T cells to occur outside the immunosuppressive milieu of a cancer patient's body. It enables the generation of activated T cells and expansion of these T cells without the immunosuppressive systemic influence of the cancer and of immunosuppressive therapies such as radiation therapy and chemotherapy.

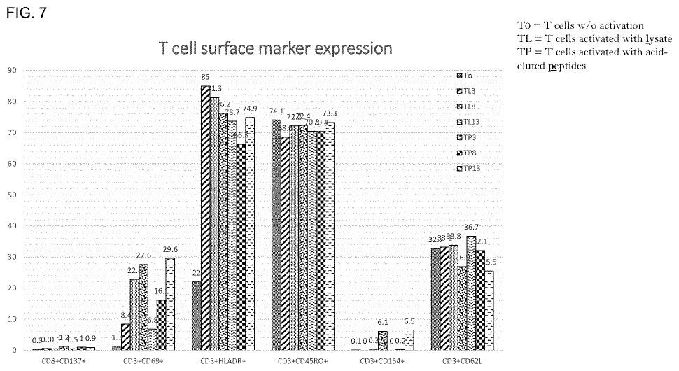

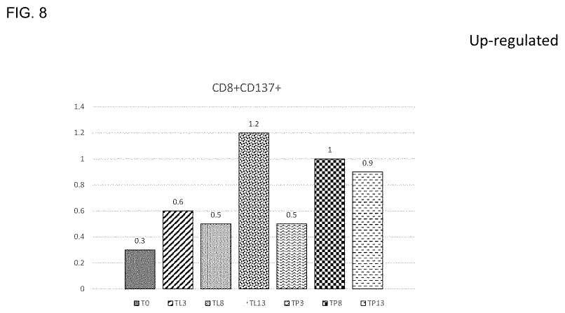

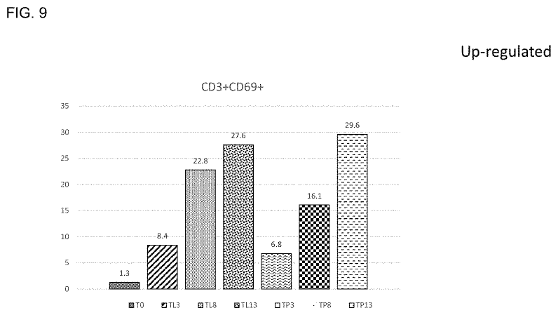

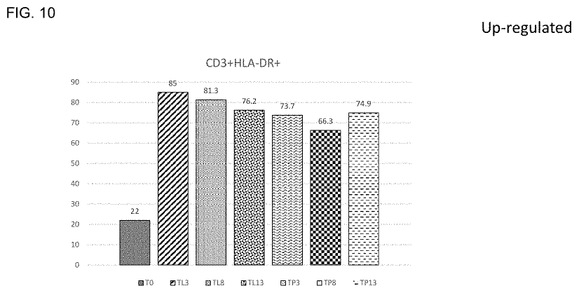

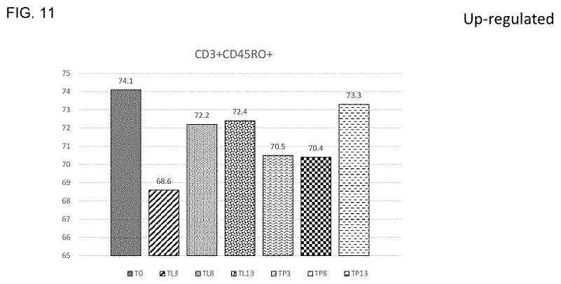

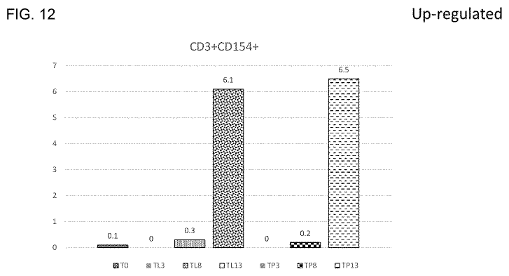

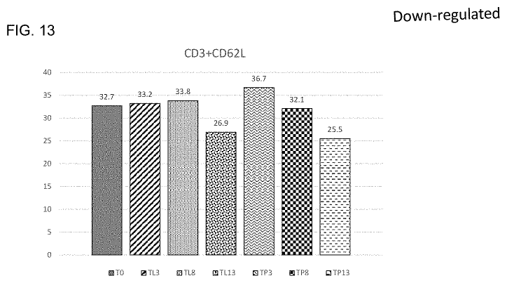

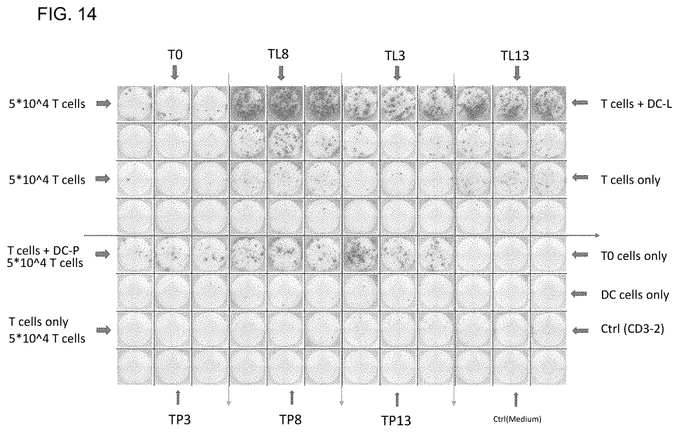

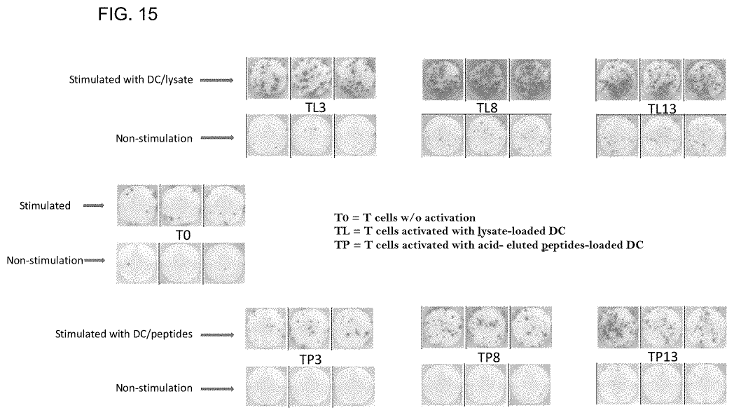

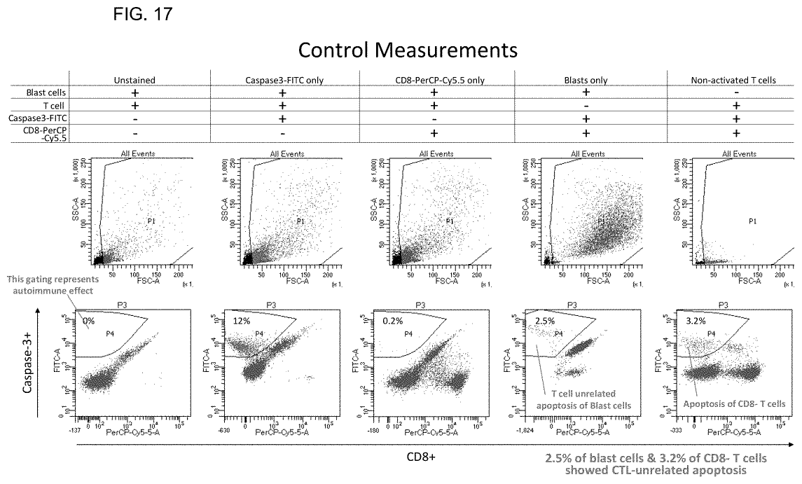

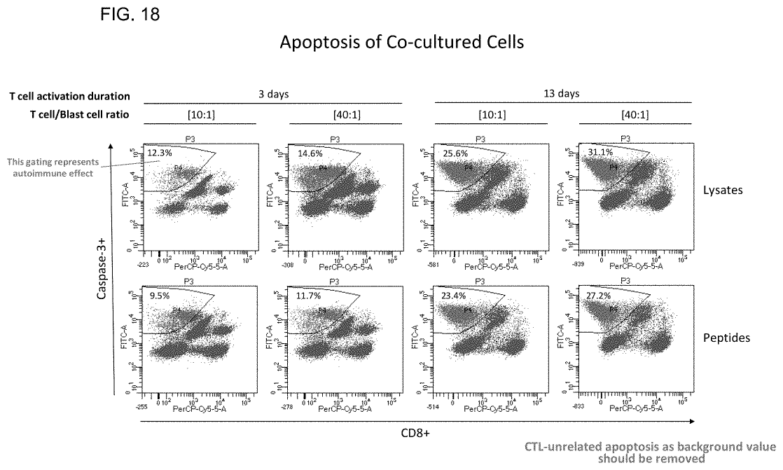

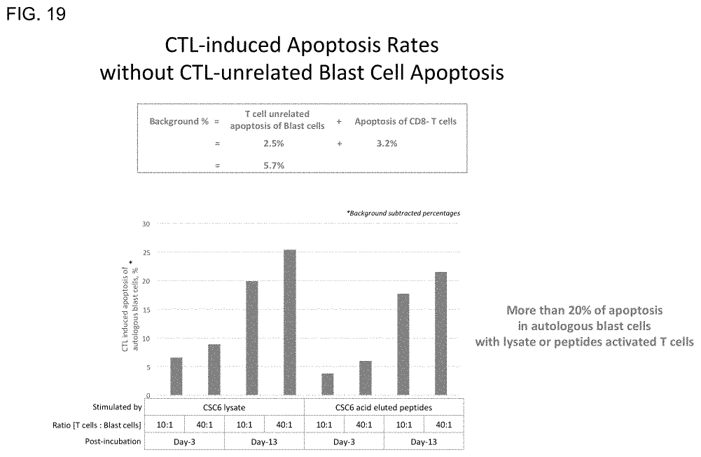

When both tumor lysate or MHC1 antigens were used, all activation surface markers (CD137, CD69, CD45RO, CD154, HLA-DR CD62L) show up-regulation or down-regulation as expected. Both CSC6 lysate and CSC6 acid-eluted peptides create similar activation responses in T cells, but lysate shows much stronger stimulation effect. T cells expand much faster after Day8 (with addition of IL-2), and Day13 T cells show higher expression of most activation markers. All stimulated groups show more than 3-fold increase of IFN-γ secretion compared to non-stimulated groups. T cells stimulated by lysate for 8 & 13 days show the greatest amount of IFN-γ spots compared to other groups (FIGS. 7-16). Autoimmune effect tends to be higher as cytotoxicity goes up. About 20% of CTL-induced apoptosis is high. However there was no autoimmune cytotoxicity than control in all tested assays of CTL against self PHA blasts. (FIGS. 17-19)

The present invention can also be practiced to treat inflammatory conditions, such as autoimmune disorders by contacting T cells with antigen presenting cells bearing inflammatory condition antigen, such as autoimmune disease antigen ex vivo, such as multiple sclerosis. The T cells may be stimulated dendritic cells, with autoimmune antigens such as myelin associated proteins. Optionally, GITR/GITRL antagonist may be employed ex vivo to the sample of T cells to create further modified T cells. The GITR/GITRL antagonist compound may be RMGL 171104.

In further detail, with regard to the above-described CTL (Cytotoxic T Lymphocyte) and helper antigens and their epitope peptides that are used in the present invention, in one aspect, the following protocol may be used.

The manufacture of activated T cells starts with apheresis of a patient at the Blood Donor Facility (BDF). Monocytes and Lymphocytes are enriched from the apheresis product using elutriation with the Elutra®. Lymphocyte collection is cryopreserved until Day 6. Monocytes are seeded into MACS Cell Differentiation Bags® in CellGenix DC media supplemented with 100 ng/ml of GM-CSF and 34.5 ng/mL of IL-4. On Day 4 of culture, LPS (final concentration of 60 EU/ml) and IFN-γ (final concentration of 2000 IU/ml are added to culture bags for DC maturation. On Day 5 of culture, 20-24 hours after LPS and IFN-γ addition, 20 ug/ml of each GBM cancer stem cell epitope (18 in total in the present exemplification) is added to DCs to pulse together for another 16-20 hours. On Day 6 of culture, DC is harvested, washed, counted and checked for quality by Flow Cytometry. Qualified DC will be mixed with Lymphocytes cryopreserved at Day 0 which has been thawed and recovered for 2 hours early on Day 6, at DC:T cell ratio of 1:10. Cell mixture is placed in a G-Rex® 100 container with T Cell Culture Medium supplemented by IL-4 (34.5 ng/ml) and IL-7 (10 ng/ml) for activation and rapid expanding. IL-2 is added into culture on day 8 at final concentration of 40 U/ml and is replenished every 2-3 days. 75% of culture media will be replaced with fresh media along with replenishment of IL-4 and IL-7 every 5 days. Cells remain in G-Rex® container for 12-day growth before the desired number of cells is reached. The entire process takes 18 days after which T cells activated by epitope-pulsed DC are harvested, checked for sterility and quality, and cryopreserved.

The present invention is directed to a method of using activated T cells (ATC) autologous cellular product to treat patients with cancer, in particular, recurrent glioblastoma, and further in particular, recurrent glioblastoma multiform (GBM) in HLA-A2 patients and HLA-A1 patients. The present invention is directed to using autologous T cells activated with autologous dendritic cells loaded with cancer-associated antigens, in particular, as exemplified in the present application, glioma-associated antigens. The activated T cell product may be formulated as a cellular product.

In one aspect, the activated autologous T cells may be administered intravenously, and dosing may be without limitation preferably once.

ATC therapy is an autologous activated T cell therapy. Additionally, only immune cells of certain specific HLA haplotypes are able to recognize and mount an immune response to the antigens presented on the autologous dendritic cells. Thus, traditional toxicology or pharmacology studies with the autologous cellular product are not possible. The preclinical data relevant to autologous activated T cells consists of data on the selection of the peptides used for pulsing DC and generating autologous T cells. This includes data on the presence of peptide-related antigens present on GBM tumor cells and the ability of these peptides to stimulate cytotoxic T cell responses.

ATC is an Autologous T Cell Therapy with Selectivity to Specific HLA Haplotypes.

Both naïve T cells and monocytes are obtained from patients/donors' leukapheresis product through elutriation. Monocytes are differentiated into mature dendritic cells induced by interferon-gamma and LPS. The process of DC maturation is completed by pulsing the cells with cancer associated epitopes. Matured DCs are then presented to naïve T cells and co-cultured in G-Rex container for rapid growth. During the process, T cell surface markers for activation are detected. An Elispot assay was performed on the final product to measure the Interferon-gamma secretion towards specific tumor antigen or against cancer stem cell lines.

The specificity to the human immune response and to specific HLA haplotypes indicates that traditional toxicology or pharmacology studies with ATC are not possible. The nonclinical data consists of data on the selection of the peptides used for pulsing DC (based on their presence on GBM tumor cells, for instance) and the ability of these peptides to stimulate cytotoxic T cell responses.

Selection of Peptides for ATC

Success in immunotherapeutic approaches for cancer therapy depends upon efficient activation of reactive T lymphocytes, and of activated T lymphocytes (CTL) in particular. T cells become activated by interaction with antigen-presenting cells (APC). DC, which are derived from bone marrow or peripheral blood mononuclear cells, are the most potent professional APC's in the body. The objective is to pulse autologous, peripheral blood DC with both cytotoxic and helper tumor peptides to generate activated cytotoxic and helper T cells and reinject them into the patient. The cellular immune response should then go to the intracranial tumor and potentially generate a long-lived cytotoxic response.

In order to determine which tumor cell antigens might be useful, the presence of mRNA and protein expression in 43 primary GBM cell lines and seven established human GBM cell lines were characterized in a study (20). HER-2, gp100, and MAGE1 mRNA expression was detected in 81.4%, 46.5%, and 39.5% of the GBM primary cell lines, respectively. Using immunoreactive staining analysis by flow cytometry, HER-2, gp100, and MAGE1 protein expression was detected in 76%, 45%, and 38% of the GBM primary cell lines, respectively. These data indicate that HER-2, gp100, and MAGE1 could be used as tumor antigens to pulse DC and develop antigen-specific active immunotherapy strategies for GBM patients (2).

AIM2 antigen is expressed in a wide variety of tumor types so its expression was analyzed in GBM in primary cultured cells and established GBM cell lines (21, 22). Primary GBM cell lines expressed 88.4% and 93.0% of non-spliced and spliced AIM2, respectively. Five out of seven of the established GBM cell lines expressed both non-spliced and spliced AIM2. A CTL clone that was specific for AIM2 peptide, recognized GBM tumor cells as determined by interferon-gamma release. These data indicate that AIM2 could be used as a tumor antigen target to develop antigen specific active immunotherapy for glioma patients (3). AIM-2 antigen is isolated from immunoselected melanoma-2 cDNA clone that generated a peptide that was encoded within a short open reading frame of 23 amino acids and conforming to the HLA-A1 binding motif RSDSGQQARY.

The addition of the antigen TRP-2 to the mix of antigens used to pulse DC may also be an advantage. TRP-2 was present in 51% of primary tumor cell lines derived from patients with glioblastoma multiforme (GBM) and in vitro generated T cells that specifically lysed T2 cells pulsed with TRP-2 peptide and TRP-2 positive GBM cell lines (23). TRP-2 can induce specific CTL activity in patients who received immunotherapy with tumor lysate-pulsed DC. Furthermore TRP-2 expression in two patients' recurrent tumor cell lines was significantly decreased which might be explained by the observation that TRP-2 over-expression significantly increased resistance to chemotherapy. Immunological targeting of tumor-associated antigen TRP-2 might increase sensitivity to chemotherapy (4).

IL-13Rα2 is a glioma-restricted receptor for interleukin-13 (24) and has also been identified as a possible target for immune stimulation (25, 26). It is expressed at low levels in low-grade astrocytomas and its expression increases with the progression to higher-grade malignancy (5). Thus, IL-13Rα2 is a potential target for therapeutic intervention with immune therapy.

GBM tumors were evaluated for antigen expression by the measurement of mRNA by PCR. As shown in Table 11, expression of three or more antigens was observed on all of the patient tumors, expression of 4 or more antigens on 97% of tumors and expression of 5 or more antigens on 93% of tumors. Expression of all six antigens was observed in 83% of patient tumors. The highest expression was observed for AIM2, TRP-2, HER2 and IL-13Rα2 with MAGE1 and gp100 showing weaker expression. Together these studies demonstrate significant expression of these antigens on GBM tumors, making them good candidates for immunotherapy targets.

EphA2 belongs to the ephrin receptor subfamily of the protein-tyrosine kinase family. EPH and EPH-related receptors have been implicated in mediating developmental events, particularly in the nervous system. Receptors in the EPH subfamily typically have a single kinase domain and an extracellular region containing a Cys-rich domain and two fibronectin type III repeats. The ephrin receptors are divided into 2 groups based on the similarity of their extracellular domain sequences and their affinities for binding ephrin-A and ephrin-B ligands. EphA2 binds ephrin-A ligands and is a transcriptional target of the Ras-MAPK pathway. It is thought to play a role in tumor cell invasion by regulating integrins and focal adhesion kinase (FAK) dephosphorylation.

EphA2883-891 peptide was incorporated in polypeptide vaccines to safely induce antigen specific immune responses in pediatric patients with gliomas including diffuse pontine gliomas (6-9).

EphA2883-891 peptide (TLADFDPRV), which has previously been reported to induce interferon-gamma in HLA-A2+ PBMCs. Stimulated PBMCs demonstrated antigen-specific cytotoxic T lymphocyte (CTL) responses as detected by specific lysis of T2 cells loaded with the EphA2883 peptide as well as HLA-A2+ glioma cells, SNB19 and U251, that express EphA2. Furthermore, in vivo immunization of HLA-A2 transgenic HHD mice with the EphA2883-891 peptide resulted in the development of an epitope-specific CTL response in splenocytes, despite the fact that EphA2883-891 is an autoantigen in these mice (6-9).

MAGE-A1 was shown to be expressed on 64% of glioblastomas. One of the predicted epitopes, MAGE-A1(278-286)(KVLEYVIKV), was found to be presented by HLA-A*0201, with an estimated copy number of 18 molecules/cell. HLA-A*0201 transgenic mice (HHD mice) were used to generate CTL lines that stained positive with an HLA-A*0201 tetramer folded around the KVLEYVIKV peptide and killed peptide-loaded mouse target cells expressing HLA-A*0201. IFN-gamma-treated or -nontreated HLA-A*0201 expressing HeLa cells transiently transfected with a plasmid expressing the MAGE-A1 gene stimulated in vitro cytokine production by the CTL lines. Moreover, IFN-gamma-treated KS24.22 cells, but not IFN-gamma-treated HLA-A*0201(+) MAGE-A1(−) cells or IFN-gamma-treated HLA-A*0201(−) MAGE-A1(+) cells, were killed by these cytotoxic T cells.

MAGE-A1 was utilized as part of a multi-epitope vaccine in a DC-based phase I trial for high grade glioma. All patients demonstrated MAGE-A1 expression and developed CTL immune responses against the antigen. All 76 DC injections were well tolerated except for transient liver dysfunction with grade II (10).

Although NY-ESO-1 is expressed in normal adult tissues solely in the testicular germ cells of normal adults, it is expressed in various cancers including glioblastoma, melanoma, lung, breast, and ovarian cancers.

Treatment of intracranial glioma-bearing mice with decitabine reliably and consistently induced the expression of an immunogenic tumor-rejection antigen, NY-ESO-1, specifically in glioma cells and not in normal brain tissue. The upregulation of NY-ESO-1 by intracranial gliomas was associated with the migration of adoptively transferred NY-ESO-1-specific lymphocytes along white matter tracts to these tumors in the brain. Similarly, NY-ESO-1-specific adoptive T cell therapy demonstrated antitumor activity after decitabine treatment and conferred a highly significant survival benefit to mice bearing established intracranial human glioma xenografts. Transfer of NY-ESO-1-specific T cells systemically was superior to intracranial administration and resulted in significantly extended and long-term survival of animals.

Twenty-eight out of 38 GBM specimens tested positive for NY-ESO-1 IFN-γ production in NY-ESO-1+-sorted T-cells showed that NY-ESO-1-peptide-expanded T-cells were able to react against naturally processed and presented peptides on HLA-A2⁺ tumor cell lines.

Antigen-specific IFN-γ responses in 25% blood samples for NY-ESO-1. NY-ESO-1-expanded T-cells recognized naturally processed and presented epitopes including SLLMWITQC (11).

Helper Peptides

Helper T lymphocyte (HTL) epitopes increase the CTL precursor frequency to CTL epitopes, which last more than a year after vaccination. Antigen-specific HTLs may prolong CTL responses. Helper peptides were chosen to complement CTL peptides. Several were chosen for their promiscuous property of being recognized by multiple HLA subtypes allowing their ubiquitous use in multiple HLA contexts.

HER 883 is a promiscuous MHC Class II helper T cell epitope. A predictive algorithm described by Southwood et al. to identify promiscuous HLA-DR binding peptides was used to identify this epitope. It was recognized by T cells in context of HLA-DR1, HLA-DR4, HLA-DR52, and HLA-DR53, indicating a high degree of histocompatibility promiscuity (12).

HER2/neu 369, HER2/Neu 671, and HER2/neu 688 are helper peptides containing HLA-A2 binding motifs. Vaccination in breast cancer patients led to peptide specific T cells that were able to lyse tumors and led to long lived immune responses in select patients (13).

TRP-2 60-74 and TRP-2 149-163. TRP-2 immunized mice developed CD4+ T cell reactivity against the known HLA-DRB1*0301-restricted TRP-2(60-74) epitope and against the new epitope TRP-2(149-163). TRP-2(149-163)-responsive T cells were obtained from healthy individuals, and in vitro stimulation of PBMC revealed the presence of epitope-reactive CD4+ T cells in melanoma patients (14).

Gp100 576-590

Helper peptide restricted by HLA-DR7. Goal was to identify promiscuous MHC class II helper T lymphocyte epitopes for gp100 (15).

EphA2 663-667

Recognized most by T cells DR4+ donors Glioma EphA2 antigen (16, 17)

NY-ESO-1 119-143

Promiscuous class II region containing epitopes that bind to multiple HLA-DR alleles. Naturally processed by APC or naturally presented by tumor cell lines. Induced NY-ESO-1 specific CTL in NY-ESO-1 seropositive epithelial ovarian cancer patients. This epitope demonstrated dual HLA class I and II specificities (18, 19).

Developing an activated T cell immunotherapeutic approach to treat glioblastoma

We use two types of antigens: MHC Class I antigens including gp100, MAGE-1, MAGE-A1, TRP2, HER-2, AIM-2, IL-13 Rec alpha2, EphA2, NY-ESO-1 and nine related MHC Class II antigens. The expression of these tumor antigens in glioblastoma and their evident immunogenicity make them useful targets for CTL therapy. Broad-spectrum antigen-specific T cells targeting multiple antigens for all patients can be generated by these two types of antigens activating both CD4⁺ and CD8⁺ T cells.

It is contemplated herein that the cytotoxic and helper antigens as identified herein may be used. A full protein antigen or an epitopic peptide of the antigen is envisioned. The peptide to be pulsed may be between 8 to about 40 amino acids long, or 8 to about 30, 8 to 25 to or 8 to 22 or about 8 to 14 amino acids long so long as the antigen whole protein, or its epitopic fragment thereof are able to provide activation to the T cell.

In particular, many of the above-described antigens and polypeptides derive from the melanoma tumor which are derived embryologically from the neural crest which is the origin of glial tumors, particularly glioblastoma. Many of the above described antigens are up regulated in cancer stem cells, in particular glioblastoma stem cells.

Protocol

Blood Procurement for CTL and Antigen-Presenting Cell (APC) Generation

Generation of tumor-specific CTL lines requires the generation of several different components from peripheral blood mononuclear cells (PBMC) The CTL line may be derived from patients' peripheral blood T cells, by stimulation with antigen-presenting cells (APCs) pulsed with tumor antigens as listed of MHC I and II. Fresh peripheral blood mononuclear cells (PBMC) are isolated by Leukapheresis and then are separated into several fractions by Elutriation. Lymphocyte fraction is cryopreserved for later CTL line manufacture while Monocyte fraction is differentiated into Dendritic Cells (DCs) for antigen-presenting.

To initiate tumor-specific CTL lines, we generate DCs (“stimulator”) by culture of PBMC-derived Monocytes with cytokines (GM-CSF 100 ng/ml, IL-4 34.5 ng/ml) for 4 days followed by maturation with a standard DC maturation cocktail (IFN-gamma 2000 U/ml and LPS 60 EU/ml) overnight. These matured Dendritic Cells are pulsed for 16-20 hours with tumor associated antigens such that the final concentration of each peptide is 20 ug/ml. Subsequently the DCs are washed once and used to stimulate PBMC-derived Lymphocytes (Elutriation fraction) at responder:stimulator (R:S) ratio of 10:1. For initiation, at least 2×10⁸ Monocytes are seeded to generate enough DCs at a yield rate of 1/3 In the end of culture DC surface marker is checked to make sure the quality meets the criteria for further T cell activation.

Cryopreserved Lymphocytes from Elutriation are thawed, washed, and recovered in plain T cell culture medium for 2 hours. Then these “responder” cells will be checked for viability as well as the total number to ensure the appropriate R:S ratio before being mixed with peptide-pulsed DCs.

To expand the antigen-specific T cells, we use Wilson Wolf's G-Rex container for rapid growth and proliferation. G-Rex has a unique Gas Permeable Membrane to facility O₂ and CO₂ exchange between culture compartment and ambient air. 1-2×10⁸ T cells are seeded along with peptide-pulsed DCs in each container at proper ratio. Two containers will be applied if needed to generate sufficient amount of activated T cells.

Cell mixture is transferred into G-Rex after resuspension in T cell culture medium enhanced with IL-4 (34.5 ng/ml) and IL-7 (10 ng/ml) at concentration of 1×10⁶/ml. Two days later (day 8 of whole process) three quarters of culture medium is replaced by fresh medium, replenished IL-4, IL-7, and addition of IL-2 (40 U/ml). Medium change happens every 5 days of culture with refreshed IL-4 and IL-7. IL-2 shall be replenished every other day. Cell growth is monitored by cell counting and viability check.

At the end of the culture period (day 18), CTLs are harvested, formalized into final product by cryopreservation at 1.2×10⁸ cells/vial and stored in Liquid Nitrogen. Aliquots are taken from final product to test for function, specificity, identity, and sterility. The frequency of tumor-specific CTLs is determined using Interferon-gamma secretion staining (ELIspot assay). Effector memory phenotype and T cell subsets are analyzed by Flow Cytometry.

Infectious disease testing and HLA identity may be performed within 7 days of blood collection as an enrollment requirement. Release criteria includes T cell identity, viability >70%, negative culture for bacteria and fungi after 7 days, endotoxin testing <5 EU/ml, negative result for Mycoplasma, <10% killing of patient PHA blasts at 20:1 ratio (if an allogeneic product).

On day of treatment, cryopreserved vial is thawed in 37° C. water bath. Cells are washed, passed through 40 μm cell strainer to eliminate cell clusters. Viable cell is counted then resuspended in infusible medium prior to delivery to clinic.

At first, HLA-A2 positive donor's autologous Monocytes enriched by Elutriation were differentiated into Dendritic Cells with GM-CSF and IL-4 and matured by LPS plus Interferon-gamma. Then a pool of 9 CTL epitopes (final concentration 20 ug/ml of each) and 9 Helper epitopes (final concentration 20 ug/ml of each) were pulsed with DCs for 18 hours to complete maturation before mixing with naïve T cells at 1:10 ratio in G-Rex container for 12-day culture. Medium, cytokine IL-4, IL-7 and IL-2 were replenished as needed during the process. At day 12, T cells were sampled for surface marker staining and Interferon-gamma secretion measurement. T cells from two groups were compared in Elispot assay while targeting two types of T2 cells (epitope-loaded T2 vs. no-epitope T2) and four institute-established GBM cancer stem cell lines CSC38b, CSC40b, CSC59 and CSC66. These CSC lines are all HLA-A2 positive.

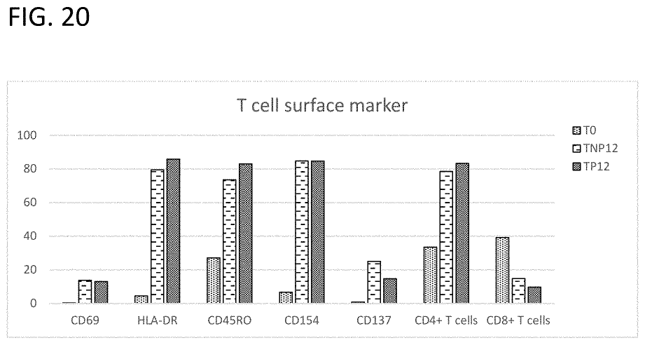

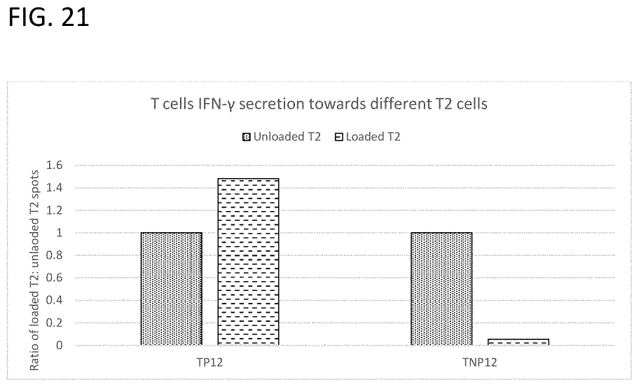

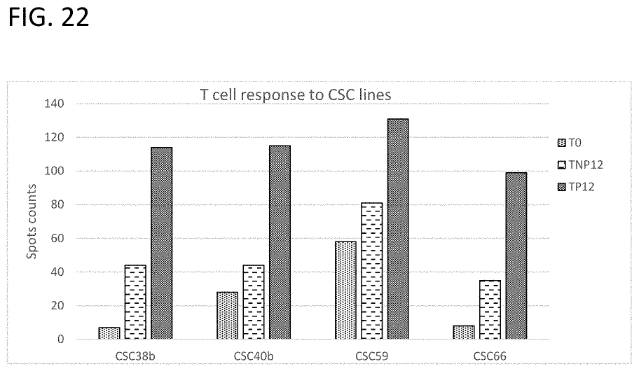

After 12 days culture, all T cell activation surface markers were upregulated while CD4+ population increased and CD8+ population decreased following T cell proliferation and expansion (FIG. 20). Elispot data not only show that activated T cells secrete much more Interferon-gamma than naïve T cells (T0), but further indicates that T cells activated by epitope-pulsed DCs (TP) specifically recognize the pooled epitopes loaded onto the T2 cells as compared to unloaded T2 cells. Similar results were seen when we use CSC line cells as Elispot targets. (FIGS. 21, 22)

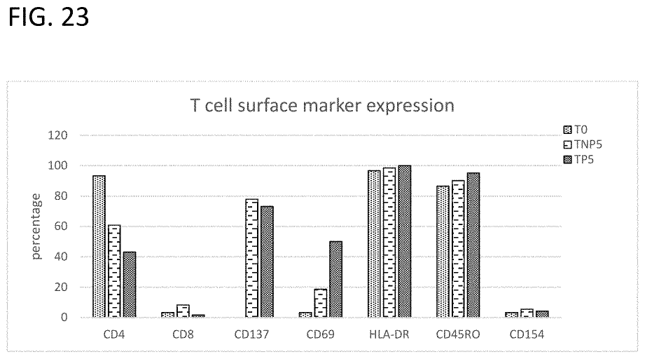

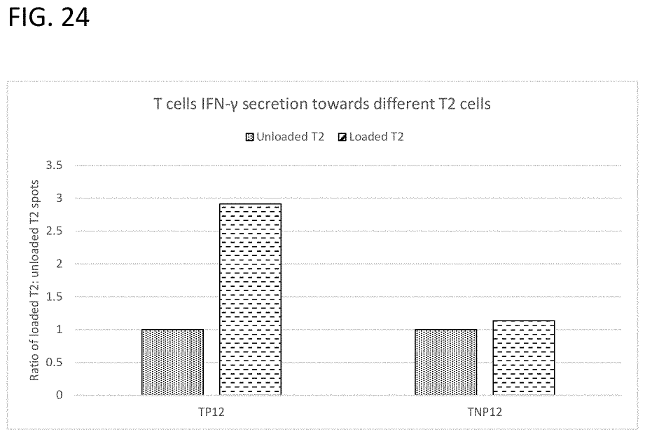

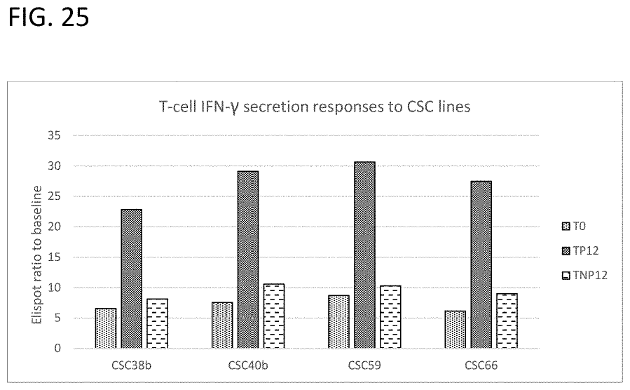

We repeated the assay wherein T cells were sampled on day 5 for surface marker staining to see how soon these cells can be activated. Elispot assay was performed after 12-day expansion in G-Rex container. The results show T cells express activation markers as early as 5 days in culture (FIG. 23). Interferon-gamma secretion outcome confirms that activated T cells are effective targeting epitope-loaded T2 as compared to naïve T cells (T0), and there is significant difference between T cells activated by epitope-pulsed DCs (TP) and those activated by no-epitope DCs (FIGS. 24, 25).

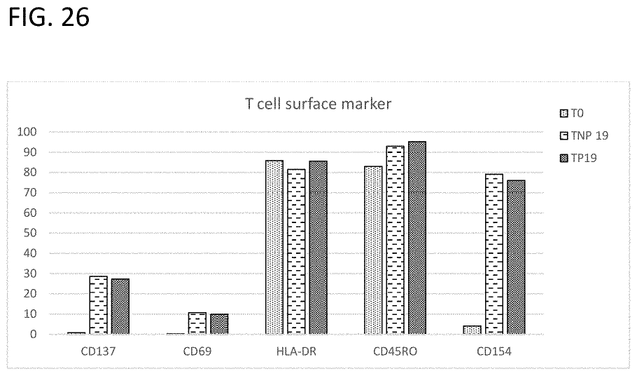

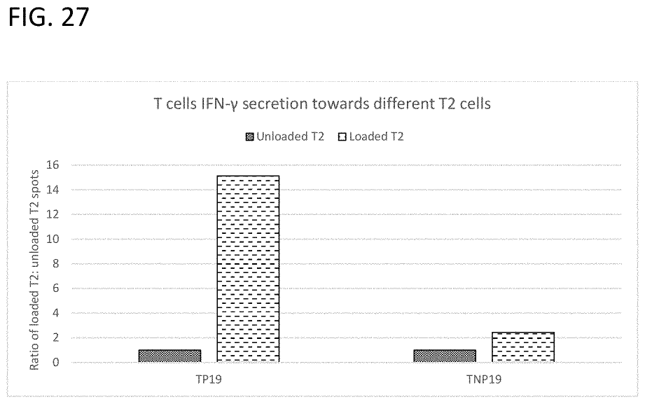

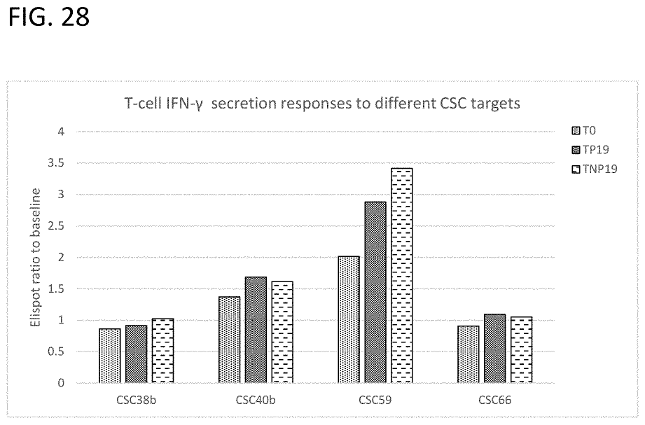

In further studies, we extended the T cell culture period to 19 days to optimize maximum growth of the T cell population. Activation markers and Interferon-gamma secretion of T cells were studied. Our data show, while possessing the same tumor antigen-specific immune responses and enhanced Interferon-gamma secreting reaction towards epitope-loaded T2 cells and cancer stem cells, no further advantage was noted in prolonged 19-day-culture compared to 12-day T cells. (FIGS. 26, 27, 28)

Cytotoxicity of Activated T Cells to Cancer Stem Cells

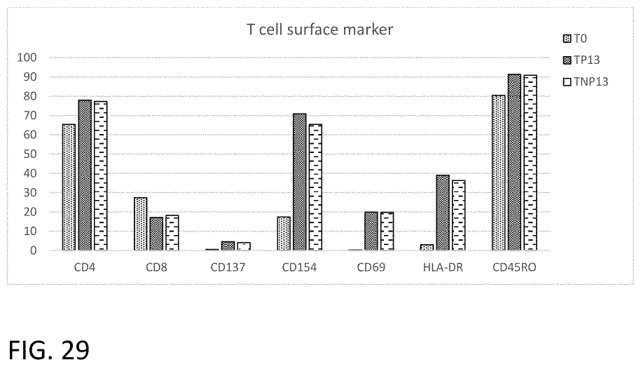

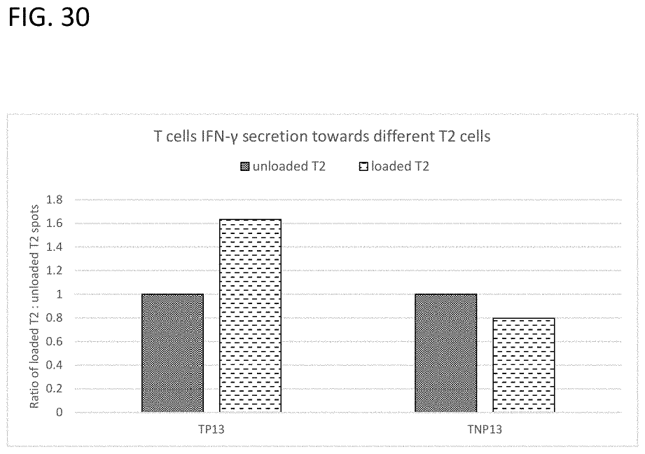

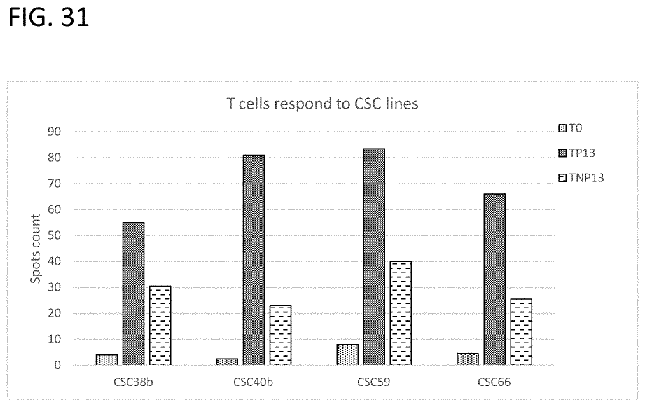

In most recent repeat of experiment (Assay #8), we stimulated T cells with both epitope-pulsed DCs (TP13) and no-epitope DCs (TNP13) and expanded them in G-Rex container for 13 days. Cell surface activation markers as well as Elispot assay targeting different types of T2 cell were studied. All surface markers including CD69, CD137, CD153, CD45RO and HLA-DR are upregulated as seen many times before. TP13 secretes more Interferon-gamma towards peptide-loaded T2 cells as compared to unloaded T2 cells. The difference is significant as to confirm the antigen-specific killing effect of TP13. There is no such difference when T cells were activated by no-epitope DCs (TNP13). We also repeated the Elispot assay using institute-established lines CSC38b, CSC40b, CSC59, and CSC66 as targets to study the efficiency of two types of T cells responding to GBM cancer stem cells. Result show both T cells (TP13, TNP13) have increased Interferon-gamma secretion against these cell lines compared to naïve T cells (T0), but TP13 demonstrates much stronger cytotoxicity than TNP13. (FIG. 29, 30, 31)

Testing for Autoimmune Response

To determine whether activated T cells against glioblastoma antigens may induce an autoimmune response, PHA blasts are generated from the PBMC of cells that were used to generate the activated T cells. Then the activated T cells are mixed with the PHA blasts to determine the percentage killing of the PHA blasts as a surrogate of potential autoimmune responses.

### Experiment 1

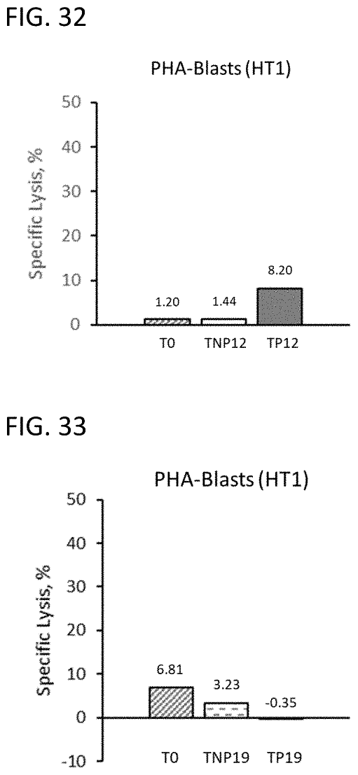

PHA-blasts were generated by stimulating PBMC (donor: HT1) with PHA. E:T co-culture ratio was 5:1. In this assay, CFSE was used to stain PHA-blasts. Plot in FIG. 32 shows that less than 10% of PHA-blasts were killed by any of T cells (T0, TNP12 or TP12).

TABLE 2
Condition of assay.

| | |
|---|---|
| T cell activation | Peptides, 12 days |
| Target dye | CFSE |
| Live/Dead dye | eFluor 780 |
| E:T ratio | 5:1 |
| Incubation | 4 hours |
| Analysis | Fortessa Flow Cytometry |

TABLE 3
Raw data and calculated % specific lysis.

| | Dead PHA-blasts, % | Specific Lysis, % |
|---|---|---|
| BL only | 6.29 | — |
| BL + T0 | 7.41 | 1.20 |
| BL + TNP12 | 7.64 | 1.44 |
| BL + TP12 | 13.97 | 8.20 |

### Experiment 2

PHA-blasts were generated by stimulating PBMC (donor: HT1) with PHA. E:T co-culture ratio was 5:1. In this assay, CFSE was used to stain PHA-blasts. Plot in FIG. 33 shows that less than 10% of PHA-blasts were killed by any of T cells (T0, TNP19 or TP19).

TABLE 4
Condition of assay.

| | |
|---|---|
| T cell activation | Peptides, 19 days |
| Target dye | CFSE |
| Live/Dead dye | eFluor 780 |
| E:T ratio | 5:1 |
| Incubation | 4 hours |
| Analysis | Fortessa Flow Cytometry |

TABLE 5
Raw data and calculated % specific lysis.

| | Dead PHA-blasts, % | Specific Lysis, % |
|---|---|---|
| BL only | 13.3 | — |
| BL + T0 | 19.2 | 6.81 |
| BL + TNP19 | 16.1 | 3.23 |
| BL + TP19 | 13.0 | −0.35 |

### Experiment 3

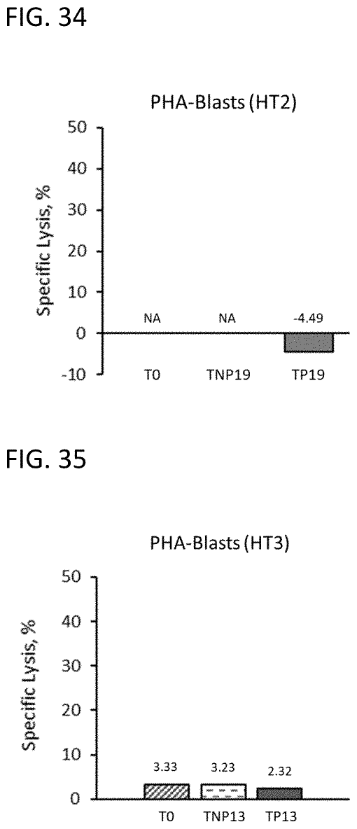

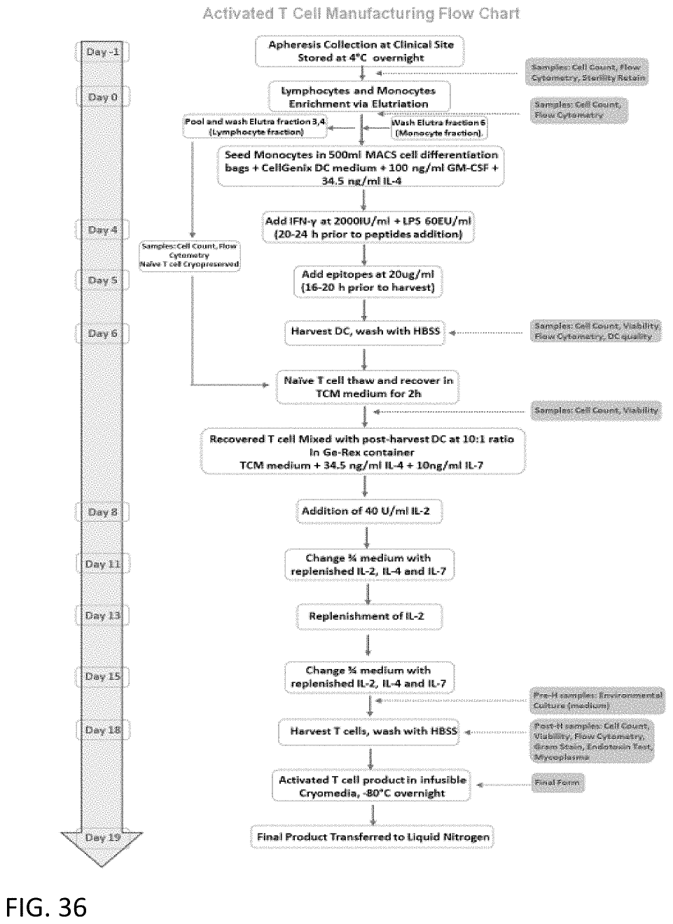

PHA-blasts were generated by stimulating PBMC (donor: HT2) with PHA. E:T co-culture ratio was 1:10. In this assay there was no T0 and TNP samples because it was testing CellTrace Violet (CTV) dye. However, the percentage of dead PHA-blasts, which was co-cultured with activated T cells (TP19), was very small (Table 7 and FIG. 34).

TABLE 6
Condition of assay.

| | |
|---|---|
| T cell activation | Peptides, 19 days |
| Target dye | CellTrace Violet (CTV) |
| Live/Dead dye | eFluor 780 |
| E:T ratio | 1:10 |
| Incubation | 4 hours |
| Analysis | Fortessa Flow Cytometry |

TABLE 7
Raw data and calculated % specific lysis.

| | Dead PHA-blasts, % | Specific Lysis, % |
|---|---|---|
| BL only | 8.6 | — |
| BL + T0 | NA | NA |
| BL + TNP19 | NA | NA |
| BL + TP19 | 4.5 | −4.49 |

### Experiment 4

PHA-blasts were generated by stimulating PBMC (donor: HT3) with PHA. E:T co-culture ratio was 20:1. Plot in FIG. 35 shows that less than 5% of PHA-blasts were killed by any of T cells (T0, TNP13 or TP13). There was no significant effect of activated T cells on allogenic immune cells.

TABLE 8
Condition of assay.

| | |
|---|---|
| T cell activation | Peptides, 13 days |
| Target dye | CellTrace Violet (CTV) |
| Live/Dead dye | eFluor 780 |
| E:T ratio | 20:1 |
| Incubation | 4 hours |
| Analysis | Fortessa Flow Cytometry |

TABLE 9
Raw data and calculated % specific lysis.

| | Dead PHA-blasts, % | Specific Lysis, % |
|---|---|---|
| BL only | 0.8 | — |
| BL + T0 | 4.1 | 3.33 |
| BL + TNP13 | 4.0 | 3.23 |
| BL + TP13 | 3.1 | 2.32 |

The peptides used to pulse DC for T Cell Activation are manufactured by PolyPeptide Laboratories and include nine-MHC class I and nine-MHC class II peptides (Table 10). These synthetic peptides were selected based on epitopes present on GBM tumor cells and some of which are also over expressed on the cell surface of target cancer stem cells. The goal is to induce a specific anti-tumor T cell response to GBM.

TABLE 10
Peptides used in preparation of DCs

| Type | Antigen | Epitopes | Sequence |
|---|---|---|---|
| MHC I | MAGE1 | | H-EADPTGHSY-OH |
| | | | (SEQ ID NO: 7) |
| | AIM2 | | H-RSDSGQQARY-OH |
| | | | (SEQ ID NO: 11) |
| | TRP-2 | | H-SVYDFFVWL-OH |
| | | | (SEQ ID NO: 9) |
| | gp100 | | H-IMDQVPFSV-OH |
| | | | (SEQ ID NO: 6) |
| | HER2 | | H-VMAGVGSPYV-OH |
| | | | (SEQ ID NO: 12) |
| | IL-13Rα2 | | H-WLPFGFILI-OH |
| | | | (SEQ ID NO: 13) |
| | MAGE-A1 | | H-KVLEYVIKV-OH |
| | | | (SEQ ID NO: 14) |
| | EphA2 | | H-TLADFDPRV-OH |
| | | | (SEQ ID NO: 10) |
| | NY-ESO-1 | | H-SLLMWITQC-OH |
| | | | (SEQ ID NO: 8) |
| MHC II | HER2/neu 369-384 | | H-KIFGSLAFLPESF |
| | | | DGDPA-OH (SEQ |
| | | | ID NO: 21) |
| | NY-ESO-1 119-143 | | H-PGVLLKEFTVSGN |
| | | | ILTIRLTAADHR-OH |
| | | | (SEQ ID NO: 16) |
| | TRP-2 149-163 | | H-KKRVHPDYVITTQ |
| | | | HWL-OH (SEQ ID |
| | | | NO: 18) |
| | HER 883 | | H-KVPIKWMALESIL |
| | | | RRRF-OH (SEQ ID |
| | | | NO: 20) |
| | HER/neu 668-703 | | H-RRLLQETELVEPL |
| | | | TPS-OH (SEQ ID |
| | | | NO: 22) |
| | EphA2 663-667 | | H-EAGIMGQFSHHNI |
| | | | IR-OH (SEQ ID |
| | | | NO: 19) |
| | HER/neu 671-684 | | H-ELVSEFSRMARDP |
| | | | Q-OH (SEQ ID |
| | | | NO: 23) |
| | Gp100 576-590 | | H-SLAVVSTQLIMPG |
| | | | QE-OH(SEQ ID |
| | | | NO: 15) |
| | TRP-2 60-74 | | H-QCTEVRADTRPWS |
| | | | GP-OH(SEQ ID |
| | | | NO: 17) |

TABLE 11
In Process Tests for T Cell Activation

| Day of | | | | |
|---|---|---|---|---|
| Manufacturing | Sample | Parameter | Test Method | Data Reported |
| Day 0 | Apheresis product | Cell Count | CBC | cells/mL |
| Day 0 | Post Elutra Culture | Cell Count | CBC | cells/mL |
| | count | | | |
| Day 0 | Lymphocyte | % Lymphocyte | Flow | % of CD3+, |
| | fraction | | Cytometry | CD4+, CD8+ |
| Day 0 | Monocyte fraction | % Monocyte | Flow | % of |
| | | | Cytometry | CD45+CD66+ |
| Day 6 | DC | % DC | Flow | ≥60% DC Purity |
| | | Cell count | Cytometry | Cells/mL |
| | | viability | CBC | |
| Day 6 | Cryopreserved | Cell count | CBS | Cells/mL |
| | Lymphocyte | viability | Trypan Blue | |
| Day 18 | T Cell | Viable T Cell | Flow | cells/mL |
| | Concentration | Concentration | Cytometry | |
| Day 18 | Total T Cell Count | Total T Cell | Calculation; | cells |
| | | | Based on T | |
| | | | Cell | |
| | | | Concentration | |
| | | | and Total | |
| | | | Volume | |
| Day 18 | T Cell Culture | Multiples of T | Calculation; | multiples |
| | Expanding | Cell | From | |
| | | Proliferation | Lymphocytes | |
| | | | Seeded | |
| Day 18 | Viability | % Viability | Trypan Blue | percentage |
| | | | Exclusion | |
| Day 18 | Number of Final | # of Vials | Calculation; | number of vials |
| | Formulation Vials | | Based on | |
| | | | Total T cell | |
| | | | Count | |

Binding Properties of Compounds to GITR/GITRL

In certain embodiments, the invention is directed to a compound that binds to a Glucocorticoid-induced receptor ligand (GITRL) that binds at the interface of oligomers. The amino acid residues that are located at the interface of GITRL-oligomers have been described by Zhou et al (PNAS, 2008) with an affinity of 1000 nM or greater, preferably 100 nM or greater, and more preferably of 10 nM or greater.

In certain embodiments, the invention is directed to one of the aforementioned compounds, or a compound different from the aforementioned compounds, that exhibits an affinity for wild type GITRL that is at least about 10-fold greater than the affinity the compound exhibits for human GITRL binds to more than one of an amino acid selected from the group consisting of L42, L44, M71, 172, Q73, T74, K80, I81, Q82, N83, G86, T87, Y88, G114, I116, L118, N120, P121, Q122, F123, I124 and S125 of wild type GITRL sequence as below.

(SEQ ID NO: 3)
MTLHPSPITCEFLFSTALISPKMCLSHLENMPLSHSRTQGAQRSSWKLWL
FCSIVMLLFLCSFSWLIFIFLQLETAKEPCMAKFGPLPSKWQMASSEPPC
VNKVSDWKLEILQNGLYLIYGQVAPNANYNDVAPFEVRLYKNKDMIQTLT
NKSKIQNVGGTYELHVGDTIDLIFNSEHQVLKNNTYWGIILLANPQFIS

Compounds of the invention bind to GITRL. Preferably, compounds bind to GITRL with an affinity (e.g., K_{d}) of 10 μM or less. Without limiting the present disclosure, binding activity may be determined by binding of compounds to cells that express GITRL on their cell surface or a binding of compounds to purified or partially purified GITRL. Binding may be determined using, as non-limiting examples, native or recombinant GITRL, or fragments thereof. Binding of compounds may be determined using methods that are well known to those skilled in the art. Preferred methods for determining binding activity of compounds to GITRL are surface plasmon resonance, isothermal titration calorimetry, ELISA or microscale thermophoresis.

In preferred embodiments, a compound exhibits at least about 10-fold greater binding to wild type GITRL or fragment thereof than the binding the compound exhibits for a mutant of GITRL or mutant fragment thereof. More preferred are compounds that exhibit about 100-fold greater binding to GITRL or fragment thereof, compared to the binding the compound exhibits for a mutant of GITRL or mutant fragment thereof. Most preferred are compounds that exhibit about 1000-fold greater binding to GITRL or fragment thereof, compared to the binding the compound exhibits for a mutant of GITRL or mutant fragment thereof.

Further preferred are compounds exhibiting the aforementioned greater binding to wild type GITRL or fragment thereof compared to a corresponding mutant GITRL or fragment thereof, wherein said mutant bears a substitution in an amino acid selected from the group consisting of L42, L44, M71, I72, Q73, T74, K80, I81, Q82, N83, G86, T87, Y88, G114, I116, L118, N120, P121, Q122, F123, I124 and S125. Further preferred are mutants bearing a substitution at L114 to S125.

Small Molecule Modifiers of GITR/GITRL

Compounds of Formula (I)

In various embodiments, the present invention provides a compound of Formula (I):

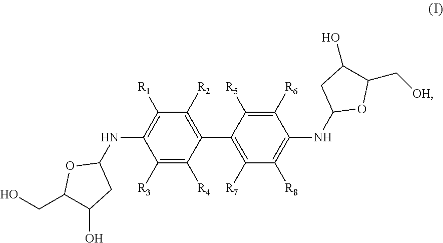

or a pharmaceutically acceptable salt, ester or prodrug thereof;
wherein:

R₁ is hydrogen or an optionally substituted substituent;

R₂ is hydrogen or an optionally substituted substituent;

R₃ is hydrogen or an optionally substituted substituent;

R₄ is hydrogen or an optionally substituted substituent;

R₅ is hydrogen or an optionally substituted substituent;

R₆ is hydrogen or an optionally substituted substituent;

R₇ is hydrogen or an optionally substituted substituent; and

R₈ is hydrogen or an optionally substituted substituent;

wherein optionally any two or more of R₁, R₂, R₃, R₄, R₅, R₆, R₇, or R₈ may be joined together to form one or more rings. In some embodiments, the optionally substituted substituent can be independently selected from halogen (e.g., F, Cl), —OH, —CN, C₁₋₄ alkyl, C₂₋₄ alkenyl, C₂₋₄ alkynyl, C₁₋₄ alkoxy, C₃₋₆ cycloalkyl, C₃₋₆ cycloalkoxy, phenyl, 5 or 6 membered heteroaryl containing 1, 2 or 3 ring heteroatoms independently selected from O, S, and N, 4-7 membered heterocyclyl containing 1 or 2 ring heteroatoms independently selected from O, S, and N, wherein each of the alkyl, alkenyl, alkynyl, alkoxy, cycloalkyl, cycloalkoxy, phenyl, heteroaryl, and heterocyclyl, is optionally substituted with one or more, for example, 1, 2, or 3, substituents independently selected from F, —OH, oxo (as applicable), C₁₋₄ alkyl, fluoro-substituted C₁₋₄ alkyl, C₁₋₄ alkoxy and fluoro-substituted C₁₋₄ alkoxy.

In some embodiments, the present invention provides a compound of Formula I-B, or a pharmaceutically acceptable salt, ester or prodrug thereof,

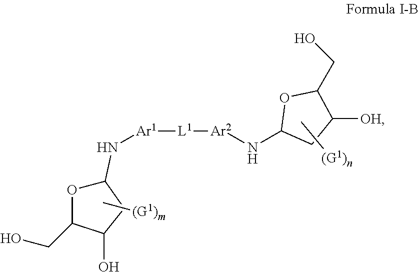

wherein:
Ar¹ and Ar² are each independently an optionally substituted aryl (e.g., phenyl) or an optionally substituted heteroaryl (e.g., 5 or 6 membered heteroaryl, having 1-4 ring heteroatoms independently selected from O, S, and N),
L¹ is a bond, an optionally substituted C₁₋₆ alkylene linker, —O—, —NH—, a protected —NH—, or an optionally substituted C₁₋₆ heteroalkylene linker,
G¹ at each occurrence is independently selected from —OH, halogen (e.g., F), C₁₋₄ alkyl, C₂₋₄ alkenyl, C₂₋₄ alkynyl, C₁₋₄ alkoxy, C₃₋₆ cycloalkoxy, wherein each of the alkyl, alkenyl, alkynyl, alkoxy and cycloalkoxy is optionally substituted with 1-3 substituents independently selected from —OH, C₁₋₄ alkyl, and —F,
m and n are each independently an integer of 0-3 (e.g., 0, 1, or 2).

Typically, L¹ in Formula I-B is a bond. When L¹ is a bond, Ar¹ and Ar² can be connected through any two available positions. In some embodiments, both Ar¹ and Ar² are 6-membered aromatic rings and preferably, the connecting does not result in Ar² being ortho to the —NH— group attached to Ar¹ and/or Ar¹ being ortho to the —NH— group attached to Ar² in Formula I-B. In some embodiments, both Ar¹ and Ar² are 6-membered aromatic rings and preferably, the connecting results in Ar² being para to the —NH— group attached to Ar¹ and/or Ar¹ being para to the —NH— group attached to Ar² in Formula I-B.

In some embodiments, L¹ in Formula I-B is not a bond. For example, in some embodiments, L¹ in Formula I-B can bean unsubstituted straight-chained C₁₋₆alkylene linker, such as a —CH₂—, —CH₂CH₂—, etc. In some embodiments, L¹ in Formula I-B can be an unsubstituted branched C₂₋₆ alkylene linker. As used herein, unsubstituted branched C₂ alkylene should be understood as —CH(CH₃)—. In some embodiments, L¹ in Formula I-B is —O—. In some embodiments, L¹ in Formula I-B is —NH— or a protected —NH—. In some embodiments, L¹ in Formula I-B can be an unsubstituted C₁₋₆ heteroalkylene linker containing 1 or 2 heteroatoms, which can be an oxygen or a nitrogen atom. For example, in some embodiments, L¹ in Formula I-B can be —O—CH₂—, —O—(CH₂)₂—, —O—(CH₂)₂—O—, —NH—(CH₂)₂—O—, etc. In some embodiments, both Ar¹ and Ar² are 6-membered aromatic rings, and L¹ can be para to the —NH— group attached to Ar¹ and/or para to the —NH— group attached to Ar² in Formula I-B.

In some embodiments, Ar¹ and Ar² can both be an optionally substituted phenyl. In some embodiments, Ar¹ and Ar² can both be an optionally substituted heteroaryl, such as a 5-membered heteroaryl having one heteroatom such as thiophenyl or furanyl, a 6-membered heteroaryl having 1 or 2 nitrogen atoms such as pyridyl, pyrimidyl, pyridazinyl, pyrazinyl, or a 5-membered heteroaryl having two or three heteroatoms such as oxazolyl, oxadiazolyl, thiazolyl, thiadiazolyl, triazolyl, isooxazolyl, isothiazolyl, etc. In some embodiments, one of Ar¹ and Ar² is an optionally substituted phenyl and the other of Ar¹ and Ar² is an optionally substituted heteroaryl, such as a 5-membered heteroaryl having one heteroatom such as thiophenyl or furanyl, a 6-membered heteroaryl having 1 or 2 nitrogen atoms such as pyridyl, pyrimidyl, pyridazinyl, pyrazinyl, or a 5-membered heteroaryl having two or three heteroatoms such as oxazolyl, oxadiazolyl, thiazolyl, thiadiazolyl, triazolyl, isooxazolyl, isothiazolyl, etc.

In some embodiments, the “optionally substituted” aryl or heteroaryl groups herein, such as the optionally substituted phenyl, can be unsubstituted or substituted with one or more, for example, 1, 2, or 3, substituents independently selected from halogen (e.g., F, Cl), —OH, —CN, C₁₋₄ alkyl, C₂₋₄ alkenyl, C₂₋₄ alkynyl, C₁₋₄ alkoxy, C₃₋₆ cycloalkyl, C₃₋₆ cycloalkoxy, phenyl, 5 or 6 membered heteroaryl containing 1, 2 or 3 ring heteroatoms independently selected from 0, S, and N, 4-7 membered heterocyclyl containing 1 or 2 ring heteroatoms independently selected from 0, S, and N, wherein each of the alkyl, alkenyl, alkynyl, alkoxy, cycloalkyl, cycloalkoxy, phenyl, heteroaryl, and heterocyclyl, is optionally substituted with one or more, for example, 1, 2, or 3, substituents independently selected from F, —OH, oxo (as applicable), C₁₋₄ alkyl, fluoro-substituted C₁₋₄ alkyl, C₁₋₄ alkoxy and fluoro-substituted C₁₋₄ alkoxy.

As used herein, unless expressly stated to the contrary, combinations of substituents and/or variables are allowable only if such combinations are chemically allowed and result in a stable compound. A “stable” compound is a compound that can be prepared and isolated and whose structure and properties remain or can be caused to remain essentially unchanged for a period of time sufficient to allow use of the compound for the purposes described herein (e.g., therapeutic administration to a subject).

In some embodiments, m is 0. In some embodiments, n is 0. In some embodiments, m and n are both 0.

In some embodiments, at least one of m and n is not 0. In some embodiments, G¹ at each occurrence is independently selected from —OH, F, methyl, ethyl, CF₃, cyclopropyl, cyclobutyl, methoxy, or ethoxy. In some embodiments, m and n are both 1, and the two G¹ groups can be the same or different. When present, G¹ can be attached to any of the four ring carbons of the tetrahydrofuran ring.

In some embodiments, L¹ in Formula I-B is a bond, and the compound of Formula I-B can be characterized as having Formula I-B-1:

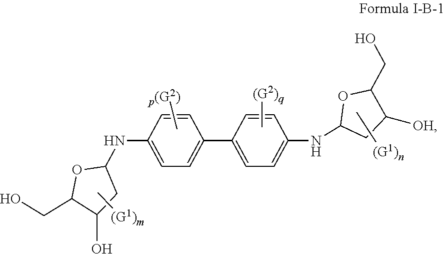

wherein:
G² at each occurrence is independently selected from —OH, halogen (e.g., F), CN, C₁₋₄ alkyl, C₂₋₄ alkenyl, C₂₋₄ alkynyl, C₁₋₄ alkoxy, C₃₋₆ cycloalkoxy, wherein each of the alkyl, alkenyl, alkynyl, alkoxy and cycloalkoxy is optionally substituted with 1-3 substituents independently selected from —OH, C₁₋₄ alkyl, and —F,
p and q are each independently an integer of 0-4 (e.g., 0, 1, or 2); and
G¹, m, and n are defined herein.
In some embodiments, p is 0. In some embodiments, q is 0. In some embodiments, at least one of p and q is not 0. In some embodiments, both p and q are 0. In some embodiments, G² at each occurrence can be independently —OH, F, Cl, Br, I, CN, C₁₋₄ alkyl (e.g., methyl, ethyl, propyl, isopropyl, etc.) optionally substituted with 1-3 fluorine, cyclopropyl, cyclobutyl, C₁₋₄ alkoxy (e.g., methoxy, ethoxy, etc.) optionally substituted with 1-3 fluorine, cyclopropoxy, or cyclobutoxy. In some embodiments, m is 0. In some embodiments, n is 0. In some embodiments, m and n are both 0. In some embodiments, at least one of m and n is not 0. In some embodiments, G¹ at each occurrence is independently selected from —OH, F, methyl, ethyl, CF₃, cyclopropyl, cyclobutyl, methoxy, or ethoxy. In some embodiments, m and n are both 1, and the two G¹ groups can be the same or different.

In some embodiments, m and n are both 0, and the compound of Formula I-B can be characterized as having Formula I-B-2:

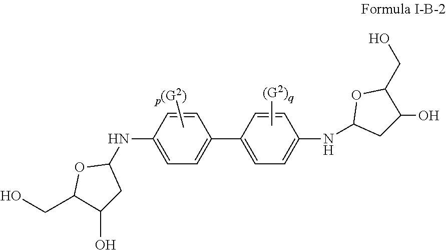

wherein G², p and q are defined herein. In some embodiments, p is 0. In some embodiments, both p and q are 0. In some embodiments, q is 0. In some embodiments, at least one of p and q is not 0. In some embodiments, p and q are the same. In some embodiments, p and q are different. In some embodiments, p can be 0, 1, 2, or 3. In some embodiments, q can be 0, 1, 2, or 3. In some embodiments, G² at each occurrence can be independently —OH, F, Cl, Br, I, CN, C₁₋₄ alkyl (e.g., methyl, ethyl, propyl, isopropyl, etc.) optionally substituted with 1-3 fluorine, cyclopropyl, cyclobutyl, C₁₋₄ alkoxy (e.g., methoxy, ethoxy, etc.) optionally substituted with 1-3 fluorine, cyclopropoxy, or cyclobutoxy.

Compounds described herein can comprise one or more asymmetric centers, and thus can exist in various isomeric forms, e.g., enantiomers and/or diastereomers. For example, the compounds described herein can be in the form of an individual enantiomer, diastereomer or geometric isomer, or can be in the form of a mixture of stereoisomers, including racemic mixtures and mixtures enriched in one or more stereoisomer. Isomers can be isolated from mixtures by methods known to those skilled in the art, including chiral high performance liquid chromatography (HPLC) and the formation and crystallization of chiral salts; or preferred isomers can be prepared by asymmetric syntheses. See, for example, Jacques et al., Enantiomers, Racemates and Resolutions (Wiley Interscience, New York, 1981); Wilen et al., Tetrahedron 33:2725 (1977); Eliel, Stereochemistry of Carbon Compounds (McGraw-Hill, N Y, 1962); and Wilen, Tables of Resolving Agents and Optical Resolutions p. 268 (E. L. Eliel, Ed., Univ. of Notre Dame Press, Notre Dame, Ind. 1972). The disclosure additionally encompasses compounds described herein as individual isomers substantially free of other isomers, and alternatively, as mixtures of various isomers including racemic mixtures.

In some embodiments, the compound of Formula (I) is:

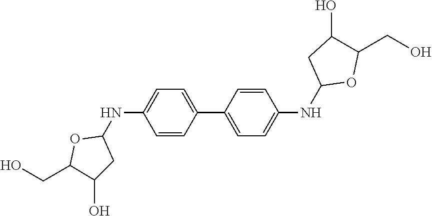 [SMILES: OCC1OC(Nc2ccc(-c3ccc(NC4CC(O)C(CO)O4)cc3)cc2)CC1O]

Compounds of Formula (II)

In various embodiments, the present invention provides a compound of Formula (II):

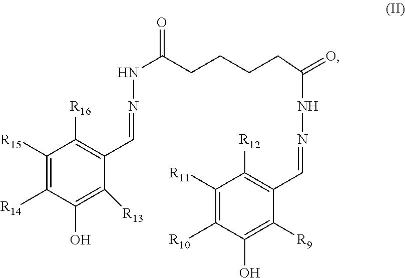

or a pharmaceutically acceptable salt, ester or prodrug thereof;
wherein:

R₉ is hydrogen or an optionally substituted substituent;

R₁₀ is hydrogen or an optionally substituted substituent;

R₁₁ is hydrogen or an optionally substituted substituent;

R₁₂ is hydrogen or an optionally substituted substituent;

R₁₃ is hydrogen or an optionally substituted substituent;

R₁₄ is hydrogen or an optionally substituted substituent;

R₁₅ is hydrogen or an optionally substituted substituent; and

R₁₆ is hydrogen or an optionally substituted substituent;

wherein optionally any two or more of R₉, R₁₀, R₁₁, R₁₂, R₁₃, R₁₄, R₁₅, or R₁₆ may be joined together to form one or more rings. In some embodiments, the optionally substituted substituent can be independently selected from halogen (e.g., F, Cl), —OH, —CN, C₁₋₄ alkyl, C₂₋₄ alkenyl, C₂₋₄ alkynyl, C₁₋₄ alkoxy, C₃₋₆ cycloalkyl, C₃₋₆ cycloalkoxy, phenyl, 5 or 6 membered heteroaryl containing 1, 2 or 3 ring heteroatoms independently selected from O, S, and N, 4-7 membered heterocyclyl containing 1 or 2 ring heteroatoms independently selected from O, S, and N, wherein each of the alkyl, alkenyl, alkynyl, alkoxy, cycloalkyl, cycloalkoxy, phenyl, heteroaryl, and heterocyclyl, is optionally substituted with one or more, for example, 1, 2, or 3, substituents independently selected from F, —OH, oxo (as applicable), C₁₋₄ alkyl, fluoro-substituted C₁₋₄ alkyl, C₁₋₄ alkoxy and fluoro-substituted C₁₋₄ alkoxy.

In some embodiments, the present invention provides a compound of Formula II-B, or a pharmaceutically acceptable salt, ester or prodrug thereof,

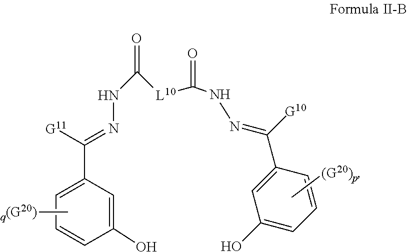

wherein:
L¹⁰ is an optionally substituted C₁₋₁₀ alkylene linker, an optionally substituted C₃₋₁₀ cycloalkylene linker, an optionally substituted phenylene, an optionally substituted heteroarylene, an optionally substituted C₁₋₁₀ heteroalkylene linker, or an optionally substituted heterocyclylene,
G¹⁰ and G¹¹ are independently hydrogen or an optionally substituted C₁₋₄ alkyl,
p and q are independently an integer of 0-4 (e.g., 0, 1, or 2),
G²⁰ at each occurrence is independently selected from halogen (e.g., F, Cl), —OH, —CN, C₁₋₄ alkyl, C₂₋₄ alkenyl, C₂₋₄ alkynyl, C₁₋₄ alkoxy, C₃₋₆ cycloalkyl, C₃₋₆ cycloalkoxy, phenyl, 5 or 6 membered heteroaryl containing 1, 2 or 3 ring heteroatoms independently selected from O, S, and N, 4-7 membered heterocyclyl containing 1 or 2 ring heteroatoms independently selected from 0, S, and N, wherein each of the alkyl, alkenyl, alkynyl, alkoxy, cycloalkyl, cycloalkoxy, phenyl, heteroaryl, and heterocyclyl, is optionally substituted with one or more, for example, 1, 2, or 3, substituents independently selected from F, —OH, oxo (as applicable), C₁₋₄ alkyl, fluoro-substituted C₁₋₄ alkyl, C₁₋₄ alkoxy and fluoro-substituted C₁₋₄ alkoxy.

In some embodiments, L¹⁰ in Formula II-B is an unsubstituted C₁₋₁₀ alkylene linker, such as an unsubstituted straight-chain C₁₋₁₀ alkylene (e.g., C₃₋₆ alkylene) linker or an unsubstituted branched C₁₋₁₀ alkylene linker.

In some embodiments, both G¹⁰ and G¹¹ are hydrogen. In some embodiments, G¹⁰ and G¹¹ are independently hydrogen or C₁₋₄ alkyl (e.g., methyl, ethyl, n-propyl, isopropyl, etc.).

In some embodiments, p is 0. In some embodiments, both p and q are 0. In some embodiments, q is 0. In some embodiments, at least one of p and q is not 0. In some embodiments, p and q are the same. In some embodiments, p and q are different. In some embodiments, p can be 0, 1, 2, or 3. In some embodiments, q can be 0, 1, 2, or 3. In some embodiments, G²⁰ at each occurrence can be independently —OH, F, Cl, Br, I, CN, C₁₋₄ alkyl (e.g., methyl, ethyl, propyl, isopropyl, etc.) optionally substituted with 1-3 fluorine, cyclopropyl, cyclobutyl, C₁₋₄ alkoxy (e.g., methoxy, ethoxy, etc.) optionally substituted with 1-3 fluorine, cyclopropoxy, or cyclobutoxy.

In some embodiments, the compound of Formula (II) is:

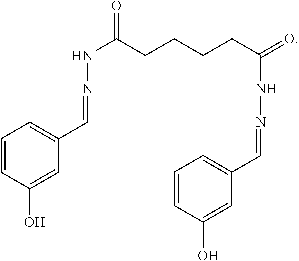 [SMILES: O=C(CCCCC(=O)NN=Cc1cccc(O)c1)NN=Cc1cccc(O)c1]

Non-limiting embodiments of compounds of the invention are provided in Table 1 herein.

TABLE 1
GITR and GITRL Receptor Complex Modifiers

| Compound ID | Compound |
|---|---|
| RMGL171102 (aka 11702) | 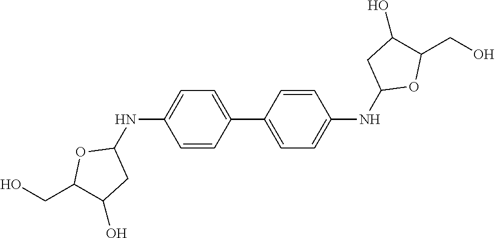 [SMILES: OCC1OC(Nc2ccc(-c3ccc(NC4CC(O)C(CO)O4)cc3)cc2)CC1O] |
| RMGL171103 (aka 11703) | 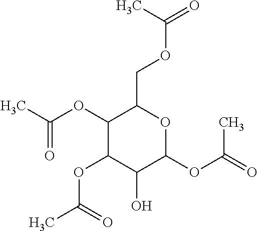 [SMILES: CC(=O)OCC1OC(OC(C)=O)C(O)C(OC(C)=O)C1OC(C)=O] |
| RMGL171104 (aka 11704) | 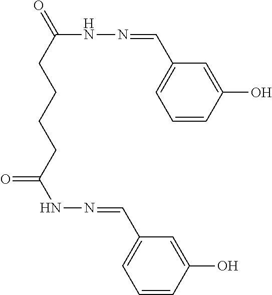 [SMILES: O=C(CCCCC(=O)NN=Cc1cccc(O)c1)NN=Cc1cccc(O)c1] |

As used herein, the term “alkyl” means a straight or branched, saturated aliphatic radical having a chain of carbon atoms. Cₓ alkyl and Cₓ-C_{y}alkyl are typically used where X and Y indicate the number of carbon atoms in the chain. For example, C₁-C₆alkyl includes alkyls that have a chain of between 1 and 6 carbons (e.g., methyl, ethyl, propyl, isopropyl, butyl, sec-butyl, isobutyl, tert-butyl, pentyl, neopentyl, hexyl, and the like). Alkyl represented along with another radical (e.g., as in arylalkyl) means a straight or branched, saturated alkyl divalent radical having the number of atoms indicated or when no atoms are indicated means a bond, e.g., (C₆-C₁₀)aryl(C₀-C₃)alkyl includes phenyl, benzyl, phenethyl, 1-phenylethyl 3-phenylpropyl, and the like. Backbone of the alkyl can be optionally inserted with one or more heteroatoms, such as N, O, or S.

In preferred embodiments, a straight chain or branched chain alkyl has 30 or fewer carbon atoms in its backbone (e.g., C₁-C₃₀ for straight chains, C₃-C₃₀ for branched chains), and more preferably 20 or fewer. Likewise, preferred cycloalkyls have from 3-10 carbon atoms in their ring structure, and more preferably have 5, 6 or 7 carbons in the ring structure. The term “alkyl” (or “lower alkyl”) as used throughout the specification, examples, and claims is intended to include both “unsubstituted alkyls” and “substituted alkyls”, the latter of which refers to alkyl moieties having one or more substituents replacing a hydrogen on one or more carbons of the hydrocarbon backbone.

Unless the number of carbons is otherwise specified, “lower alkyl” as used herein means an alkyl group, as defined above, but having from one to ten carbons, more preferably from one to six carbon atoms in its backbone structure. Likewise, “lower alkenyl” and “lower alkynyl” have similar chain lengths. Throughout the application, preferred alkyl groups are lower alkyls. In preferred embodiments, a substituent designated herein as alkyl is a lower alkyl.

Non-limiting examples of substituents of a substituted alkyl can include halogen, hydroxy, nitro, thiols, amino, azido, imino, amido, phosphoryl (including phosphonate and phosphinate), sulfonyl (including sulfate, sulfonamido, sulfamoyl and sulfonate), and silyl groups, as well as ethers, alkylthios, carbonyls (including ketones, aldehydes, carboxylates, and esters), —CF₃, —CN and the like.

As used herein, the term “alkenyl” refers to unsaturated straight-chain, branched-chain or cyclic hydrocarbon radicals having at least one carbon-carbon double bond. Cₓ alkenyl and Cₓ-C_{y}alkenyl are typically used where X and Y indicate the number of carbon atoms in the chain. For example, C₂-C₆alkenyl includes alkenyls that have a chain of between 2 and 6 carbons and at least one double bond, e.g., vinyl, allyl, propenyl, isopropenyl, 1-butenyl, 2-butenyl, 3-butenyl, 2-methylallyl, 1-hexenyl, 2-hexenyl, 3-hexenyl, and the like). Alkenyl represented along with another radical (e.g., as in arylalkenyl) means a straight or branched, alkenyl divalent radical having the number of atoms indicated. Backbone of the alkenyl can be optionally inserted with one or more heteroatoms, such as N, O, or S.

As used herein, the term “alkynyl” refers to unsaturated hydrocarbon radicals having at least one carbon-carbon triple bond. Cₓ alkynyl and Cₓ-C_{y}alkynyl are typically used where X and Y indicate the number of carbon atoms in the chain. For example, C₂-C₆alkynyl includes alkynls that have a chain of between 2 and 6 carbons and at least one triple bond, e.g., ethynyl, 1-propynyl, 2-propynyl, 1-butynyl, isopentynyl, 1,3-hexa-diyn-yl, n-hexynyl, 3-pentynyl, 1-hexen-3-ynyl and the like. Alkynyl represented along with another radical (e.g., as in arylalkynyl) means a straight or branched, alkynyl divalent radical having the number of atoms indicated. Backbone of the alkynyl can be optionally inserted with one or more heteroatoms, such as N, O, or S.

The terms “alkylene,” “alkenylene,” and “alkynylene” refer to divalent alkyl, alkenyl, and alkynyl” radicals. Prefixes Cₓ and Cₓ-C_{y} are typically used where X and Y indicate the number of carbon atoms in the chain. For example, C₁-C₆alkylene includes methylene, (—CH₂—), ethylene (—CH₂CH₂—), trimethylene (—CH₂CH₂CH₂—), tetramethylene (—CH₂CH₂CH₂CH₂—), 2-methyltetramethylene (—CH₂CH(CH₃)CH₂CH₂—), pentamethylene (—CH₂CH₂CH₂CH₂CH₂—) and the like).

As used herein, the term “alkylidene” means a straight or branched unsaturated, aliphatic, divalent radical having a general formula ═CRₐR_{b}. Non-limiting examples of Ra and Rb are each independently hydrogen, alkyl, substituted alkyl, alkenyl, or substituted alkenyl. Cₓ alkylidene and Cₓ-C_{y}alkylidene are typically used where X and Y indicate the number of carbon atoms in the chain. For example, C₂-C₆alkylidene includes methylidene (═CH₂), ethylidene (═CHCH₃), isopropylidene (═C(CH₃)₂), propylidene (═CHCH₂CH₃), allylidene (═CH—CH═CH₂), and the like).

The term “heteroalkyl”, as used herein, refers to straight or branched chain, or cyclic carbon-containing radicals, or combinations thereof, containing at least one heteroatom. Suitable heteroatoms include, but are not limited to, O, N, Si, P, Se, B, and S, wherein the phosphorous and sulfur atoms are optionally oxidized, and the nitrogen heteroatom is optionally quaternized. Heteroalkyls can be substituted as defined above for alkyl groups.

As used herein, the term “halogen” or “halo” refers to an atom selected from fluorine, chlorine, bromine and iodine. The term “halogen radioisotope” or “halo isotope” refers to a radionuclide of an atom selected from fluorine, chlorine, bromine and iodine.

A “halogen-substituted moiety” or “halo-substituted moiety”, as an isolated group or part of a larger group, means an aliphatic, alicyclic, or aromatic moiety, as described herein, substituted by one or more “halo” atoms, as such terms are defined in this application. For example, halo-substituted alkyl includes haloalkyl, dihaloalkyl, trihaloalkyl, perhaloalkyl and the like (e.g. halosubstituted (C₁-C₃)alkyl includes chloromethyl, dichloromethyl, difluoromethyl, trifluoromethyl (—CF₃), 2,2,2-trifluoroethyl, perfluoroethyl, 2,2,2-trifluoro-1,1-dichloroethyl, and the like).

The term “aryl” refers to monocyclic, bicyclic, or tricyclic fused aromatic ring system. Cₓ aryl and Cₓ-C_{y}aryl are typically used where X and Y indicate the number of carbon atoms in the ring system. For example, C₆-C₁₂ aryl includes aryls that have 6 to 12 carbon atoms in the ring system. Exemplary aryl groups include, but are not limited to, pyridinyl, pyrimidinyl, furanyl, thienyl, imidazolyl, thiazolyl, pyrazolyl, pyridazinyl, pyrazinyl, triazinyl, tetrazolyl, indolyl, benzyl, phenyl, naphthyl, anthracenyl, azulenyl, fluorenyl, indanyl, indenyl, naphthyl, phenyl, tetrahydronaphthyl, benzimidazolyl, benzofuranyl, benzothiofuranyl, benzothiophenyl, benzoxazolyl, benzoxazolinyl, benzthiazolyl, benztriazolyl, benztetrazolyl, benzisoxazolyl, benzisothiazolyl, benzimidazolinyl, carbazolyl, 4aH carbazolyl, carbolinyl, chromanyl, chromenyl, cinnolinyl, decahydroquinolinyl, 2H,6H-1,5,2-dithiazinyl, dihydrofuro[2,3 b]tetrahydrofuran, furanyl, furazanyl, imidazolidinyl, imidazolinyl, imidazolyl, 1H-indazolyl, indolenyl, indolinyl, indolizinyl, indolyl, 3H-indolyl, isatinoyl, isobenzofuranyl, isochromanyl, isoindazolyl, isoindolinyl, isoindolyl, isoquinolinyl, isothiazolyl, isoxazolyl, methylenedioxyphenyl, morpholinyl, naphthyridinyl, octahydroisoquinolinyl, oxadiazolyl, 1,2,3-oxadiazolyl, 1,2,4-oxadiazolyl, 1,2,5-oxadiazolyl, 1,3,4-oxadiazolyl, oxazolidinyl, oxazolyl, oxindolyl, pyrimidinyl, phenanthridinyl, phenanthrolinyl, phenazinyl, phenothiazinyl, phenoxathinyl, phenoxazinyl, phthalazinyl, piperazinyl, piperidinyl, piperidonyl, 4-piperidonyl, piperonyl, pteridinyl, purinyl, pyranyl, pyrazinyl, pyrazolidinyl, pyrazolinyl, pyrazolyl, pyridazinyl, pyridooxazole, pyridoimidazole, pyridothiazole, pyridinyl, pyridyl, pyrimidinyl, pyrrolidinyl, pyrrolinyl, 2H-pyrrolyl, pyrrolyl, quinazolinyl, quinolinyl, 4H-quinolizinyl, quinoxalinyl, quinuclidinyl, tetrahydrofuranyl, tetrahydroisoquinolinyl, tetrahydroquinolinyl, tetrazolyl, 6H-1,2,5-thiadiazinyl, 1,2,3-thiadiazolyl, 1,2,4-thiadiazolyl, 1,2,5-thiadiazolyl, 1,3,4-thiadiazolyl, thianthrenyl, thiazolyl, thienyl, thienothiazolyl, thienooxazolyl, thienoimidazolyl, thiophenyl and xanthenyl, and the like. In some embodiments, 1, 2, 3, or 4 hydrogen atoms of each ring can be substituted by a substituent.

The term “heteroaryl” refers to an aromatic 5-8 membered monocyclic, 8-12 membered fused bicyclic, or 11-14 membered fused tricyclic ring system having 1-3 heteroatoms if monocyclic, 1-6 heteroatoms if bicyclic, or 1-9 heteroatoms if tricyclic, said heteroatoms selected from O, N, or S (e.g., carbon atoms and 1-3, 1-6, or 1-9 heteroatoms of N, O, or S if monocyclic, bicyclic, or tricyclic, respectively. Cₓ heteroaryl and Cₓ-C_{y}heteroaryl are typically used where X and Y indicate the number of carbon atoms in the ring system. For example, C₄-C₉ heteroaryl includes heteroaryls that have 4 to 9 carbon atoms in the ring system. Heteroaryls include, but are not limited to, those derived from benzo[b]furan, benzo[b] thiophene, benzimidazole, imidazo[4,5-c]pyridine, quinazoline, thieno[2,3-c]pyridine, thieno[3,2-b]pyridine, thieno[2, 3-b]pyridine, indolizine, imidazo[1,2a]pyridine, quinoline, isoquinoline, phthalazine, quinoxaline, naphthyridine, quinolizine, indole, isoindole, indazole, indoline, benzoxazole, benzopyrazole, benzothiazole, imidazo[1,5-a]pyridine, pyrazolo[1,5-a]pyridine, imidazo[1,2-a]pyrimidine, imidazo[1,2-c]pyrimidine, imidazo[1,5-a]pyrimidine, imidazo[1,5-c]pyrimidine, pyrrolo[2,3-b]pyridine, pyrrolo[2,3cjpyridine, pyrrolo[3,2-c]pyridine, pyrrolo[3,2-b]pyridine, pyrrolo[2,3-d]pyrimidine, pyrrolo[3,2-d]pyrimidine, pyrrolo [2,3-b]pyrazine, pyrazolo[1,5-a]pyridine, pyrrolo[1,2-b]pyridazine, pyrrolo[1,2-c]pyrimidine, pyrrolo[1,2-a]pyrimidine, pyrrolo[1,2-a]pyrazine, triazo[1,5-a]pyridine, pteridine, purine, carbazole, acridine, phenazine, phenothiazene, phenoxazine, 1,2-dihydropyrrolo[3,2,1-hi]indole, indolizine, pyrido[1,2-a]indole, 2(1H)-pyridinone, benzimidazolyl, benzofuranyl, benzothiofuranyl, benzothiophenyl, benzoxazolyl, benzoxazolinyl, benzthiazolyl, benztriazolyl, benztetrazolyl, benzisoxazolyl, benzisothiazolyl, benzimidazolinyl, carbazolyl, 4aH-carbazolyl, carbolinyl, chromanyl, chromenyl, cinnolinyl, decahydroquinolinyl, 2H,6H-1,5,2-dithiazinyl, dihydrofuro[2,3-b]tetrahydrofuran, furanyl, furazanyl, imidazolidinyl, imidazolinyl, imidazolyl, 1H-indazolyl, indolenyl, indolinyl, indolizinyl, indolyl, 3H-indolyl, isatinoyl, isobenzofuranyl, isochromanyl, isoindazolyl, isoindolinyl, isoindolyl, isoquinolinyl, isothiazolyl, isoxazolyl, methylenedioxyphenyl, morpholinyl, naphthyridinyl, octahydroisoquinolinyl, oxadiazolyl, 1,2,3-oxadiazolyl, 1,2,4-oxadiazolyl, 1,2,5-oxadiazolyl, 1,3,4-oxadiazolyl, oxazolidinyl, oxazolyl, oxepanyl, oxetanyl, oxindolyl, pyrimidinyl, phenanthridinyl, phenanthrolinyl, phenazinyl, phenothiazinyl, phenoxathinyl, phenoxazinyl, phthalazinyl, piperazinyl, piperidinyl, piperidonyl, 4-piperidonyl, piperonyl, pteridinyl, purinyl, pyranyl, pyrazinyl, pyrazolidinyl, pyrazolinyl, pyrazolyl, pyridazinyl, pyridooxazole, pyridoimidazole, pyridothiazole, pyridinyl, pyridyl, pyrimidinyl, pyrrolidinyl, pyrrolinyl, 2H-pyrrolyl, pyrrolyl, quinazolinyl, quinolinyl, 4H-quinolizinyl, quinoxalinyl, quinuclidinyl, tetrahydrofuranyl, tetrahydroisoquinolinyl, tetrahydropyranyl, tetrahydroquinolinyl, tetrazolyl, 6H-1,2,5-thiadiazinyl, 1,2,3-thiadiazolyl, 1,2,4-thiadiazolyl, 1,2,5-thiadiazolyl, 1,3,4-thiadiazolyl, thianthrenyl, thiazolyl, thienyl, thienothiazolyl, thienooxazolyl, thienoimidazolyl, thiophenyl and xanthenyl. Some exemplary heteroaryl groups include, but are not limited to, pyridyl, furyl or furanyl, imidazolyl, benzimidazolyl, pyrimidinyl, thiophenyl or thienyl, pyridazinyl, pyrazinyl, quinolinyl, indolyl, thiazolyl, naphthyridinyl, 2-amino-4-oxo-3,4-dihydropteridin-6-yl, tetrahydroisoquinolinyl, and the like. In some embodiments, 1, 2, 3, or 4 hydrogen atoms of each ring may be substituted by a substituent.

The term “cyclyl” or “cycloalkyl” refers to saturated and partially unsaturated cyclic hydrocarbon groups having 3 to 12 carbons, for example, 3 to 8 carbons, and, for example, 3 to 6 carbons. Cₓcyclyl and Cₓ-C_{y}cyclyl are typically used where X and Y indicate the number of carbon atoms in the ring system. For example, C₃-C₈ cyclyl includes cyclyls that have 3 to 8 carbon atoms in the ring system. The cycloalkyl group additionally can be optionally substituted, e.g., with 1, 2, 3, or 4 substituents. C₃-C₁₀cyclyl includes cyclopropyl, cyclobutyl, cyclopentyl, cyclohexyl, cyclohexenyl, 2,5-cyclohexadienyl, cycloheptyl, cyclooctyl, bicyclo[2.2.2]octyl, adamantan-1-yl, decahydronaphthyl, oxocyclohexyl, dioxocyclohexyl, thiocyclohexyl, 2-oxobicyclo [2.2.1]hept-1-yl, and the like.

Aryl and heteroaryls can be optionally substituted with one or more substituents at one or more positions, for example, halogen, alkyl, aralkyl, alkenyl, alkynyl, cycloalkyl, hydroxyl, amino, nitro, sulfhydryl, imino, amido, phosphate, phosphonate, phosphinate, carbonyl, carboxyl, silyl, ether, alkylthio, sulfonyl, ketone, aldehyde, ester, a heterocyclyl, an aromatic or heteroaromatic moiety, —CF₃, —CN, or the like.

The term “heterocyclyl” refers to a nonaromatic 4-8 membered monocyclic, 8-12 membered bicyclic, or 11-14 membered tricyclic ring system having 1-3 heteroatoms if monocyclic, 1-6 heteroatoms if bicyclic, or 1-9 heteroatoms if tricyclic, said heteroatoms selected from O, N, or S (e.g., carbon atoms and 1-3, 1-6, or 1-9 heteroatoms of N, O, or S if monocyclic, bicyclic, or tricyclic, respectively). Cₓheterocyclyl and Cₓ-C_{y}heterocyclyl are typically used where X and Y indicate the number of carbon atoms in the ring system. For example, C₄-C₉ heterocyclyl includes heterocyclyls that have 4-9 carbon atoms in the ring system. In some embodiments, 1, 2 or 3 hydrogen atoms of each ring can be substituted by a substituent. Exemplary heterocyclyl groups include, but are not limited to piperazinyl, pyrrolidinyl, dioxanyl, morpholinyl, tetrahydrofuranyl, piperidyl, 4-morpholyl, 4-piperazinyl, pyrrolidinyl, perhydropyrrolizinyl, 1,4-diazaperhydroepinyl, 1,3-dioxanyl, 1,4-dioxanyl and the like.

The terms “bicyclic” and “tricyclic” refers to fused, bridged, or joined by single bond polycyclic ring assemblies.

The term “cyclylalkylene” means a divalent aryl, heteroaryl, cyclyl, or heterocyclyl.

As used herein, the term “fused ring” refers to a ring that is bonded to another ring to form a compound having a bicyclic structure when the ring atoms that are common to both rings are directly bound to each other. Non-exclusive examples of common fused rings include decalin, naphthalene, anthracene, phenanthrene, indole, furan, benzofuran, quinoline, and the like. Compounds having fused ring systems can be saturated, partially saturated, cyclyl, heterocyclyl, aromatics, heteroaromatics, and the like.

The term “carbocyclyl” as used either alone or in combination with another radical, means a mono- bi- or tricyclic ring structure consisting of 3 to 14 carbon atoms. In some embodiments, one or more of the hydrogen atoms of a carbocyclyl may be optionally substituted by a substituent.

The term “carbocycle” refers to fully saturated ring systems and saturated ring systems and partially saturated ring systems and aromatic ring systems and non-aromatic ring systems and unsaturated ring systems and partially unsaturated ring systems. The term “carbocycle” encompasses monocyclic, bicyclic, polycyclic, spirocyclic, fused, bridged, or linked ring systems. In some embodiments, one or more of the hydrogen atoms of a carbocycle may be optionally substituted by a substituent. In some embodiments the carbocycle optionally comprises one or more heteroatoms. In some embodiments the heteroatoms are selected from N, O, S, or P.

The terms “cyclic”, “cyclic group” and “ring” or “rings” means carbocycles, which can be fully saturated, saturated, partially saturated, unsaturated, partially unsaturated non-aromatic or aromatic that may or may not be substituted and which optionally can comprise one or more heteroatoms. In some embodiments the heteroatoms are selected from N, O, S, or P. In some embodiments, one or more of the hydrogen atoms of a ring may be optionally substituted by a substituent. In some embodiments, the ring or rings may be monocyclic, bicyclic, polycyclic, spirocyclic, fused, bridged, or linked.

The term “spiro-cycloalkyl” (spiro) means spirocyclic rings where the ring is linked to the molecule through a carbon atom, and wherein the resulting carbocycle is formed by alkylene groups. The term “spiro-C₃-C₈-cycloalkyl” (spiro) means 3-8 membered, spirocyclic rings where the ring is linked to the molecule through a carbon atom, and wherein the resulting 3-8 membered carbocycle is formed by alkylene groups with 2 to 7 carbon atoms. The term “spiro-C₅-cycloalkyl” (spiro) means 5 membered, spirocyclic rings where the ring is linked to the molecule through a carbon atom, wherein the resulting 5 membered carbocycle is formed by an alkylene group with 4 carbon atoms.

The term “spiro-cycloalkenyl” (spiro) means spirocyclic rings where the ring is linked to the molecule through a carbon atom, and wherein the resulting carbocycle is formed by alkenylene groups. The term “spiro-C₃-C₈-cycloalkenyl” (spiro) means 3-8 membered, spirocyclic rings where the ring is linked to the molecule through a carbon atom, wherein the resulting 3-8 membered carbocycle is formed by alkenylene groups with 2 to 7 carbon atoms. The term “spiro-C₅-cycloalkenyl” (spiro) means 5 membered, spirocyclic rings where the ring is linked to the molecule through a carbon atom, wherein the resulting 5 membered carbocycle is formed by alkenylene groups with 4 carbon atoms.

The term “spiro-heterocyclyl” (spiro) means saturated or unsaturated spirocyclic rings, which may contain one or more heteroatoms, where the ring may be linked to the molecule through a carbon atom or optionally through a nitrogen atom, if a nitrogen atom is present. In some embodiments, the heteroatom is selected from O, N, S, or P. In some embodiments, the heteroatom is O, S, or N. The term “spiro-C₃-C₈-heterocyclyl” (spiro) means 3-8 membered, saturated or unsaturated, spirocyclic rings which may contain one or more heteroatoms, where the ring may be linked to the molecule through a carbon atom or optionally through a nitrogen atom, if a nitrogen atom is present. In some embodiments, the heteroatom is selected from O, N, S, or P. In some embodiments, the heteroatom is O, S, or N. The term “spiro-C₅-heterocyclyl” (spiro) means 5 membered, saturated or unsaturated, spirocyclic rings which may contain one or more heteroatoms, where the ring may be linked to the molecule through a carbon atom or optionally through a nitrogen atom, if a nitrogen atom is present. In some embodiments, the heteroatom is selected from O, N, S, or P. In some embodiments, the heteroatom is O, S, or N.

In some embodiments, one or more of the hydrogen atoms of a spirocyclic ring may be optionally substituted by a substituent. In some embodiments, one or more hydrogen atoms of a spiro-cycloalkyl may be optionally substituted by a substituent. In some embodiments, one or more hydrogen atoms of a spiro-C₃-C₈-cycloalkyl may be optionally substituted by a substituent. In some embodiments, one or more hydrogen atoms of a spiro-C₅-cycloalkyl may be optionally substituted by a substituent. In some embodiments, one or more hydrogen atoms of a spiro-cycloalkenyl may be optionally substituted by a substituent. In some embodiments, one or more hydrogen atoms of a spiro-C₃-C₈-cycloalkenyl may be optionally substituted by a substituent. In some embodiments, one or more hydrogen atoms of a spiro-C₅-cycloalkenyl may be optionally substituted by a substituent. In some embodiments, one or more hydrogen atoms of a spiro-heterocycyl may be optionally substituted by a substituent. In some embodiments, one or more hydrogen atoms of a spiro-C₃-C₈-heterocycyl may be optionally substituted by a substituent. In some embodiments, one or more hydrogen atoms of a spiro-C₅-heterocycyl may be optionally substituted by a substituent.

As used herein, the term “carbonyl” means the radical —C(O)—. It is noted that the carbonyl radical can be further substituted with a variety of substituents to form different carbonyl groups including acids, acid halides, amides, esters, ketones, and the like.

The term “carboxy” means the radical —C(O)O—. It is noted that compounds described herein containing carboxy moieties can include protected derivatives thereof, i.e., where the oxygen is substituted with a protecting group. Suitable protecting groups for carboxy moieties include benzyl, tert-butyl, and the like. The term “carboxyl” means —COOH.

The term “cyano” means the radical —CN.

The term, “heteroatom” refers to an atom that is not a carbon atom. Particular examples of heteroatoms include, but are not limited to nitrogen, oxygen, sulfur and halogens. A “heteroatom moiety” includes a moiety where the atom by which the moiety is attached is not a carbon. Examples of heteroatom moieties include —N═, —NR^{N}—, —N⁺(O⁻)═, —O—, —S— or —S(O)₂—, —OS(O)₂—, and —SS—, wherein R^{N} is H or a further substituent.

The term “hydroxy” means the radical —OH.

The term “imine derivative” means a derivative comprising the moiety —C(NR)—, wherein R comprises a hydrogen or carbon atom alpha to the nitrogen.

The term “nitro” means the radical —NO₂.

An “oxaaliphatic,” “oxaalicyclic”, or “oxaaromatic” mean an aliphatic, alicyclic, or aromatic, as defined herein, except where one or more oxygen atoms (—O—) are positioned between carbon atoms of the aliphatic, alicyclic, or aromatic respectively.

An “oxoaliphatic,” “oxoalicyclic”, or “oxoaromatic” means an aliphatic, alicyclic, or aromatic, as defined herein, substituted with a carbonyl group. The carbonyl group can be an aldehyde, ketone, ester, amide, acid, or acid halide.

As used herein, the term “oxo” means the substituent ═O.

As used herein, the term, “aromatic” means a moiety wherein the constituent atoms make up an unsaturated ring system, all atoms in the ring system are sp² hybridized and the total number of pi electrons is equal to 4n+2. An aromatic ring can be such that the ring atoms are only carbon atoms (e.g., aryl) or can include carbon and non-carbon atoms (e.g., heteroaryl).

As used herein, the term “substituted” refers to independent replacement of one or more (typically 1, 2, 3, 4, or 5) of the hydrogen atoms on the substituted moiety with substituents independently selected from the group of substituents listed below in the definition for “substituents” or otherwise specified. In general, a non-hydrogen substituent can be any substituent that can be bound to an atom of the given moiety that is specified to be substituted. Examples of substituents include, but are not limited to, acyl, acylamino, acyloxy, aldehyde, alicyclic, aliphatic, alkanesulfonamido, alkanesulfonyl, alkaryl, alkenyl, alkoxy, alkoxycarbonyl, alkyl, alkylamino, alkylcarbanoyl, alkylene, alkylidene, alkylthios, alkynyl, amide, amido, amino, amidine, aminoalkyl, aralkyl, aralkylsulfonamido, arenesulfonamido, arenesulfonyl, aromatic, aryl, arylamino, arylcarbanoyl, aryloxy, azido, carbamoyl, carbonyl, carbonyls including ketones, carboxy, carboxylates, CF₃, cyano (CN), cycloalkyl, cycloalkylene, ester, ether, haloalkyl, halogen, halogen, heteroaryl, heterocyclyl, hydroxy, hydroxyalkyl, imino, iminoketone, ketone, mercapto, nitro, oxaalkyl, oxo, oxoalkyl, phosphoryl (including phosphonate and phosphinate), silyl groups, sulfonamido, sulfonyl (including sulfate, sulfamoyl and sulfonate), thiols, and ureido moieties, each of which may optionally also be substituted or unsubstituted. In some cases, two substituents, together with the carbon(s) to which they are attached to, can form a ring. In some cases, two or more substituents, together with the carbon(s) to which they are attached to, can form one or more rings.

Substituents may be protected as necessary and any of the protecting groups commonly used in the art may be employed. Non-limiting examples of protecting groups may be found, for example, in Greene and Wuts, Protective Groups in Organic Synthesis, 44^{th}. Ed., Wiley & Sons, 2006.

The terms “alkoxyl” or “alkoxy” as used herein refers to an alkyl group, as defined above, having an oxygen radical attached thereto. Representative alkoxyl groups include methoxy, ethoxy, propyloxy, tert-butoxy, n-propyloxy, iso-propyloxy, n-butyloxy, iso-butyloxy, and the like. An “ether” is two hydrocarbons covalently linked by an oxygen. Accordingly, the substituent of an alkyl that renders that alkyl an ether is or resembles an alkoxyl, such as can be represented by one of —O-alkyl, —O-alkenyl, and —O-alkynyl. Aroxy can be represented by —O-aryl or O-heteroaryl, wherein aryl and heteroaryl are as defined below. The alkoxy and aroxy groups can be substituted as described above for alkyl.

The term “aralkyl”, as used herein, refers to an alkyl group substituted with an aryl group (e.g., an aromatic or heteroaromatic group).

The term “alkylthio” refers to an alkyl group, as defined above, having a sulfur radical attached thereto. In preferred embodiments, the “alkylthio” moiety is represented by one of —S— alkyl, —S-alkenyl, and —S-alkynyl. Representative alkylthio groups include methylthio, ethylthio, and the like. The term “alkylthio” also encompasses cycloalkyl groups, alkene and cycloalkene groups, and alkyne groups. “Arylthio” refers to aryl or heteroaryl groups.

The term “sulfinyl” means the radical —SO—. It is noted that the sulfinyl radical can be further substituted with a variety of substituents to form different sulfinyl groups including sulfinic acids, sulfinamides, sulfinyl esters, sulfoxides, and the like.

The term “sulfonyl” means the radical —SO₂—. It is noted that the sulfonyl radical can be further substituted with a variety of substituents to form different sulfonyl groups including sulfonic acids (—SO₃H), sulfonamides, sulfonate esters, sulfones, and the like.

The term “thiocarbonyl” means the radical —C(S)—. It is noted that the thiocarbonyl radical can be further substituted with a variety of substituents to form different thiocarbonyl groups including thioacids, thioamides, thioesters, thioketones, and the like.

As used herein, the term “amino” means —NH₂. The term “alkylamino” means a nitrogen moiety having at least one straight or branched unsaturated aliphatic, cyclyl, or heterocyclyl radicals attached to the nitrogen. For example, representative amino groups include —NH₂, —NHCH₃, —N(CH₃)₂, —NH(C₁-C₁₀alkyl), —N(C₁-C₁₀alkyl)₂, and the like. The term “alkylamino” includes “alkenylamino,” “alkynylamino,” “cyclylamino,” and “heterocyclylamino.” The term “arylamino” means a nitrogen moiety having at least one aryl radical attached to the nitrogen. For example —NHaryl, and —N(aryl)₂. The term “heteroarylamino” means a nitrogen moiety having at least one heteroaryl radical attached to the nitrogen. For example —NHheteroaryl, and —N(heteroaryl)₂. Optionally, two substituents together with the nitrogen can also form a ring. Unless indicated otherwise, the compounds described herein containing amino moieties can include protected derivatives thereof. Suitable protecting groups for amino moieties include acetyl, tertbutoxycarbonyl, benzyloxycarbonyl, and the like.

The term “aminoalkyl” means an alkyl, alkenyl, and alkynyl as defined above, except where one or more substituted or unsubstituted nitrogen atoms (—N—) are positioned between carbon atoms of the alkyl, alkenyl, or alkynyl. For example, an (C₂-C₆) aminoalkyl refers to a chain comprising between 2 and 6 carbons and one or more nitrogen atoms positioned between the carbon atoms.

The term “alkoxyalkoxy” means —O-(alkyl)-O-(alkyl), such as —OCH₂CH₂OCH₃, and the like.

The term “alkoxycarbonyl” means —C(O)O-(alkyl), such as —C(═O)OCH₃, —C(═O)OCH₂CH₃, and the like.

The term “alkoxyalkyl” means -(alkyl)-O-(alkyl), such as —CH₂OCH₃, —CH₂OCH₂CH₃, and the like.

The term “aryloxy” means —O-(aryl), such as —O-phenyl, —O-pyridinyl, and the like.

The term “arylalkyl” means -(alkyl)-(aryl), such as benzyl (i.e., —CH₂phenyl), —CH₂-pyrindinyl, and the like.

The term “arylalkyloxy” means —O-(alkyl)-(aryl), such as —O-benzyl, —O—CH₂-pyridinyl, and the like.

The term “cycloalkyloxy” means —O-(cycloalkyl), such as —O-cyclohexyl, and the like.

The term “cycloalkylalkyloxy” means —O-(alkyl)-(cycloalkyl, such as —OCH₂cyclohexyl, and the like.

The term “aminoalkoxy” means —O-(alkyl)-NH₂, such as —OCH₂NH₂, —OCH₂CH₂NH₂, and the like.

The term “mono- or di-alkylamino” means —NH(alkyl) or —N(alkylxalkyl), respectively, such as —NHCH₃, —N(CH₃)₂, and the like.

The term “mono- or di-alkylaminoalkoxy” means —O-(alkyl)-NH(alkyl) or —O-(alkyl)-N(alkylxalkyl), respectively, such as —OCH₂NHCH₃, —OCH₂CH₂N(CH₃)₂, and the like.

The term “arylamino” means —NH(aryl), such as —NH-phenyl, —NH-pyridinyl, and the like.

The term “arylalkylamino” means —NH-(alkyl)-(aryl), such as —NH-benzyl, —NHCH₂-pyridinyl, and the like.

The term “alkylamino” means —NH(alkyl), such as —NHCH₃, —NHCH₂CH₃, and the like.

The term “cycloalkylamino” means —NH-(cycloalkyl), such as —NH-cyclohexyl, and the like.

The term “cycloalkylalkylamino”-NH-(alkyl)-(cycloalkyl), such as —NHCH₂-cyclohexyl, and the like.

Some commonly used abbreviations are: Me is methyl, Et is ethyl, Ph is phenyl, t-Bu is tert-butyl.

It is noted in regard to all of the definitions provided herein that the definitions should be interpreted as being open ended in the sense that further substituents beyond those specified may be included. Hence, a C₁ alkyl indicates that there is one carbon atom but does not indicate what are the substituents on the carbon atom. Hence, a C₁ alkyl comprises methyl (i.e., —CH₃) as well as —CRₐR_{b}R_{c} where Rₐ, R_{b}, and R_{c} can each independently be hydrogen or any other substituent where the atom alpha to the carbon is a heteroatom or cyano. Hence, CF₃, CH₂OH and CH₂CN are all C₁ alkyls.

Unless otherwise stated, structures depicted herein are meant to include compounds which differ only in the presence of one or more isotopically enriched atoms. For example, compounds having the present structure except for the replacement of a hydrogen atom by a deuterium or tritium, or the replacement of a carbon atom by a ¹³C- or ¹⁴C-enriched carbon are within the scope of the invention.

Synthetic Preparation. In various embodiments, compounds of the present invention as disclosed herein may be synthesized using any synthetic method available to one of skill in the art. In various embodiments, the compounds of the present invention disclosed herein can be prepared in a variety of ways known to one skilled in the art of organic synthesis, and in analogy with the exemplary compounds whose synthesis is described herein. The starting materials used in preparing these compounds may be commercially available or prepared by known methods. Preparation of compounds can involve the protection and de-protection of various chemical groups. The need for protection and de-protection, and the selection of appropriate protecting groups can be readily determined by one skilled in the art. The chemistry of protecting groups can be found, for example, in Greene and Wuts, Protective Groups in Organic Synthesis, 44th. Ed., Wiley & Sons, 2006, which is incorporated herein by reference in its entirety. Non-limiting examples of synthetic methods used to prepare various embodiments of compounds of the present invention are disclosed in the Examples section herein. The reactions of the processes described herein can be carried out in suitable solvents which can be readily selected by one of skill in the art of organic synthesis. Suitable solvents can be substantially nonreactive with the starting materials (reactants), the intermediates, or products at the temperatures at which the reactions are carried out, i.e., temperatures which can range from the solvent's freezing temperature to the solvent's boiling temperature. A given reaction can be carried out in one solvent or a mixture of more than one solvent. Depending on the particular reaction step, suitable solvents for a particular reaction step can be selected.

Use with Polymers. In various embodiments, the compounds of the present invention as disclosed herein may be conjugated to a polymer matrix, e.g., for controlled delivery of the compound. The compound may be conjugated via a covalent bond or non-covalent association. In certain embodiments wherein the compound is covalently linked to the polymer matrix, the linkage may comprise a moiety that is cleavable under biological conditions (e.g., ester, amide, carbonate, carbamate, imide, etc.). In certain embodiments, the conjugated compound may be a pharmaceutically acceptable salt, ester, or prodrug of a compound disclosed herein. A compound as disclosed herein may be associated with any type of polymer matrix known in the art for the delivery of therapeutic agents.

Agonists of GITR/GITRL Receptor Complex

Further provided herein are peptide agonists of GITR. In one embodiment, the peptide agonists of GITR comprises, consists of or consists essentially of a peptide having the sequence set forth in SEQ ID NO:1 or a mutant or functional equivalent thereof.

(SEQ ID NO: 1)
GAMASQLETAKEPCMAKFGPLPSKWQMASSEPPCVNKVSDWKLEILQNGL
YLIYGQVAPNANYNDVAPFEVRLYKNKDMIQTLTNKSKIQNVGGTYELHV
GDTIDLIFNSEHQVLKNNTYWGIILLANPQFIS(GSGSGSGS)KEPCMAK
FGPLPSKWQMASSEPPCVNKVSDWKLEILQNGLYLIYGQVAPNANYNDVA
PFEVRLYKNKDMIQTLTNKSKIQNVGGTYELHVGDTIDLIFNSEHQVLKN
NTYWGIILLANPQFIS(GSGSGSGS)nKEPCMAKFGPLPSKWQMASSEPP
CVNKVSDWKLEILQNGLYLIYGQVAPNANYNDVAPFEVRLYKNKDMIQTL
TNKSKIQNVGGTYELHVGDTIDLIFNSEHQVLKNNTYWGIILLANPQFI
S, wherein n = 1 to 4.

In another embodiment, the peptide agonists of GITR comprises, consists of or consists essentially of a peptide having the sequence KEPCMAKFGPLPSKWQMASSEPPCVNKVSDWKLEILQNGLYLIYGQVAPNANYNDVAP FEVRLYKNKDMIQTLTNKSKIQNVGGTYELHVGDTIDLIFNSEHQVLKNNTYWGIILLAN PQFIS (SEQ ID NO:4). Functional GITR is an oligomer. Peptide comprising, consisting of or consisting essentially of the sequence in SEQ ID NO:4 binds to a monomer of functional GITR oligomer (for example, trimer). In some embodiments, the GITR agonist is an oligomer of SEQ ID NO:4, wherein each monomer comprising the sequence set forth in SEQ ID NO:4 is linked via a linker sequence. In an exemplary embodiment, the linker is GSGSGSGS (SEQ ID NO:5). As set forth herein, SEQ ID NO:1 comprises an oligomer of SEQ ID NO:4, wherein each monomer of SEQ ID NO:4 is linked via the linker having the sequence set forth in SEQ ID NO:5.

In another embodiment, the peptide agonists of GITR comprise, consist of or consist essentially of a peptide having the sequence set forth in SEQ ID NO: 2 or a mutant or functional equivalent thereof.

(SEQ ID NO: 2)
TGGRNSIRYSELAPLFDTTRVYLVDNKSTDVASLNYQNDHSNFLTTVIQN
NDYSPGEASTQTINLDDRSHWGGDLKTILHTNMPNVNEFMFTNKFKARVM
VSRSLTKDKQVELKYEWVEFTLPEGNYSETMTIDLMNNAIVEHYLKVGRQ
NGVLESDIGVKFDTRNFRLGFDPVTGLVMPGVYTNEAFHPDIILLPGCGV
DFTHSRLSNLLGIRKRQPFQEGFRITYDDLEGGNIPALLDVDAYQASLKD
DTEQGGDGAGGGNNSGSGAEENSNAAAAAMQPVEDMNDHAINGSTFATRA
EEKRAEAEAAAEAAAPAAQPEVEKPQKKPVIKPLTEDSKKRSYNLISNDS
TFTQYRSWYLAYNYGDPQTGIRSWTLLCTPDVTCGSEQVYWSLPDMMQDP
VTFRSTSQISNFPVVGAELLPVHSKSFYNDQAVYSQLIRQFTSLTHVFNR
FPENQILARPPAPTITTVSENVPALTDHGTLPLRNSIGGVQRVTITDARR
RTCPYVYKALGIVSPRVLSSRT(GSGSGSGS)ₙGAMASQLETAKEPCMAK
FGPLPSKWQMASSEPPCVNKVSDWKLEILQNGLYLIYGQVAPNANYNDVA
PFEVRLYKNKDMIQTLTNKSKIQNVGGTYELHVGDTIDLIFNSEHQVLKN
NTYWGIILLANPQFISGSHHHHHH

wherein the underlined NGS sequence is a glycosylation site; and n=1 to 4.

Further provided herein are compositions comprising GITR agonists. In one embodiment, the composition comprises the peptide set forth in SEQ ID NO: 1. In another embodiment, the composition comprises the peptide set forth in SEQ ID NO: 2. When administered therapeutically, the peptide agonists of GITR are compositions that comprises peptides having the sequences set forth in SEQ ID NO: 1 or SEQ ID NO: 2 and further comprise a pharmaceutically acceptable solution or carrier.

Additional Non-Limiting Embodiments of the Invention

In some embodiments, peptides comprising, consisting of or consisting essentially of the sequences set forth in SEQ ID NO: 1 or SEQ ID NO: 2, or analogs, pharmaceutical equivalents and/or peptidomimetics thereof are modified peptides. “Modified peptide” may include the incorporation of lactam-bridge, head-to-tail cyclization, non-natural amino acids into the peptides of the invention, including synthetic non-native amino acids, substituted amino acids, or one or more D-amino acids into the peptides (or other components of the composition, with exception for protease recognition sequences) is desirable in certain situations. D-amino acid-containing peptides exhibit increased stability in vitro or in vivo compared to L-amino acid-containing forms. Thus, the construction of peptides incorporating D-amino acids can be particularly useful when greater in vivo or intracellular stability is desired or required. More specifically, D-peptides are resistant to endogenous peptidases and proteases, thereby providing better oral trans-epithelial and transdermal delivery of linked drugs and conjugates, improved bioavailability of membrane-permanent complexes (see below for further discussion), and prolonged intravascular and interstitial lifetimes when such properties are desirable. The use of D-isomer peptides can also enhance transdermal and oral trans-epithelial delivery of linked drugs and other cargo molecules. Additionally, D-peptides cannot be processed efficiently for major histocompatibility complex class II-restricted presentation to T helper cells, and are therefore less likely to induce humoral immune responses in the whole organism. Peptide conjugates can therefore be constructed using, for example, D-isomer forms of cell penetrating peptide sequences, L-isomer forms of cleavage sites, and D-isomer forms of therapeutic peptides. Therefore, in some embodiments the peptides as disclosed comprise L and D amino acids, wherein no more than 1, 2, 3, 4, 5, 6, 7, 8, 9, or 10 D-amino acids are included. In certain aspects, the peptides comprise more than 10 D-amino acids, and in certain aspects all the amino acids of the peptides are D-amino acids.

In some embodiments, peptides comprising, consisting of or consisting essentially of the sequences set forth in SEQ ID NO: 1 or SEQ ID NO: 2 or analogs, pharmaceutical equivalents and/or peptidomimetics thereof are retro-inverso peptides of the said peptides or analogs, pharmaceutical equivalents and/or peptidomimetics thereof. A “retro-inverso peptide” refers to a peptide with a reversal of the direction of the peptide bond on at least one position, i.e., a reversal of the amino- and carboxy-termini with respect to the side chain of the amino acid. Thus, a retro-inverso analogue has reversed termini and reversed direction of peptide bonds while approximately maintaining the topology of the side chains as in the native peptide sequence. The retro-inverso peptide can contain L-amino acids or D-amino acids, or a mixture of L-amino acids and D-amino acids, up to all of the amino acids being the D-isomer. Partial retro-inverso peptide analogues are polypeptides in which only part of the sequence is reversed and replaced with enantiomeric amino acid residues. Since the retro-inverted portion of such an analogue has reversed amino and carboxyl termini, the amino acid residues flanking the retro-inverted portion are replaced by side-chain-analogous α-substituted geminal-diaminomethanes and malonates, respectively. Retro-inverso forms of cell penetrating peptides have been found to work as efficiently in translocating across a membrane as the natural forms. Synthesis of retro-inverso peptide analogues are described in Bonelli, F. et al., Int J Pept Protein Res. 24(6):553-6 (1984); Verdini, A and Viscomi, G. C, J. Chem. Soc. Perkin Trans. 1:697-701 (1985); and U.S. Pat. No. 6,261,569, which are incorporated herein in their entirety by reference. Processes for the solid-phase synthesis of partial retro-inverso peptide analogues have been described (EP 97994-B) which is also incorporated herein in its entirety by reference.

Other variants of the peptides described herein (peptides comprising, consisting of or consisting essentially of the sequences set forth in SEQ ID NO: 1 or SEQ ID NO: 2) can comprise conservatively substituted sequences, meaning that one or more amino acid residues of an original peptide are replaced by different residues, and that the conservatively substituted peptide retains a desired biological activity, i.e., function as an agonist of GITR (for example, SEQ ID NO:1 or SEQ ID NO:2) that is essentially equivalent to that of the original peptide. Examples of conservative substitutions include substitution of amino acids that do not alter the secondary and/or tertiary structure of peptides set forth in SEQ ID NO:1 or SEQ ID NO:2, substitutions that do not change the overall or local hydrophobic character, substitutions that do not change the overall or local charge, substitutions by residues of equivalent side-chain size, or substitutions by side-chains with similar reactive groups.

Other examples involve substitution of amino acids that have not been evolutionarily conserved in the parent sequence across species. Advantageously, in some embodiments, these conserved amino acids and structures are not altered when generating conservatively substituted sequences.

A given amino acid can be replaced by a residue having similar physiochemical characteristics, e.g., substituting one aliphatic residue for another (such as Ile, Val, Leu, or Ala for one another), or substitution of one polar residue for another (such as between Lys and Arg; Glu and Asp; or Gln and Asn). Other such conservative substitutions, e.g., substitutions of entire regions having similar hydrophobicity characteristics or substitutions of residues with similar side-chain volume are well known. Isolated peptides comprising conservative amino acid substitutions can be tested to confirm that a desired activity, e.g. function as an agonist of GITR (for example, SEQ ID NO: 1 or SEQ ID NO: 2) is retained.

Amino acids can be grouped according to similarities in the properties of their side chains (in A. L. Lehninger, in Biochemistry, second ed., pp. 73-75, Worth Publishers, New York (1975)): (1) non-polar: Ala (A), Val (V), Leu (L), Ile (I), Pro (P), Phe (F), Trp (W), Met (M); (2) uncharged polar: Gly (G), Ser (S), Thr (T), Cys (C), Tyr (Y), Asn (N), Gln (Q); (3) acidic: Asp (D), Glu (E); (4) basic: Lys (K), Arg (R), His (H). Alternatively, naturally occurring residues can be divided into groups based on common side-chain properties: (1) hydrophobic: Norleucine, Met, Ala, Val, Leu, Ile, Phe, Trp; (2) neutral hydrophilic: Cys, Ser, Thr, Asn, Gln, Ala, Tyr, His, Pro, Gly; (3) acidic: Asp, Glu; (4) basic: His, Lys, Arg; (5) residues that influence chain orientation: Gly, Pro; (6) aromatic: Trp, Tyr, Phe, Pro, His, or hydroxyproline. Non-conservative substitutions will entail exchanging a member of one of these classes for another class.

Particularly preferred conservative substitutions for use in the variants described herein are as follows: Ala into Gly or into Ser; Arg into Lys; Asn into Gln or into His; Asp into Glu or into Asn; Cys into Ser; Gln into Asn; Glu into Asp; Gly into Ala or into Pro; His into Asn or into Gln; Ile into Leu or into Val; Leu into Ile or into Val; Lys into Arg, into Gln or into Glu; Met into Leu, into Tyr or into Ile; Phe into Met, into Leu or into Tyr; Ser into Thr; Thr into Ser; Trp into Tyr or into Phe; Tyr into Phe or into Trp; and/or Phe into Val, into Tyr, into Ile or into Leu. In general, conservative substitutions encompass residue exchanges with those of similar physicochemical properties (i.e. substitution of a hydrophobic residue for another hydrophobic amino acid).

Any cysteine residue not involved in maintaining the proper conformation of the isolated peptide as described herein can also be substituted, generally with serine, to improve the oxidative stability of the molecule and prevent aberrant crosslinking. Conversely, cysteine bond(s) can be added to the isolated peptide as described herein to improve its stability or facilitate multimerization.

As used herein, a “functional fragment” is a fragment or segment of a peptide comprising at least 3, at least 4 or at least 5 amino acids and which can function as agonists of GITR (for example, SEQ ID NO: 1 or SEQ ID NO: 2). A functional fragment can comprise conservative substitutions of the sequences disclosed herein so long as they preserve the function as an agonist of GITR (for example, SEQ ID NO: 1 or SEQ ID NO: 2). This can be tested by detecting an increase in function by at least 30%, at least 40% or at least 50% of that of the parent (e.g. original) version of the peptide.

To enhance stability, bioavailability, and/or delivery of the peptides into the cells, the peptides can be modified. For example, in some embodiments, an isolated peptide as described herein can comprise at least one peptide bond replacement. A single peptide bond or multiple peptide bonds, e.g. 2 bonds, 3 bonds, 4 bonds, 5 bonds, or 6 or more bonds, or all the peptide bonds can be replaced. An isolated peptide as described herein can comprise one type of peptide bond replacement or multiple types of peptide bond replacements, e.g. 2 types, 3 types, 4 types, 5 types, or more types of peptide bond replacements. Non-limiting examples of peptide bond replacements include urea, thiourea, carbamate, sulfonyl urea, trifluoroethylamine, ortho-(aminoalkyl)-phenylacetic acid, para-(aminoalkyl)-phenylacetic acid, meta-(aminoalkyl)-phenylacetic acid, thioamide, tetrazole, boronic ester, olefinic group, and derivatives thereof. In some embodiments, the peptides described herein (peptides comprising, consisting of or consisting essentially of the sequences set forth in SEQ ID NO: 1 or SEQ ID NO: 2) or a variants, derivatives, pharmaceutical equivalents, peptidomimetics or analogs thereof, are conjugated with agents that increase retention in the subject. Examples of agents that increase retention include but are not limited to cellulose, fatty acids, polyethylene glycol (PEG) or combinations thereof.

In some embodiments, an isolated peptide as described herein can comprise naturally occurring amino acids commonly found in polypeptides and/or proteins produced by living organisms, e.g. Ala (A), Val (V), Leu (L), Ile (I), Pro (P), Phe (F), Trp (W), Met (M), Gly (G), Ser (S), Thr (T), Cys (C), Tyr (Y), Asn (N), Gln (Q), Asp (D), Glu (E), Lys (K), Arg (R), and His (H). In some embodiments, an isolated peptide as described herein can comprise alternative amino acids. Non-limiting examples of alternative amino acids include, D-amino acids; beta-amino acids; homocysteine, phosphoserine, phosphothreonine, phosphotyrosine, hydroxyproline, gamma-carboxyglutamate; hippuric acid, octahydroindole-2-carboxylic acid, statine, 1,2,3,4,-tetrahydroisoquinoline-3-carboxylic acid, penicillamine (3-mercapto-D-valine), ornithine, citruline, alpha-methyl-alanine, para-benzoylphenylalanine, para-amino phenylalanine, p-fluorophenylalanine, phenylglycine, propargylglycine, sarcosine, and tert-butylglycine), diaminobutyric acid, 7-hydroxy-tetrahydroisoquinoline carboxylic acid, naphthylalanine, biphenylalanine, cyclohexylalanine, amino-isobutyric acid, norvaline, norleucine, tert-leucine, tetrahydroisoquinoline carboxylic acid, pipecolic acid, phenylglycine, homophenylalanine, cyclohexylglycine, dehydroleucine, 2,2-diethylglycine, 1-amino-1-cyclopentanecarboxylic acid, 1-amino-1-cyclohexanecarboxylic acid, amino-benzoic acid, amino-naphthoic acid, gamma-aminobutyric acid, difluorophenylalanine, nipecotic acid, alpha-amino butyric acid, thienyl-alanine, t-butylglycine, trifluorovaline; hexafluoroleucine; fluorinated analogs; azide-modified amino acids; alkyne-modified amino acids; cyano-modified amino acids; and derivatives thereof.

In some embodiments, an isolated peptide can be modified, e.g. a moiety can be added to one or more of the amino acids comprising the peptide. In some embodiments, an isolated peptide as described herein can comprise one or more moiety molecules, e.g. 1 or more moiety molecules per peptide, 2 or more moiety molecules per peptide, 5 or more moiety molecules per peptide, 10 or more moiety molecules per peptide or more moiety molecules per peptide. In some embodiments, an isolated peptide as described herein can comprise one more types of modifications and/or moieties, e.g. 1 type of modification, 2 types of modifications, 3 types of modifications or more types of modifications. Non-limiting examples of modifications and/or moieties include PEGylation; glycosylation; HESylation; ELPylation; lipidation; acetylation; amidation; end-capping modifications; cyano groups; phosphorylation; and cyclization. In some embodiments, an end-capping modification can comprise acetylation at the N-terminus, N-terminal acylation, and N-terminal formylation. In some embodiments, an end-capping modification can comprise amidation at the C-terminus, introduction of C-terminal alcohol, aldehyde, ester, and thioester moieties.

An isolated peptide as described herein can be coupled and or connected to a second functional molecule, peptide and/or polypeptide. In some embodiments, an isolated peptide as described herein is coupled to a targeting molecule. In some embodiments, an isolated peptide as described herein is coupled to a targeting molecule by expressing the peptide and the targeting molecule as a fusion peptide, optionally with a peptide linker sequence interposed between them. As used herein a “targeting molecule” can be any molecule, e.g. a peptide, antibody or fragment thereof, antigen, targeted liposome, or a small molecule that can bind to or be bound by a specific cell or tissue type.

In some embodiments, an isolated peptide as described herein can be a fusion peptide or polypeptide. A fusion polypeptide can comprise a peptide linker domain interposed between the first domain of the peptide comprising an amino acid sequence of the peptides described herein (SEQ ID NO: 1 or SEQ ID NO: 2), variants, functional fragments, prodrug, or analog thereof as described herein and at least a second domain of the fusion peptide. The first peptide domain can be the N-terminal domain or the C-terminal domain or an internal sequence in the case where the partner domain forms after fragment complementation of constituent parts. Methods of synthesizing or producing a fusion protein are well known to those of ordinary skill in the art. The term “fusion protein” as used herein refers to a recombinant protein of two or more proteins. Fusion proteins can be produced, for example, by a nucleic acid sequence encoding one protein is joined to the nucleic acid encoding another protein such that they constitute a single open-reading frame that can be translated in the cells into a single polypeptide harboring all the intended proteins. The order of arrangement of the proteins can vary. Fusion proteins can include an epitope tag or a half-life extender. Epitope tags include biotin, FLAG tag, c-myc, hemaglutinin, His6, digoxigenin, FITC, Cy3, Cy5, green fluorescent protein, V5 epitope tags, GST, β-galactosidase, AUl, AU5, and avidin. Half-life extenders include Fc domain and serum albumin.

In some embodiments, an isolated peptide as described herein (for example, peptides having the sequence set forth in SEQ ID NO: 1 or SEQ ID NO: 2 can be a pharmaceutically acceptable prodrug. As used herein, a “prodrug” refers to compounds that can be converted via some chemical or physiological process (e.g., enzymatic processes and metabolic hydrolysis) to a therapeutic agent. Thus, the term “prodrug” also refers to a precursor of a biologically active compound that is pharmaceutically acceptable. A prodrug may be inactive when administered to a subject, i.e. an ester, but is converted in vivo to an active compound, for example, by hydrolysis to the free carboxylic acid or free hydroxyl. The prodrug compound often offers advantages of solubility, tissue compatibility or delayed release in an organism. The term “prodrug” is also meant to include any covalently bonded carriers, which release the active compound in vivo when such prodrug is administered to a subject. Prodrugs of an active compound may be prepared by modifying functional groups present in the active compound in such a way that the modifications are cleaved, either in routine manipulation or in vivo, to the parent active compound. Prodrugs include compounds wherein a hydroxy, amino or mercapto group is bonded to any group that, when the prodrug of the active compound is administered to a subject, cleaves to form a free hydroxy, free amino or free mercapto group, respectively. Examples of prodrugs include, but are not limited to, acetate, formate and benzoate derivatives of an alcohol or acetamide, formamide and benzamide derivatives of an amine functional group in the active compound and the like. See Harper, “Drug Latentiation” in Jucker, ed. Progress in Drug Research 4:221-294 (1962); Morozowich et al, “Application of Physical Organic Principles to Prodrug Design” in E. B. Roche ed. Design of Biopharmaceutical Properties through Prodrugs and Analogs, APHA Acad. Pharm. Sci. 40 (1977); Bioreversible Carriers in Drug in Drug Design, Theory and Application, E. B. Roche, ed., APHA Acad. Pharm. Sci. (1987); Design of Prodrugs, H. Bundgaard, Elsevier (1985); Wang et al. “Prodrug approaches to the improved delivery of peptide drug” in Curr. Pharm. Design. 5(4):265-287 (1999); Pauletti et al. (1997) Improvement in peptide bioavailability: Peptidomimetics and Prodrug Strategies, Adv. Drug. Delivery Rev. 27:235-256; Mizen et al. (1998) “The Use of Esters as Prodrugs for Oral Delivery of (3-Lactam antibiotics,” Pharm. Biotech. ll,:345-365; Gaignault et al. (1996) “Designing Prodrugs and Bioprecursors I. Carrier Prodrugs,” Pract. Med. Chem. 671-696; Asgharnejad, “Improving Oral Drug Transport”, in Transport Processes in Pharmaceutical Systems, G. L. Amidon, P. I. Lee and E. M. Topp, Eds., Marcell Dekker, p. 185-218 (2000); Balant et al., “Prodrugs for the improvement of drug absorption via different routes of administration”, Eur. J. Drug Metab. Pharmacokinet., 15(2): 143-53 (1990); Balimane and Sinko, “Involvement of multiple transporters in the oral absorption of nucleoside analogues”, Adv. Drug Delivery Rev., 39(1-3): 183-209 (1999); Browne, “Fosphenytoin (Cerebyx)”, Clin. Neuropharmacol. 20(1): 1-12 (1997); Bundgaard, “Bioreversible derivatization of drugs—principle and applicability to improve the therapeutic effects of drugs”, Arch. Pharm. Chemi 86(1): 1-39 (1979); Bundgaard H. “Improved drug delivery by the prodrug approach”, Controlled Drug Delivery 17: 179-96 (1987); Bundgaard H. “Prodrugs as a means to improve the delivery of peptide drugs”, Arfv. Drug Delivery Rev. 8(1): 1-38 (1992); Fleisher et al. “Improved oral drug delivery: solubility limitations overcome by the use of prodrugs”, Arfv. Drug Delivery Rev. 19(2): 115-130 (1996); Fleisher et al. “Design of prodrugs for improved gastrointestinal absorption by intestinal enzyme targeting”, Methods Enzymol. 112 (Drug Enzyme Targeting, Pt. A): 360-81, (1985); Farquhar D, et al., “Biologically Reversible Phosphate-Protective Groups”, Pharm. Sci., 72(3): 324-325 (1983); Freeman S, et al., “Bioreversible Protection for the Phospho Group: Chemical Stability and Bioactivation of Di(4-acetoxy-benzyl) Methylphosphonate with Carboxyesterase,” Chem. Soc., Chem. Commun., 875-877 (1991); Friis and Bundgaard, “Prodrugs of phosphates and phosphonates: Novel lipophilic alphaacyloxyalkyl ester derivatives of phosphate- or phosphonate containing drugs masking the negative charges of these groups”, Eur. J. Pharm. Sci. 4: 49-59 (1996); Gangwar et al., “Pro-drug, molecular structure and percutaneous delivery”, Des. Biopharm. Prop. Prodrugs Analogs, [Symp.] Meeting Date 1976, 409-21. (1977); Nathwani and Wood, “Penicillins: a current review of their clinical pharmacology and therapeutic use”, Drugs 45(6): 866-94 (1993); Sinhababu and Thakker, “Prodrugs of anticancer agents”, Adv. Drug Delivery Rev. 19(2): 241-273 (1996); Stella et al., “Prodrugs. Do they have advantages in clinical practice?”, Drugs 29(5): 455-73 (1985); Tan et al. “Development and optimization of anti-HIV nucleoside analogs and prodrugs: A review of their cellular pharmacology, structure-activity relationships and pharmacokinetics”, Adv. Drug Delivery Rev. 39(1-3): 117-151 (1999); Taylor, “Improved passive oral drug delivery via prodrugs”, Adv. Drug Delivery Rev., 19(2): 131-148 (1996); Valentino and Borchardt, “Prodrug strategies to enhance the intestinal absorption of peptides”, Drug Discovery Today 2(4): 148-155 (1997); Wiebe and Knaus, “Concepts for the design of anti-HIV nucleoside prodrugs for treating cephalic HIV infection”, Adv. Drug Delivery Rev.: 39(1-3):63-80 (1999); Waller et al., “Prodrugs”, Br. J. Clin. Pharmac. 28: 497-507 (1989), which are incorporated by reference herein in their entireties.

In some embodiments, an isolated peptide as described herein can be a pharmaceutically acceptable solvate. The term “solvate” refers to an isolated peptide as described herein in the solid state, wherein molecules of a suitable solvent are incorporated in the crystal lattice. A suitable solvent for therapeutic administration is physiologically tolerable at the dosage administered. Examples of suitable solvents for therapeutic administration are ethanol and water. When water is the solvent, the solvate is referred to as a hydrate. In general, solvates are formed by dissolving the compound in the appropriate solvent and isolating the solvate by cooling or using an antisolvent. The solvate is typically dried or azeotroped under ambient conditions.

In some embodiments, an isolated peptide as described herein can be in a non-crystalline, i.e. amorphous solid form.

In one aspect, described herein is a vector comprising a nucleic acid encoding a peptide as described herein. The term “vector”, as used herein, refers to a nucleic acid construct designed for delivery to a host cell or for transfer between different host cells. As used herein, a vector can be viral or non-viral. The term “vector” encompasses any genetic element that is capable of replication when associated with the proper control elements and that can transfer gene sequences to cells. A vector can include, but is not limited to, a cloning vector, an expression vector, a plasmid, phage, transposon, cosmid, chromosome, virus, virion, etc. Many vectors useful for transferring exogenous genes into target mammalian cells are available. The vectors can be episomal, e.g., plasmids, virus derived vectors such cytomegalovirus, adenovirus, etc., or can be integrated into the target cell genome, through homologous recombination or random integration, e.g., retrovirus derived vectors such MMLV, HIV-1, ALV, etc. Many viral vectors are known in the art and can be used as carriers of a nucleic acid modulatory compound into the cell. For example, constructs containing the nucleic acid encoding a polypeptide can be integrated and packaged into non-replicating, defective viral genomes like Adenovirus, Adeno-associated virus (AAV), or Herpes simplex virus (HSV) or others, including retroviral and lentiviral vectors, for infection or transduction into cells. Alternatively, the construct can be incorporated into vectors capable of episomal replication, e.g. EPV and EBV vectors. The nucleic acid incorporated into the vector can be operatively linked to an expression control sequence such that the expression control sequence controls and regulates the transcription and translation of that polynucleotide sequence.

As used herein, the term “expression vector” refers to a vector that directs expression of an RNA or polypeptide from sequences linked to transcriptional regulatory sequences on the vector. The sequences expressed will often, but not necessarily, be heterologous to the cell. An expression vector can comprise additional elements, for example, the expression vector can have two replication systems, thus allowing it to be maintained in two organisms, for example in human cells for expression and in a prokaryotic host for cloning and amplification.

The term “transfection” as used herein to methods, such as chemical methods, to introduce exogenous nucleic acids, such as the nucleic acid sequences encoding a peptide as described herein into a cell. As used herein, the term transfection does not encompass viral-based methods of introducing exogenous nucleic acids into a cell. Methods of transfection include physical treatments (electroporation, nanoparticles, magnetofection), and chemical-based transfection methods. Chemical-based transfection methods include, but are not limited to those that use cyclodextrin, polymers, liposomes, nanoparticles, cationic lipids or mixtures thereof (e.g., DOPA, Lipofectamine and UptiFectin), and cationic polymers, such as DEAE-dextran or polyethylenimine.

As used herein, the term “viral vector” refers to a nucleic acid vector construct that includes at least one element of viral origin and has the capacity to be packaged into a viral vector particle. The viral vector can contain the nucleic acid encoding a peptide as described herein in place of non-essential viral genes. The vector and/or particle can be utilized for the purpose of transferring any nucleic acids into cells either in vitro or in vivo. Numerous forms of viral vectors are known in the art. The term “replication incompetent” when used in reference to a viral vector means the viral vector cannot further replicate and package its genomes. For example, when the cells of a subject are infected with replication incompetent recombinant adeno-associated virus (rAAV) virions, the heterologous (also known as transgene) gene is expressed in the patient's cells, but, the rAAV is replication defective (e.g., lacks accessory genes that encode essential proteins for packaging the virus) and viral particles cannot be formed in the patient's cells. The term “transduction” as used herein refers to the use of viral particles or viruses to introduce exogenous nucleic acids into a cell.

Retroviruses, such as lentiviruses, provide a convenient platform for delivery of nucleic acid sequences encoding an agent of interest. A selected nucleic acid sequence can be inserted into a vector and packaged in retroviral particles using techniques known in the art. The recombinant virus can then be isolated and delivered to cells, e.g. in vitro or ex vivo. Retroviral systems are well known in the art and are described in, for example, U.S. Pat. No. 5,219,740; Kurth and Bannert (2010) “Retroviruses: Molecular Biology, Genomics and Pathogenesis” Calster Academic Press (ISBN:978-1-90455-55-4); and Hu and Pathak Pharmacological Reviews 2000 52:493-512; which are incorporated by reference herein in their entirety.

In some embodiments, a nucleotide sequence of interest is inserted into an adenovirus-based expression vector. Unlike retroviruses, which integrate into the host genome, adenoviruses persist extrachromosomally thus minimizing the risks associated with insertional mutagenesis (Haj-Ahmad and Graham (1986) J. Virol. 57:267-74; Bett et al. (1993) J. Virol. 67:5911-21; Mittereder et al. (1994) Human Gene Therapy 5:717-29; Seth et al. (1994) J. Virol. 68:933-40; Barr et al. (1994) Gene Therapy 1:51-58; Berkner, K. L. (1988) BioTechniques 6:616-29; and Rich et al. (1993) Human Gene Therapy 4:461-76). Adenoviral vectors have several advantages in gene therapy. They infect a wide variety of cells, have a broad host-range, exhibit high efficiencies of infectivity, direct expression of heterologous sequences at high levels, and achieve long-term expression of those sequences in vivo. The virus is fully infective as a cell-free virion so injection of producer cell lines is not necessary. With regard to safety, adenovirus is not associated with severe human pathology, and the recombinant vectors derived from the virus can be rendered replication defective by deletions in the early-region 1 (“E1”) of the viral genome. Adenovirus can also be produced in large quantities with relative ease. For all these reasons vectors derived from human adenoviruses, in which at least the E1 region has been deleted and replaced by a gene of interest, have been used extensively for gene therapy experiments in the pre-clinical and clinical phase. Adenoviral vectors for use with the compositions and methods described herein can be derived from any of the various adenoviral serotypes, including, without limitation, any of the over 40 serotype strains of adenovirus, such as serotypes 2, 5, 12, 40, and 41. The adenoviral vectors of used in the methods described herein are generally replication-deficient and contain the sequence of interest under the control of a suitable promoter. For example, U.S. Pat. No. 6,048,551, incorporated herein by reference in its entirety, describes replication-deficient adenoviral vectors that include a human gene under the control of the Rous Sarcoma Virus (RSV) promoter. Other recombinant adenoviruses of various serotypes, and comprising different promoter systems, can be created by those skilled in the art. See, e.g., U.S. Pat. No. 6,306,652, incorporated herein by reference in its entirety. Other useful adenovirus-based vectors for delivery of nucleic acid sequences include, but are not limited to: “minimal” adenovirus vectors as described in U.S. Pat. No. 6,306,652, which retain at least a portion of the viral genome required for encapsidation (the encapsidation signal), as well as at least one copy of at least a functional part or a derivative of the ITR; and the “gutless” (helper-dependent) adenovirus in which the vast majority of the viral genome has been removed and which produce essentially no viral proteins, such vectors can permit gene expression to persist for over a year after a single administration (Wu et al. (2001) Anesthes. 94:1119-32; Parks (2000) Clin. Genet. 58:1-11; Tsai et al. (2000) Curr. Opin. Mol. Ther. 2:515-23).

In some embodiments, a nucleotide sequence encoding a peptide as described herein is inserted into an adeno-associated virus-based expression vector. AAV is a parvovirus which belongs to the genus Dependovirus and has several features not found in other viruses. AAV can infect a wide range of host cells, including non-dividing cells. AAV can infect cells from different species. AAV has not been associated with any human or animal disease and does not appear to alter the biological properties of the host cell upon integration. Indeed, it is estimated that 80-85% of the human population has been exposed to the virus. Finally, AAV is stable at a wide range of physical and chemical conditions, facilitating production, storage and transportation. AAV is a helper-dependent virus; that is, it requires co-infection with a helper virus (e.g., adenovirus, herpesvirus or vaccinia) in order to form AAV virions in the wild. In the absence of co-infection with a helper virus, AAV establishes a latent state in which the viral genome inserts into a host cell chromosome, but infectious virions are not produced. Subsequent infection by a helper virus rescues the integrated genome, allowing it to replicate and package its genome into infectious AAV virions. While AAV can infect cells from different species, the helper virus must be of the same species as the host cell. Thus, for example, human AAV will replicate in canine cells co-infected with a canine adenovirus. Adeno-associated virus (AAV) has been used with success in gene therapy. AAV has been engineered to deliver genes of interest by deleting the internal nonrepeating portion of the AAV genome (i.e., the rep and cap genes) and inserting a heterologous sequence (in this case, the sequence encoding the agent) between the ITRs. The heterologous sequence is typically functionally linked to a heterologous promoter (constitutive, cell-specific, or inducible) capable of driving expression in the patient's target cells under appropriate conditions. Recombinant AAV virions comprising a nucleic acid sequence encoding an agent of interest can be produced using a variety of art-recognized techniques, as described in U.S. Pat. Nos. 5,139,941; 5,622,856; 5,139,941; 6,001,650; and 6,004,797, the contents of each of which are incorporated by reference herein in their entireties. Vectors and cell lines necessary for preparing helper virus-free rAAV stocks are commercially available as the AAV Helper-Free System (Catalog No. 240071) (Agilent Technologies, Santa Clara, Calif.).

Additional viral vectors useful for delivering nucleic acid molecules encoding a peptide as described herein include those derived from the pox family of viruses, including vaccinia virus and avian poxvirus. Alternatively, avipoxviruses, such as the fowlpox and canarypox viruses, can be used to deliver the genes. The use of avipox vectors in cells of human and other mammalian species is advantageous with regard to safety because members of the avipox genus can only productively replicate in susceptible avian species. Methods for producing recombinant avipoxviruses are known in the art and employ genetic recombination, see, e.g., WO 91/12882; WO 89/03429; and WO 92/03545.

Molecular conjugate vectors, such as the adenovirus chimeric vectors, can also be used for delivery of sequence encoding a peptide as described herein (Michael et al. (1993) J. Biol. Chem. 268:6866-69 and Wagner et al. (1992) Proc. Natl. Acad. Sci. USA 89:6099-6103). Members of the Alphavirus genus, for example the Sindbis and Semliki Forest viruses, can also be used as viral vectors for delivering a nucleic acid sequence (See, e.g., Dubensky et al. (1996) J. Virol. 70:508-19; WO 95/07995; WO 96/17072).

In some embodiments, the vector further comprises a signal peptide operably linked to the peptide. Signal peptides are terminally (usually N-terminally) located peptide sequences that provide for passage of the protein into or through a membrane. Different signal peptides can be of use in different applications. For example, as regards a cellular system for the production of isolated peptides as described herein, a secretory signal peptide can permit increased yields and ease of purification. As a further example, as regards cells which produce peptides as described herein and which are administered for therapeutic purposes to a subject, multiple signal peptides, e.g. a peptide signaling for secretion from the first cell, a peptide signaling for internalization by a second cell, and a final peptide signaling for nuclear localization can increase the amount of peptide reaching the target environment. As a further example, as regards, e.g. gene therapy applications, a peptide signaling for nuclear localization can increase the amount of peptide reaching the target environment. Signal peptides are known in the art. Non-limiting examples of nuclear localization signal (NLS) peptides for use in mammalian cells include; the SV40 large T-antigen NLS; the nucleoplasmin NLS; the K-K/R-X-K/R consensus NLS. Additional signal peptides are known in the art and the choice of signal peptide can be influenced by the cell type, growth conditions, and the desired destination of the peptide.

In one aspect, described herein is a cell expressing a vector comprising a nucleic acid encoding a peptide as described herein. In some embodiments, the cell expressing a vector as described herein is a cell suitable for the production of polypeptides. A cell suitable for the production of polypeptides can be a prokaryotic or eukaryotic cell, e.g. bacteria, virus, yeast, fungi, mammalian cells, insect cells, plant cells, and the like. By way of non-limiting example, cells for the production of proteins are commercially available, e.g. bacterial cells (BL21 derived cells—Cat. No. 60401-1, Lucigen; Middleton, Wis. and mammalian cells (293 F cells—Cat. No. 11625-019, Invitrogen; Grand Island, N.Y.).

Recombinant molecules, e.g. vectors as described herein, can be introduced into cells via transformation, particularly transduction, conjugation, lipofection, protoplast fusion, mobilization, particle bombardment, electroporation (Neumann et al., “Gene Transfer into Mouse Lyoma Cells by Electroporation in High Electric Fields,” EMBO J. 1(7):841-845 (1982); Wong et al., “Electric Field Mediated Gene Transfer,” Biochem Biophys Res Commun 107(2):584-587 (1982); Potter et al., “Enhancer-dependent Expression of Human Kappa Immunoglobulin Genes Introduced into Mouse pre-B Lymphocytes by Electroporation,” Proc. Natl. Acad. Sci. USA 81(22):7161-7165 (1984), which are hereby incorporated by reference in their entirety), polyethylene glycol-mediated DNA uptake (Joseph Sambrook & David W. Russell, Molecular Cloning: A Laboratory Manual cp. 16 (2d ed. 1989), which is hereby incorporated by reference in its entirety), or fusion of protoplasts with other entities (e.g., minicells, cells, lysosomes, or other fusible lipid-surfaced bodies that contain the chimeric gene) (Fraley et al., “Liposome-mediated Delivery of Tobacco Mosaic Virus RNA into Tobacco Protoplasts: A Sensitive Assay for Monitoring Liposome-protoplast Interactions,” Proc. Natl. Acad. Sci. USA, 79(6):1859-1863 (1982), which is hereby incorporated by reference in its entirety). The host cell is then cultured in a suitable medium, and under conditions suitable for expression of the protein or polypeptide of interest. After cultivation, the cell is disrupted by physical or chemical means, and the protein or polypeptide purified from the resultant crude extract. Alternatively, cultivation may include conditions in which the protein or polypeptide is secreted into the growth medium of the recombinant host cell, and the protein or polypeptide is isolated from the growth medium. Alternative methods may be used as suitable.

The peptides can also be attached to adjuvants. The term “adjuvant” refers to a compound or mixture that enhances the immune response and/or promotes the proper rate of absorption following inoculation, and, as used herein, encompasses any uptake-facilitating agent. Non-limiting examples of adjuvants include, chemokines (e.g., defensins, HCC-1, HCC4, MCP-1, MCP-3, MCP4, MIP-1α, MIP-1β, MIP-1δ, MIP-3α, MIP-2, RANTES); other ligands of chemokine receptors (e.g., CCR1, CCR-2, CCR-5, CCR6, CXCR-1); cytokines (e.g., IL-1β, IL-2, IL-4, IL-5, IL-6, IL-7, IL-8, IL-10, IL-12, IL-13, IL-15, IL-17 (A-F), IL-18; IFNα, IFN-γ; TNF-α; GM-CSF); TGF)-β; FLT-3 ligand; CD40 ligand; other ligands of receptors for those cytokines; Th1 cytokines including, without limitation, IFN-γ, IL-2, IL-12, IL-18, and TNF; Th2 cytokines including, without limitation, IL-4, IL-5, IL-10, and IL-13; and Th17 cytokines including, without limitation, IL-17 (A through F), IL-23, TGF-β and IL-6; immunostimulatory CpG motifs in bacterial DNA or oligonucleotides; derivatives of lipopolysaccharides such as monophosphoryl lipid A (MPL); muramyl dipeptide (MDP) and derivatives thereof (e.g., murabutide, threonyl-MDP, muramyl tripeptide, N-acetyl-muramyl-L-threonyl-D-isoglutamine (thr-MDP); N-acetyl-nor-muramyl-L-alanyl-D-isoglutamine (CGP 11637, referred to as nor-MDP); N-acetylmuramyl-L-alanyl-D-isoglutaminyl-L-alanine-2-(1′-2′-dipalmitoyl-sn-glycero-3hydroxyphosphoryloxy)-ethylamine (CGP 19835A, referred to as MTP-PE)); MF59 (see Int'l Publication No. WO 90/14837); poly[di(carboxylatophenoxy)phosphazene] (PCPP polymer; Virus Research Institute, USA); RIBI (GSK), which contains three components extracted from bacteria, monophosphoryl lipid A, trehalose dimycolate and cell wall skeleton (MPL+TDM+CWS) in a 2% squalene/Tween 80 emulsion; OM-174 (a glucosamine disaccharide related to lipid A; OM Pharma SA, Meyrin, Switzerland); heat shock proteins and derivatives thereof; Leishmania homologs of elF4a and derivatives thereof, bacterial ADP-ribosylating exotoxins and derivatives thereof (e.g., genetic mutants, A and/or B subunit-containing fragments, chemically toxoided versions); chemical conjugates or genetic recombinants containing bacterial ADP-ribosylating exotoxins or derivatives thereof; C3d tandem array; lipid A and derivatives thereof (e.g., monophosphoryl or diphosphoryl lipid A, lipid A analogs, AGP, AS02, AS04, DC-Chol, Detox, OM-174); ISCOMS and saponins (e.g., Quil A, QS-21, Stimulon® (Cambridge Bioscience, Worcester, Mass.)); squalene; superantigens; or salts (e.g., aluminum hydroxide or phosphate, calcium phosphate). See also Nohria et al. Biotherapy, 7:261-269, 1994; Richards et al., in Vaccine Design, Eds. Powell et al., Plenum Press, 1995; and Pashine et al., Nature Medicine, 11:S63-S68, 4/2005) for other useful adjuvants. Further examples of adjuvants can include the RIBI adjuvant system (Ribi Inc., Hamilton, Mont.), alum, mineral gels such as aluminum hydroxide gel, oil-in-water emulsions, water-in-oil emulsions such as, e.g., Freund's complete and incomplete adjuvants, Block co-polymer (CytRx, Atlanta Ga.), QS-21 (Cambridge Biotech Inc., Cambridge Mass.), and SAF-M (Chiron, Emeryville Calif.), AMPHIGEN® adjuvant, saponin, Quil A or other saponin fraction, monophosphoryl lipid A, and Avridine lipid-amine adjuvant, and METASTIM®. Other suitable adjuvants can include, for example, surface active substances such as lysolecithin, pluronic polyols, polyanions, peptides, oil or hydrocarbon emulsions, keyhole limpet hemocyanins, dinitrophenol, and others.

In some embodiment, cell may be genetically engineered to express the peptides described herein and the genetically engineered cells may be used for cell therapy. In some embodiment, cell therapy is also considered as ex vivo therapy. Examples of cells that may be used include but are not limited to, dendritic cells, T-lymphocytes (T-cells), naïve T cells (T_{N}), memory T cells (for example, central memory T cells (T_{CM}), effector memory cells (T_{EM})), natural killer cells, hematopoietic stem cells and/or pluripotent embryonic/induced stem cells capable of giving rise to therapeutically relevant progeny. In an embodiment, the genetically engineered cells are autologous cells. By way of example, individual T-cells of the invention may be CD4+/CD8−, CD4−/CD8+, CD4−/CD8− or CD4+/CD8+. The T-cells may be a mixed population of CD4+/CD8− and CD4−/CD8+ cells or a population of a single clone. CD4+ T-cells may produce IL-2, IFNγ, TNFα and other T-cell effector cytokines when co-cultured in vitro with cells expressing the peptides (for example CD20+ and/or CD19+ tumor cells). CD8⁺ T-cells may lyse antigen-specific target cells when co-cultured in vitro with the target cells. In some embodiments, T cells may be any one or more of CD45RA⁺CD62L⁺ naïve cells, CD45RO⁺ CD62L⁺ central memory cells, CD62L⁻ effector memory cells or a combination thereof (Berger et al., Adoptive transfer of virus-specific and tumor-specific T cell immunity. Curr Opin Immunol 2009 21(2)224-232).

In some embodiments, tolerized antigen presenting cells may be used in cell therapy. Examples include B cells, dendritic cells, macrophages and the like. The cells may be of any origin, including from humans. The cells may be tolerized using the peptides described herein. In some embodiments, the cells are tolerized in the presence of cytokines.

In some embodiments, the cell producing the peptide as described herein can be administered to a subject, e.g. for treating, inhibiting, reducing the severity of and/or slow progression of cancer (SEQ ID NO: 1 and/or SEQ ID NO: 2).

In some embodiments, nanoparticles containing the peptide as described herein can be administrated to a subject. In some embodiments, the nanoparticles for use with the peptides described herein may be as described in Levine et al., Polymersomes: A new multi-functional tool for cancer diagnosis and therapy. Methods 2008 Vol 46 pg 25-32 or as described in S Jain, et al., Gold nanoparticles as novel agents for cancer therapy. Br J Radiol. 2012 February; 85(1010): 101-113.

In some embodiments, the cell expressing a vector encoding a peptide as described herein can be a cell of a subject, e.g. a subject administered gene therapy for the treatment, inhibition, reduction of severity and/or slow progression of diabetes (such as type 2 diabetes mellitus). Vectors for gene therapy can comprise viral or non-viral vectors as described elsewhere herein.

Pharmaceutical Compositions

In various embodiments, the present invention provides a pharmaceutical composition, comprising: compositions having one or more compounds of the invention; and a pharmaceutically acceptable carrier. In one embodiment, the compound is one or more agonist of GITR (for example, peptides having the sequence set forth in SEQ ID NO: 1, SEQ ID NO: 2 or variants, derivatives or functional equivalents thereof, or compounds of Formula I). In another embodiment, the compound is one or more antagonist of GITR (for example, compounds compounds of Formula II).

For administration to a subject, the compositions described herein can be provided in pharmaceutically acceptable compositions. These pharmaceutically acceptable compositions comprise a peptide and/or a compound capable of functioning as an agonist or antagonist of GITR as described herein formulated together with one or more pharmaceutically acceptable carriers (additives) and/or diluents. As described in detail below, the pharmaceutical compositions of the present invention can be specially formulated for administration in solid or liquid form, including those adapted for the following: (1) oral administration, for example, drenches (aqueous or non-aqueous solutions or suspensions), gavages, lozenges, dragees, capsules, pills, tablets (e.g., those targeted for buccal, sublingual, and systemic absorption), boluses, powders, granules, pastes for application to the tongue; (2) parenteral administration, for example, by subcutaneous, intramuscular, intravenous or epidural injection as, for example, a sterile solution or suspension, or sustained-release formulation; (3) topical application, for example, as a cream, ointment, or a controlled-release patch or spray applied to the skin; (4) intrarectally, for example, as a pessary, cream or foam; (5) sublingually; (6) ocularly; (7) transdermally; (8) transmucosally; or (9) nasally. Additionally, compounds can be implanted into a patient or injected using a drug delivery system. See, for example, Urquhart, et al., Ann. Rev. Pharmacol. Toxicol. 24: 199-236 (1984); Lewis, ed. “Controlled Release of Pesticides and Pharmaceuticals” (Plenum Press, New York, 1981); U.S. Pat. No. 3,773,919; and U.S. Pat. No. 35 3,270,960, contents of all of which are herein incorporated by reference.

As used here, the term “pharmaceutically acceptable” refers to those compounds, materials, compositions, and/or dosage forms which are, within the scope of sound medical judgment, suitable for use in contact with the tissues of human beings and animals without excessive toxicity, irritation, allergic response, or other problem or complication, commensurate with a reasonable benefit/risk ratio.

As used here, the term “pharmaceutically-acceptable carrier” means a pharmaceutically-acceptable material, composition or vehicle, such as a liquid or solid filler, diluent, excipient, manufacturing aid (e.g., lubricant, talc magnesium, calcium or zinc stearate, or steric acid), or solvent encapsulating material, involved in carrying or transporting the subject compound from one organ, or portion of the body, to another organ, or portion of the body. Each carrier must be “acceptable” in the sense of being compatible with the other ingredients of the formulation and not injurious to the patient. Some examples of materials which can serve as pharmaceutically-acceptable carriers include: (1) sugars, such as lactose, glucose and sucrose; (2) starches, such as corn starch and potato starch; (3) cellulose, and its derivatives, such as sodium carboxymethyl cellulose, methylcellulose, ethyl cellulose, microcrystalline cellulose and cellulose acetate; (4) powdered tragacanth; (5) malt; (6) gelatin; (7) lubricating agents, such as magnesium stearate, sodium lauryl sulfate and talc; (8) excipients, such as cocoa butter and suppository waxes; (9) oils, such as peanut oil, cottonseed oil, safflower oil, sesame oil, olive oil, corn oil and soybean oil; (10) glycols, such as propylene glycol; (11) polyols, such as glycerin, sorbitol, mannitol and polyethylene glycol (PEG); (12) esters, such as ethyl oleate and ethyl laurate; (13) agar; (14) buffering agents, such as magnesium hydroxide and aluminum hydroxide; (15) alginic acid; (16) pyrogen-free water; (17) isotonic saline; (18) Ringer's solution; (19) ethyl alcohol; (20) pH buffered solutions; (21) polyesters, polycarbonates and/or polyanhydrides; (22) bulking agents, such as polypeptides and amino acids (23) serum component, such as serum albumin, HDL and LDL; (22) C₂-C₁₂ alcohols, such as ethanol; and (23) other non-toxic compatible substances employed in pharmaceutical formulations. Wetting agents, coloring agents, release agents, coating agents, sweetening agents, flavoring agents, perfuming agents, preservative and antioxidants can also be present in the formulation. The terms such as “excipient”, “carrier”, “pharmaceutically acceptable carrier” or the like are used interchangeably herein.

The pharmaceutical compositions according to the invention can also be encapsulated, tableted or prepared in an emulsion or syrup for oral administration. Pharmaceutically acceptable solid or liquid carriers may be added to enhance or stabilize the composition, or to facilitate preparation of the composition. Liquid carriers include syrup, peanut oil, olive oil, glycerin, saline, alcohols and water. Solid carriers include starch, lactose, calcium sulfate, dihydrate, terra alba, magnesium stearate or stearic acid, talc, pectin, acacia, agar or gelatin. The carrier may also include a sustained release material such as glyceryl monostearate or glyceryl distearate, alone or with a wax.

The pharmaceutical compositions are made following the conventional techniques of pharmacy involving dry milling, mixing, and blending for powder forms; milling, mixing, granulation, and compressing, when necessary, for tablet forms; or milling, mixing and filling for hard gelatin capsule forms. When a liquid carrier is used, the preparation will be in the form of a syrup, elixir, emulsion or an aqueous or non-aqueous suspension. Such a liquid formulation may be administered directly p.o. or filled into a soft gelatin capsule.

Before administration to patients, formulants may be added to the composition. A liquid formulation may be preferred. For example, these formulants may include oils, polymers, vitamins, carbohydrates, amino acids, salts, buffers, albumin, surfactants, bulking agents or combinations thereof.

Carbohydrate formulants include sugar or sugar alcohols such as monosaccharides, disaccharides, or polysaccharides, or water soluble glucans. The saccharides or glucans can include fructose, dextrose, lactose, glucose, mannose, sorbose, xylose, maltose, sucrose, dextran, pullulan, dextrin, alpha and beta cyclodextrin, soluble starch, hydroxethyl starch and carboxymethylcellulose, or mixtures thereof. “Sugar alcohol” is defined as a C4 to C8 hydrocarbon having an —OH group and includes galactitol, inositol, mannitol, xylitol, sorbitol, glycerol, and arabitol. These sugars or sugar alcohols mentioned above may be used individually or in combination. There is no fixed limit to amount used as long as the sugar or sugar alcohol is soluble in the aqueous preparation. In one embodiment, the sugar or sugar alcohol concentration is between 1.0 w/v % and 7.0 w/v %, more preferable between 2.0 and 6.0 w/v %.

Amino acids formulants include levorotary (L) forms of carnitine, arginine, and betaine; however, other amino acids may be added.

Polymers formulants include polyvinylpyrrolidone (PVP) with an average molecular weight between 2,000 and 3,000, or polyethylene glycol (PEG) with an average molecular weight between 3,000 and 5,000.

It is also preferred to use a buffer in the composition to minimize pH changes in the solution before lyophilization or after reconstitution. Most any physiological buffer may be used including but not limited to citrate, phosphate, succinate, and glutamate buffers or mixtures thereof. In some embodiments, the concentration is from 0.01 to 0.3 molar. Surfactants that can be added to the formulation are shown in EP Nos. 270,799 and 268,110.

Another drug delivery system for increasing circulatory half-life is the liposome. Methods of preparing liposome delivery systems are discussed in Gabizon et al., Cancer Research (1982) 42:4734; Cafiso, Biochem Biophys Acta (1981) 649:129; and Szoka, Ann Rev Biophys Eng (1980) 9:467. Other drug delivery systems are known in the art and are described in, e.g., Poznansky et al., DRUG DELIVERY SYSTEMS (R. L. Juliano, ed., Oxford, N.Y. 1980), pp. 253-315; M. L. Poznansky, Pharm Revs (1984) 36:277.

After the liquid pharmaceutical composition is prepared, it may be lyophilized to prevent degradation and to preserve sterility. Methods for lyophilizing liquid compositions are known to those of ordinary skill in the art. Just prior to use, the composition may be reconstituted with a sterile diluent (Ringer's solution, distilled water, or sterile saline, for example) which may include additional ingredients. Upon reconstitution, the composition is administered to subjects using those methods that are known to those skilled in the art.

The compositions of the invention may be sterilized by conventional, well-known sterilization techniques. The resulting solutions may be packaged for use or filtered under aseptic conditions and lyophilized, the lyophilized preparation being combined with a sterile solution prior to administration. The compositions may contain pharmaceutically-acceptable auxiliary substances as required to approximate physiological conditions, such as pH adjusting and buffering agents, tonicity adjusting agents and the like, for example, sodium acetate, sodium lactate, sodium chloride, potassium chloride, calcium chloride, and stabilizers (e.g., 1-20% maltose, etc.).

The phrase “therapeutically effective amount” as used herein means that amount of an agent, compound, material, or composition comprising the same which is effective for producing some desired therapeutic effect in at least a sub-population of cells in an animal at a reasonable benefit/risk ratio applicable to a medical treatment. Determination of a therapeutically effective amount is well within the capability of those skilled in the art. Generally, a therapeutically effective amount can vary with the subject's history, age, condition, well as the severity and type of the medical condition in the subject, and administration of

The amount of the composition comprising a peptide and/or a compound capable of functioning as an agonist or antagonist of GITR as described herein that can be combined with a carrier material to produce a single dosage form will generally be that amount of the agent that produces a therapeutic effect. Generally out of one hundred percent, this amount will range from about 0.01% to 99% of agent, preferably from about 5% to about 70%, most preferably from 10% to about 30%.

Toxicity and therapeutic efficacy can be determined by standard pharmaceutical procedures in cell cultures or experimental animals, e.g., for determining the LD₅₀ (the dose lethal to 50% of the population) and the ED₅₀ (the dose therapeutically effective in 50% of the population). The dose ratio between toxic and therapeutic effects is the therapeutic index and it can be expressed as the ratio LD₅₀/ED₅₀. Compositions that exhibit large therapeutic indices are preferred.

As used herein, the term ED denotes effective dose and is used in connection with animal models. The term EC denotes effective concentration and is used in connection with in vitro models.

The data obtained from the cell culture assays and animal studies can be used in formulating a range of dosage for use in humans. The dosage of such compounds lies preferably within a range of circulating concentrations that include the ED₅₀ with little or no toxicity. The dosage may vary within this range depending upon the dosage form employed and the route of administration utilized.

The therapeutically effective dose can be estimated initially from cell culture assays. A dose may be formulated in animal models to achieve a circulating plasma concentration range that includes the IC₅₀ (i.e., the concentration of the therapeutic which achieves a half-maximal inhibition of symptoms) as determined in cell culture. Levels in plasma can be measured, for example, by high performance liquid chromatography. The effects of any particular dosage can be monitored by a suitable bioassay.

The dosage can be determined by a physician and adjusted, as necessary, to suit observed effects of the treatment. Generally, the compositions are administered so that the agent is given at a dose from 1 μg/kg to 150 mg/kg, 1 μg/kg to 100 mg/kg, 1 μg/kg to 50 mg/kg, 1 μg/kg to 20 mg/kg, 1 μg/kg to 10 mg/kg, 1 μg/kg to 1 mg/kg, 100 μg/kg to 100 mg/kg, 100 μg/kg to 50 mg/kg, 100 μg/kg to 20 mg/kg, 100 μg/kg to 10 mg/kg, 100 μg/kg to 1 mg/kg, 1 mg/kg to 100 mg/kg, 1 mg/kg to 50 mg/kg, 1 mg/kg to 20 mg/kg, 1 mg/kg to 10 mg/kg, 10 mg/kg to 100 mg/kg, 10 mg/kg to 50 mg/kg, or 10 mg/kg to 20 mg/kg. It is to be understood that ranges given here include all intermediate ranges, for example, the range 1 tmg/kg to 10 mg/kg includes 1 mg/kg to 2 mg/kg, 1 mg/kg to 3 mg/kg, 1 mg/kg to 4 mg/kg, 1 mg/kg to 5 mg/kg, 1 mg/kg to 6 mg/kg, 1 mg/kg to 7 mg/kg, 1 mg/kg to 8 mg/kg, 1 mg/kg to 9 mg/kg, 2 mg/kg to 10 mg/kg, 3 mg/kg to 10 mg/kg, 4 mg/kg to 10 mg/kg, 5 mg/kg to 10 mg/kg, 6 mg/kg to 10 mg/kg, 7 mg/kg to 10 mg/kg, 8 mg/kg to 10 mg/kg, 9 mg/kg to 10 mg/kg, and the like. It is to be further understood that the ranges intermediate to the given above are also within the scope of this invention, for example, in the range 1 mg/kg to 10 mg/kg, dose ranges such as 2 mg/kg to 8 mg/kg, 3 mg/kg to 7 mg/kg, 4 mg/kg to 6 mg/kg, and the like.

In some embodiments, the compositions are administered at a dosage so that agent or a metabolite thereof has an in vivo concentration of less than 500 nM, less than 400 nM, less than 300 nM, less than 250 nM, less than 200 nM, less than 150 nM, less than 100 nM, less than 50 nM, less than 25 nM, less than 20, nM, less than 10 nM, less than 5 nM, less than 1 nM, less than 0.5 nM, less than 0.1 nM, less than 0.05, less than 0.01, nM, less than 0.005 nM, less than 0.001 nM after 15 mins, 30 mins, 1 hr, 1.5 hrs, 2 hrs, 2.5 hrs, 3 hrs, 4 hrs, 5 hrs, 6 hrs, 7 hrs, 8 hrs, 9 hrs, 10 hrs, 11 hrs, 12 hrs or more of time of administration.

With respect to duration and frequency of treatment, it is typical for skilled clinicians to monitor subjects in order to determine when the treatment is providing therapeutic benefit, and to determine whether to increase or decrease dosage, increase or decrease administration frequency, discontinue treatment, resume treatment or make other alteration to treatment regimen. The dosing schedule can vary from once a week to daily depending on a number of clinical factors, such as the subject's sensitivity to the polypeptides. The desired dose can be administered every day or every third, fourth, fifth, or sixth day. The desired dose can be administered at one time or divided into subdoses, e.g., 2-4 subdoses and administered over a period of time, e.g., at appropriate intervals through the day or other appropriate schedule. Such sub-doses can be administered as unit dosage forms. In some embodiments of the aspects described herein, administration is chronic, e.g., one or more doses daily over a period of weeks or months. Examples of dosing schedules are administration daily, twice daily, three times daily or four or more times daily over a period of 1 week, 2 weeks, 3 weeks, 4 weeks, 1 month, 2 months, 3 months, 4 months, 5 months, or 6 months or more.

“Contacting” as used here with reference to contacting a cell with an agent (e.g., a compound disclosed herein) refers to any method that is suitable for placing the agent on, in or adjacent to a target cell. For example, when the cells are in vitro, contact the cells with the agent can comprise adding the agent to culture medium containing the cells. For example, when the cells are in vivo, contacting the cells with the agent can comprise administering the agent to the subject.

As used herein, the term “administering” refers to the placement of an agent or a composition as disclosed herein into a subject by a method or route which results in at least partial localization of the agents or composition at a desired site such that a desired effect is produced. Routes of administration suitable for the methods of the invention include both local and systemic administration. Generally, local administration results in more of the composition being delivered to a specific location as compared to the entire body of the subject, whereas, systemic administration results in delivery to essentially the entire body of the subject.

“Route of administration” may refer to any administration pathway known in the art, including but not limited to oral, topical, aerosol, nasal, via inhalation, anal, intra-anal, peri-anal, transmucosal, transdermal, parenteral, enteral, or local. “Parenteral” refers to a route of administration that is generally associated with injection, including intratumoral, intracranial, intraventricular, intrathecal, epidural, intradural, intraorbital, infusion, intracapsular, intracardiac, intradermal, intramuscular, intraperitoneal, intrapulmonary, intraspinal, intrasternal, intrathecal, intravascular, intravenous, intraarterial, subarachnoid, subcapsular, subcutaneous, transmucosal, or transtracheal. Via the parenteral route, the agent or composition may be in the form of solutions or suspensions for infusion or for injection, or as lyophilized powders. Via the enteral route, the agent or composition can be in the form of capsules, gel capsules, tablets, sugar-coated tablets, syrups, suspensions, solutions, powders, granules, emulsions, microspheres, nanoparticles comprised of proteineous or non-proteineous components or nanospheres or lipid vesicles or polymer vesicles allowing controlled release. Via the topical route, the agent or composition can be in the form of aerosol, lotion, cream, gel, ointment, suspensions, solutions or emulsions. In an embodiment, agent or composition may be provided in a powder form and mixed with a liquid, such as water, to form a beverage. In accordance with the present invention, “administering” can be self-administering. For example, it is considered as “administering” that a subject consumes a composition as disclosed herein.

Exemplary modes of administration include, but are not limited to, injection, infusion, instillation, inhalation, or ingestion. “Injection” includes, without limitation, intravenous, intramuscular, intraarterial, intrathecal, intraventricular, intracapsular, intraorbital, intracardiac, intradermal, intraperitoneal, transtracheal, subcutaneous, subcuticular, intraarticular, sub capsular, subarachnoid, intraspinal, intracerebro spinal, and intrasternal injection and infusion. In some embodiments of the various aspects described herein, the compositions are administered by intravenous infusion or injection.

A “pharmaceutically acceptable salt”, as used herein, is intended to encompass any compound described herein that is utilized in the form of a salt thereof, especially where the salt confers on the compound improved pharmacokinetic properties as compared to the free form of compound or a different salt form of the compound. The pharmaceutically acceptable salt form can also initially confer desirable pharmacokinetic properties on the compound that it did not previously possess, and may even positively affect the pharmacodynamics of the compound with respect to its therapeutic activity in the body. An example of a pharmacokinetic property that can be favorably affected is the manner in which the compound is transported across cell membranes, which in turn may directly and positively affect the absorption, distribution, biotransformation and excretion of the compound. While the route of administration of the pharmaceutical composition is important, and various anatomical, physiological and pathological factors can critically affect bioavailability, the solubility of the compound is usually dependent upon the character of the particular salt form thereof, which it utilized. One of skill in the art will appreciate that an aqueous solution of the compound will provide the most rapid absorption of the compound into the body of a subject being treated, while lipid solutions and suspensions, as well as solid dosage forms, will result in less rapid absorption of the compound.

Pharmaceutically acceptable salts include those derived from inorganic acids such as sulfuric, sulfamic, phosphoric, nitric, and the like; and the salts prepared from organic acids such as acetic, propionic, succinic, glycolic, stearic, lactic, malic, tartaric, citric, ascorbic, palmitic, maleic, hydroxymaleic, phenylacetic, glutamic, benzoic, salicyclic, sulfanilic, 2-acetoxybenzoic, fumaric, toluenesulfonic, methanesulfonic, ethane disulfonic, oxalic, isothionic, and the like. See, for example, Berge et al., “Pharmaceutical Salts”, J. Pharm. Sci. 66:1-19 (1977), the content of which is herein incorporated by reference in its entirety. Exemplary salts also include the hydrobromide, hydrochloride, sulfate, bisulfate, phosphate, nitrate, acetate, succinate, valerate, oleate, palmitate, stearate, laurate, benzoate, lactate, phosphate, tosylate, citrate, maleate, fumarate, succinate, tartrate, napthylate, mesylate, glucoheptonate, lactobionate, and laurylsulphonate salts and the like. Suitable acids which are capable of forming salts with the compounds of the disclosure include inorganic acids such as hydrochloric acid, hydrobromic acid, perchloric acid, nitric acid, thiocyanic acid, sulfuric acid, phosphoric acid, and the like; and organic acids such as 1,2-ethanedisulfonic acid, 2-hydroxyethanesulfonic acid, 2-naphthalenesulfonic acid, 3-phenylpropionic acid, 4-methylbicyclo[2.2.2]oct-2-ene-1-carboxylic acid, 4,4′-methylenebis(3-hydroxy-2-ene-1-carboxylic acid), acetic acid, anthranilic acid, benzenesulfonic acid, benzoic acid, camphorsulfonic acid, cinnamic acid, citric acid, cyclopentanepropionic acid, ethanesulfonic acid, formic acid, fumaric acid, glucoheptonic acid, gluconic acid, glutamic acid, glycolic acid, heptanoic acid, hydroxynaphthoic acid, lactic acid, lauryl sulfuric acid, maleic acid, malic acid, malonic acid, mandelic acid, methanesulfonic acid, muconic acid, naphthalene sulfonic acid, o-(4-hydroxybenzoyl)benzoic acid, oxalic acid, p-chlorobenzenesulfonic acid, propionic acid, p-toluenesulfonic acid, pyruvic acid, salicylic acid, stearic acid, succinic acid, sulfanilic acid, tartaric acid, tertiary butylacetic acid, trifluoroacetic acid, trimethylacetic acid, and the like. Suitable bases capable of forming salts with the compounds of the disclosure include inorganic bases such as sodium hydroxide, ammonium hydroxide, sodium carbonate, calcium hydroxide, potassium hydroxide and the like; and organic bases such as mono-, di- and tri-alkyl and aryl amines (e.g., triethylamine, diisopropyl amine, methyl amine, dimethyl amine, N-methylglucamine, pyridine, picoline, dicyclohexylamine, N,N′-dibezylethylenediamine, and the like), and optionally substituted ethanol-amines (e.g., ethanolamine, diethanolamine, trierhanolamine and the like).

The term “prodrug” as used herein refers to compounds that can be converted via some chemical or physiological process (e.g., enzymatic processes and metabolic hydrolysis) to compound described herein. Thus, the term “prodrug” also refers to a precursor of a biologically active compound that is pharmaceutically acceptable. A prodrug can be inactive when administered to a subject, i.e. an ester, but is converted in vivo to an active compound, for example, by hydrolysis to the free carboxylic acid or free hydroxyl. The prodrug compound often offers advantages of solubility, tissue compatibility or delayed release in an organism. The term “prodrug” is also meant to include any covalently bonded carriers, which release the active compound in vivo when such prodrug is administered to a subject. Prodrugs of an active compound, as described herein, may be prepared by modifying functional groups present in the active compound in such a way that the modifications are cleaved, either in routine manipulation or in vivo, to the parent active compound. Prodrugs include compounds wherein a hydroxy, amino or mercapto group is bonded to any group that, when the prodrug of the active compound is administered to a subject, cleaves to form a free hydroxy, free amino or free mercapto group, respectively. For example, a compound comprising a hydroxy group can be administered as an ester that is converted by hydrolysis in vivo to the hydroxy compound. Suitable esters that can be converted in vivo into hydroxy compounds include acetates, citrates, lactates, tartrates, malonates, oxalates, salicylates, propionates, succinates, fumarates, formates, benzoates, maleates, methylene-bis-b-hydroxynaphthoates, gentisates, isethionates, di-p-toluoyltartrates, methanesulfonates, ethanesulfonates, benzenesulfonates, p-toluenesulfonates, cyclohexylsulfamates, quinates, esters of amino acids, and the like. Similarly, a compound comprising an amine group can be administered as an amide, e.g., acetamide, formamide and benzamide that is converted by hydrolysis in vivo to the amine compound. See Harper, “Drug Latentiation” in Jucker, ed. Progress in Drug Research 4:221-294 (1962); Morozowich et al, “Application of Physical Organic Principles to Prodrug Design” in E. B. Roche ed. Design of Biopharmaceutical Properties through Prodrugs and Analogs, APHA Acad. Pharm. Sci. 40 (1977); Bioreversible Carriers in Drug in Drug Design, Theory and Application, E. B. Roche, ed., APHA Acad. Pharm. Sci. (1987); Design of Prodrugs, H. Bundgaard, Elsevier (1985); Wang et al. “Prodrug approaches to the improved delivery of peptide drug” in Curr. Pharm. Design. 5(4):265-287 (1999); Pauletti et al. (1997) Improvement in peptide bioavailability: Peptidomimetics and Prodrug Strategies, Adv. Drug. Delivery Rev. 27:235-256; Mizen et al. (1998) “The Use of Esters as Prodrugs for Oral Delivery of (3-Lactam antibiotics,” Pharm. Biotech. 11,:345-365; Gaignault et al. (1996) “Designing Prodrugs and Bioprecursors I. Carrier Prodrugs,” Pract. Med. Chem. 671-696; Asgharnejad, “Improving Oral Drug Transport”, in Transport Processes in Pharmaceutical Systems, G. L. Amidon, P. I. Lee and E. M. Topp, Eds., Marcell Dekker, p. 185-218 (2000); Balant et al., “Prodrugs for the improvement of drug absorption via different routes of administration”, Eur. J. Drug Metab. Pharmacokinet., 15(2): 143-53 (1990); Balimane and Sinko, “Involvement of multiple transporters in the oral absorption of nucleoside analogues”, Adv. Drug Delivery Rev., 39(1-3): 183-209 (1999); Browne, “Fosphenytoin (Cerebyx)”, Clin. Neuropharmacol. 20(1): 1-12 (1997); Bundgaard, “Bioreversible derivatization of drugs—principle and applicability to improve the therapeutic effects of drugs”, Arch. Pharm. Chemi 86(1): 1-39 (1979); Bundgaard H. “Improved drug delivery by the prodrug approach”, Controlled Drug Delivery 17: 179-96 (1987); Bundgaard H. “Prodrugs as a means to improve the delivery of peptide drugs”, Arfv. Drug Delivery Rev. 8(1): 1-38 (1992); Fleisher et al. “Improved oral drug delivery: solubility limitations overcome by the use of prodrugs”, Arfv. Drug Delivery Rev. 19(2): 115-130 (1996); Fleisher et al. “Design of prodrugs for improved gastrointestinal absorption by intestinal enzyme targeting”, Methods Enzymol. 112 (Drug Enzyme Targeting, Pt. A): 360-81, (1985); Farquhar D, et al., “Biologically Reversible Phosphate-Protective Groups”, Pharm. Sci., 72(3): 324-325 (1983); Freeman S, et al., “Bioreversible Protection for the Phospho Group: Chemical Stability and Bioactivation of Di(4-acetoxy-benzyl) Methylphosphonate with Carboxyesterase,” Chem. Soc., Chem. Commun., 875-877 (1991); Friis and Bundgaard, “Prodrugs of phosphates and phosphonates: Novel lipophilic alphaacyloxyalkyl ester derivatives of phosphate- or phosphonate containing drugs masking the negative charges of these groups”, Eur. J. Pharm. Sci. 4: 49-59 (1996); Gangwar et al., “Pro-drug, molecular structure and percutaneous delivery”, Des. Biopharm. Prop. Prodrugs Analogs, [Symp.] Meeting Date 1976, 409-21. (1977); Nathwani and Wood, “Penicillins: a current review of their clinical pharmacology and therapeutic use”, Drugs 45(6): 866-94 (1993); Sinhababu and Thakker, “Prodrugs of anticancer agents”, Adv. Drug Delivery Rev. 19(2): 241-273 (1996); Stella et al., “Prodrugs. Do they have advantages in clinical practice?”, Drugs 29(5): 455-73 (1985); Tan et al. “Development and optimization of anti-HIV nucleoside analogs and prodrugs: A review of their cellular pharmacology, structure-activity relationships and pharmacokinetics”, Adv. Drug Delivery Rev. 39(1-3): 117-151 (1999); Taylor, “Improved passive oral drug delivery via prodrugs”, Adv. Drug Delivery Rev., 19(2): 131-148 (1996); Valentino and Borchardt, “Prodrug strategies to enhance the intestinal absorption of peptides”, Drug Discovery Today 2(4): 148-155 (1997); Wiebe and Knaus, “Concepts for the design of anti-HIV nucleoside prodrugs for treating cephalic HIV infection”, Adv. Drug Delivery Rev.: 39(1-3):63-80 (1999); Waller et al., “Prodrugs”, Br. J. Clin. Pharmac. 28: 497-507 (1989), content of all of which are herein incorporated by reference in its entirety.

Methods for Treating Cancer

In various embodiments, the present invention provides a method for treating, inhibiting, reducing the severity of, preventing metastasis of and/or slowing progression of cancer in a subject in need thereof.

The methods include removing the antigen presentation process as well as T cell activation and expansion steps out of the immunosuppressive confines of the cancer patient. It enables the expansion of cytotoxic T cells using cytotoxic T cell antigens and helper antigens of cancer stem cells. These antigens can be derived from cancer stem cells and remain undefined or may use known cancer stem cell associated antigens as well as helper antigens that are defined and used to expand both cytotoxic and helper T cell antigens. Once these cells have been properly activated ex vivo, the cells are administered back to the patient for treatment.

The methods include also administering to the subject a therapeutically effective amount of an agonist of GITR. In one embodiment, the agonist is a peptide having the sequence set forth in SEQ ID NO: 1. In another embodiment, the agonist is a peptide having the sequence set forth in SEQ ID NO: 2. In a further embodiment, the methods include administering to the subject a therapeutically effective amount of a composition comprising the peptide having the sequence set forth in SEQ ID NO: 1 and SEQ ID NO: 2. In some embodiments, the GITR agonists are administered in combination with existing therapies for cancer. In some embodiments, the GITR agonists and the existing therapies are co-administered or administered sequentially. In one embodiment, the GITR agonist is administered prior to administration of existing therapies for cancer. In some embodiments, the GITR agonist is administered after administration of existing therapies for cancer. In a further embodiment, the GITR agonist is co-administered with current therapies for cancer.

In another embodiment, in addition to the ex vivo T cell activation method described herein, the invention is directed to administering to a patient suffering from cancer a sample of cells that have been enriched for the activated T-effector cells, wherein the enrichment is achieved by contacting T-effector cells with a GITR agonist such as set forth in Formula I, together with or without T-reg cells. The T-effector or T-reg cells may be autologous or allogeneic to the patient. In particular, the GITR agonist may be the RMGL171102, aka 11702 compound.

In exemplary embodiments, the cancer is B-cell lymphomas (Hodgkin's lymphomas and/or non-Hodgkins lymphomas), brain tumor, breast cancer, colon cancer, lung cancer, hepatocellular cancer, gastric cancer, pancreatic cancer, cervical cancer, ovarian cancer, liver cancer, bladder cancer, cancer of the urinary tract, thyroid cancer, renal cancer, carcinoma, melanoma, head and neck cancer, brain cancer, and prostate cancer, androgen-dependent prostate cancer and androgen-independent prostate cancer, and in particular, melanoma or lymphoma.

Methods for Treating Autoimmune Diseases

Also provided herein are methods for treating, inhibiting or reducing the severity of inflammatory diseases such as autoimmune diseases in a subject in need thereof. The methods include administering to the subject T cells that have been activated ex vivo by presenting to the T cells antigens specific for an inflammatory disease on an antigen presenting cell such as dendritic cell. Optionally, a GITR/GITRL antagonist may be added to the ex vivo T cell sample to cause greater production Treg cells as well as modified Teff cells. For instance, to treat a autoimmune disease such as multiple sclerosis, myelin associated proteins may be used as an antigen. The GITR/GITRL antagonist may be a compound of Formula II, in particular, RMGL 171104.

In some embodiments, the GITR antagonists for use in treating inflammatory disease such as autoimmune diseases are compounds having the structures set forth in Formula II.

In another embodiment, the invention is directed to administering to a patient suffering from an inflammatory disease, in particular an autoimmune disease a sample of cells that have been activated ex vivo, and enriched for T-reg cells, or wherein T-eff cells have become modified, wherein the enrichment is achieved by contacting T-effector cells with a GITR antagonist such as set forth in Formula II, together with or without T-reg cells. The T-effector or T-reg cells may be autologous or allogeneic to the patient. In particular, the GITR antagonist may be the RMGL171104, aka 11704 compound.

In some embodiments, the GITR antagonists are administered in combination with existing therapies for autoimmune diseases. In some embodiments, the GITR antagonists and the existing therapies are co-administered or simultaneously. In one embodiment, the GITR antagonist is administered prior to administration of existing therapies for autoimmune diseases. In some embodiments, the GITR antagonist is administered after administration of existing therapies for autoimmune diseases. In a further embodiment, the GITR antagonist is co-administered with current therapies for inflammatory diseases or autoimmune diseases.

In exemplary embodiments, the inflammatory disease is acute or chronic pancreatitis, and autoimmune disease is rheumatoid arthritis, osteoarthritis, asthma, dermatitis, psoriasis, cystic fibrosis, post transplantation late and chronic solid organ rejection, multiple sclerosis, systemic lupus erythematosus, Sjogren's syndrome, Hashimoto thyroiditis, polymyositis, scleroderma, Addison disease, vitiligo, pernicious anemia, glomerulonephritis and pulmonary fibrosis, inflammatory bowel diseases, autoimmune diabetes, diabetic retinopathy, rhinitis, ischemia-reperfusion injury, post-angioplasty restenosis, chronic obstructive pulmonary diseases (COPD), Grave's disease, gastrointestinal allergies, conjunctivitis, atherosclerosis, coronary artery disease, angina, cancer metastasis, small artery disease, graft-versus-host disease, or mitochondrial related syndrome, and in particular, arthritis or organ transplantation.

Combination Therapies

In exemplary embodiments, existing treatments for cancer (for use in combination GITR agonists as described herein) include but are not limited to chemotherapy, radiation therapy, hormonal therapy, surgery, immunotherapy or combinations thereof.

In some embodiments, chemotherapeutic agents may be selected from any one or more of cytotoxic antibiotics, antimetabolities, anti-mitotic agents, alkylating agents, arsenic compounds, DNA topoisomerase inhibitors, taxanes, nucleoside analogues, plant alkaloids, and toxins; and synthetic derivatives thereof. Exemplary compounds include, but are not limited to, alkylating agents: treosulfan, and trofosfamide; plant alkaloids: vinblastine, paclitaxel, docetaxol; DNA topoisomerase inhibitors: doxorubicin, epirubicin, etoposide, camptothecin, topotecan, irinotecan, teniposide, crisnatol, and mitomycin; anti-folates: methotrexate, mycophenolic acid, and hydroxyurea; pyrimidine analogs: 5-fluorouracil, doxifluridine, and cytosine arabinoside; purine analogs: mercaptopurine and thioguanine; DNA antimetabolites: 2′-deoxy-5-fluorouridine, aphidicolin glycinate, and pyrazoloimidazole; and antimitotic agents: halichondrin, colchicine, and rhizoxin. Compositions comprising one or more chemotherapeutic agents (e.g., FLAG, CHOP) may also be used. FLAG comprises fludarabine, cytosine arabinoside (Ara-C) and G-CSF. CHOP comprises cyclophosphamide, vincristine, doxorubicin, and prednisone. In another embodiments, PARP (e.g., PARP-1 and/or PARP-2) inhibitors are used and such inhibitors are well known in the art (e.g., Olaparib, ABT-888, BSI-201, BGP-15 (N-Gene Research Laboratories, Inc.); INO-1001 (Inotek Pharmaceuticals Inc.); PJ34 (Soriano et al., 2001; Pacher et al., 2002b); 3-aminobenzamide (Trevigen); 4-amino-1,8-naphthalimide; (Trevigen); 6(5H)-phenanthridinone (Trevigen); benzamide (U.S. Pat. Re. 36,397); and NU1025 (Bowman et al.).

In various embodiments, radiation therapy can be ionizing radiation. Radiation therapy can also be gamma rays, X-rays, or proton beams. Examples of radiation therapy include, but are not limited to, external-beam radiation therapy, interstitial implantation of radioisotopes (I-125, palladium, iridium), radioisotopes such as strontium-89, thoracic radiation therapy, intraperitoneal P-32 radiation therapy, and/or total abdominal and pelvic radiation therapy. For a general overview of radiation therapy, see Hellman, Chapter 16: Principles of Cancer Management: Radiation Therapy, 6th edition, 2001, DeVita et al., eds., J. B. Lippencott Company, Philadelphia. The radiation therapy can be administered as external beam radiation or tele-therapy wherein the radiation is directed from a remote source. The radiation treatment can also be administered as internal therapy or brachytherapy wherein a radioactive source is placed inside the body close to cancer cells or a tumor mass. Also encompassed is the use of photodynamic therapy comprising the administration of photosensitizers, such as hematoporphyrin and its derivatives, Vertoporfin (BPD-MA), phthalocyanine, photosensitizer Pc4, demethoxy-hypocrellin A; and 2BA-2-DMHA.

In various embodiments, immunotherapy may comprise, for example, use of cancer vaccines and/or sensitized antigen presenting cells. In some embodiments, therapies include targeting cells in the tumor microenvironment or targeting immune cells. The immunotherapy can involve passive immunity for short-term protection of a host, achieved by the administration of pre-formed antibody directed against a cancer antigen or disease antigen (e.g., administration of a monoclonal antibody, optionally linked to a chemotherapeutic agent or toxin, to a tumor antigen). Immunotherapy can also focus on using the cytotoxic lymphocyte-recognized epitopes of cancer cell lines.

In various embodiments, hormonal therapy can include, for example, hormonal agonists, hormonal antagonists (e.g., flutamide, bicalutamide, tamoxifen, raloxifene, leuprolide acetate (LUPRON), LH-RH antagonists), inhibitors of hormone biosynthesis and processing, and steroids (e.g., dexamethasone, retinoids, deltoids, betamethasone, cortisol, cortisone, prednisone, dehydrotestosterone, glucocorticoids, mineralocorticoids, estrogen, testosterone, progestins), vitamin A derivatives (e.g., all-trans retinoic acid (ATRA)); vitamin D3 analogs; antigestagens (e.g., mifepristone, onapristone), or antiandrogens (e.g., cyproterone acetate).

In some embodiments, existing therapies for autoimmune diseases include but are not limited to physical therapy, non-steroidal anti-inflammatory drugs (NSAIDs), corticosteroids, disease-modifying anti-inflammatory drugs (DMARDs), anti-cytokine therapies, inhibition of intracellular-signaling pathways, costimulation inhibition, biological inhibitors of T cell function, B-cell anergy and depletion, regulatory T cells, stem cell transplantation and/or hematopoietic stem cell transplantation.

In certain instances, the one or more GITR agonists or the GITR antagonists as described herein can be used in combination with other current or future drug therapies, because the effects of the one or more GITR agonists or the GITR antagonists as described herein alone may be less optimal by itself, and/or can be synergistic or more highly effective in combination with therapies acting on distinct pathways which interact functionally with the one or more GITR agonists or the GITR antagonists as described herein. In certain instances, conjoint administration of the one or more GITR agonists or the GITR antagonists as described herein with an additional drug therapy reduces the dose of the additional drug therapy such that it is less than the amount that achieves a therapeutic effect when used in a monotherapy.

In some embodiments, the one or more GITR agonists described herein may be combined (sequentially or simultaneously) with checkpoint inhibitors. In various embodiments, examples of immune checkpoint inhibitors for use with the GITR agonists described herein include but are not limited to anti-PD-1 antibodies such as Lambrolizumab (MK-3475), Nivolumab (BMS-936558) and Pidilizumab (CT-011), anti-PD-L1 antibodies such as MPDL3280A (RG7446), MEDI4736 and BMS-936559, anti-PD-L2 antibodies, B7-DC-Fc fusion proteins such as AMP-224, anti-CTLA-4 antibodies such as tremelimumab (CP-675,206) and ipilimumab (MDX-010), antibodies against the B7/CD28 receptor superfamily, anti-Indoleamine (2,3)-dioxygenase (IDO) antibodies, anti-IDO1 antibodies, anti-IDO2 antibodies, tryptophan, tryptophan mimetic, 1-methyl tryptophan (1-MT)), Indoximod (D-1-methyl tryptophan (D-1-MT)), L-1-methyl tryptophan (L-1-MT), TX-2274, hydroxyamidine inhibitors such as INCB024360, anti-TIM-3 antibodies, anti-LAG-3 antibodies such as BMS-986016, recombinant soluble LAG-3Ig fusion proteins that agonize MHC class II-driven dendritic cell activation such as IMP321, anti-KIR2DL1/2/3 or anti-KIR) antibodies such lirilumab (IPH2102), urelumab (BMS-663513), anti-phosphatidylserine (anti-PS) antibodies such as Bavituximab, anti-idiotype murine monoclonal antibodies against the human monoclonal antibody for N-glycolil-GM3 ganglioside such as Racotumomab (formerly known as 1E10), anti-OX40R antibodies such as IgG CD134 mAb, anti-B7-H3 antibodies such as MGA271, and small interfering (si) RNA-based cancer vaccines designed to treat cancer by silencing immune checkpoint genes. Additional information can be found in Creelan BC (Update on immune checkpoint inhibitors in lung cancer, Cancer Control. 2014 January; 21(1):80-9) and Jane de Lartigue (Another Immune Checkpoint Emerges as Anticancer Target, Published online by onclive.com, Tuesday, Sep. 24, 2013), which are incorporated herein by reference in their entirety as though fully set forth. In some embodiments, the immune checkpoint inhibitor is selected from the group consisting of an antibody against PD-1, an antibody against PD-L1, an antibody against PD-L2, an antibody against CTLA-4, an antibody against KIR, an antibody against IDO1, an antibody against IDO2, an antibody against TIM-3, an antibody against LAG-3, an antibody against OX40R, and an antibody against PS, or a combination thereof.

In various embodiments, the GITR antagonists as described herein can be used in combination with existing therapies which increase the levels of Treg cells. In exemplary embodiments, the GITR antagonist may be used in combination (sequentially or simultaneously) with TNF inhibitors including monoclonal antibodies such as infliximab (Remicade), adalimumab (Humira), certolizumab pegol (Cimzia), and golimumab (Simponi), or with a circulating receptor fusion protein such as etanercept (Enbrel).

### Kits

In various embodiments, the present invention provides a kit for treating cancers and inflammatory diseases such as autoimmune diseases. The kit comprises one or more GITR agonists (for treating cancer) and/or activated T cell, or the GITR antagonists (for treating autoimmune diseases) and instructions for use.

The exact nature of the components configured in the inventive kit depends on its intended purpose. In one embodiment, the kit is configured particularly for human subjects. In further embodiments, the kit is configured for veterinary applications, treating subjects such as, but not limited to, farm animals, domestic animals, and laboratory animals.

Instructions for use may be included in the kit. “Instructions for use” typically include a tangible expression describing the technique to be employed in using the components of the kit to effect a desired outcome, such as to treat cancers or autoimmune diseases. Optionally, the kit also contains other useful components, such as, measuring tools, diluents, buffers, pharmaceutical compositions, pharmaceutically acceptable carriers, syringes or other useful paraphernalia as will be readily recognized by those of skill in the art.

The materials or components assembled in the kit can be provided to the practitioner stored in any convenient and suitable ways that preserve their operability and utility. For example the components can be in dissolved, dehydrated, or lyophilized form; they can be provided at room, refrigerated or frozen temperatures. The components are typically contained in suitable packaging material(s). As employed herein, the phrase “packaging material” refers to one or more physical structures used to house the contents of the kit, such as inventive compositions and the like. The packaging material is constructed by well-known methods, preferably to provide a sterile, contaminant-free environment. As used herein, the term “package” refers to a suitable solid matrix or material such as glass, plastic, paper, foil, and the like, capable of holding the individual kit components. The packaging material generally has an external label which indicates the contents and/or purpose of the kit and/or its components.

Sequence Listing Free Text

As regards the use of nucleotide symbols other than a, g, c, t, they follow the convention set forth in WIPO Standard ST.25, Appendix 2, Table 1, wherein k represents t or g; n represents a, c, t or g; m represents a or c; r represents a or g; s represents c or g; w represents a or t and y represents c or t.

| Gp100 amino acid sequence | |
|---|---|
| (SEQ ID NO: 24) | |
| 1 | MDLVLKRCLL HLAVIGALLA VGATKVPRNQ DWLGVSRQLR TKAWNRQLYP EWTEAQRLDC |
| 61 | WRGGQVSLKV SNDGPTLIGA NASFSIALNF PGSQKVLPDG QVIWVNNTII NGSQVWGGQP |
| 121 | VYPQETDDAC IFPDGGPCPS GSWSQKRSFV YVWKTWGQYW QVLGGPVSGL SIGTGRAMLG |
| 181 | THTMEVTVYH RRGSRSYVPL AHSSSAFTIT DQVPFSVSVS QLRALDGGNK HFLRNQPLTF |
| 241 | ALQLHDPSGY LAEADLSYTW DFGDSSGTLI SRALVVTHTY LEPGPVTAQV VLQAAIPLTS |
| 301 | CGSSPVPGTT DGHRPTAEAP NTTAGQVPTT EVVGTTPGQA PTAEPSGTTS VQVPTTEVIS |
| 361 | TAPVQMPTAE STGMTPEKVP VSEVMGTTLA EMSTPEATGM TPAEVSIVVL SGTTAAQVTT |
| 421 | TEWVETTARE LPIPEPEGPD ASSIMSTESI TGSLGPLLDG TATLRLVKRQ VPLDCVLYRY |
| 481 | GSFSVTLDIV QGIESAEILQ AVPSGEGDAF ELTVSCQGGL PKEACMEISS PGCQPPAQRL |
| 541 | CQPVLPSPAC QLVLHQILKG GSGTYCLNVS LADTNSLAVV STQLIMPGQE AGLGQVPLIV |
| 601 | GILLVLMAVV LASLIYRRRL MKQDFSVPQL PHSSSHWLRL PRIFCSCPIG ENSPLLSGQQ |
| 661 | V |
| MAGE1 amino acid sequence | |
| (SEQ ID NO: 25) | |
| 1 | MSLEQRSLHC KPEEALEAQQ EALGLVCVQA ATSSSSPLVL GTLEEVPTAG STDPPQSPQG |
| 61 | ASAFPTTINF TRQRQPSEGS SSREEEGPST SCILESLFRA VITKKVADLV GFLLLKYRAR |
| 121 | EPVTKAEMLE SVIKNYKHCF PEIFGKASES LQLVFGIDVK EADPTGHSYV LVTCLGLSYD |
| 181 | GLLGDNQIMP KTGFLIIVLV MIAMEGGHAP EEEIWEELSV MEVYDGREHS AYGEPRKLLT |
| 241 | QDLVQEKYLE YRQVPDSDPA RYEFLWGPRA LAETSYVKVL EYVIKVSARV RFFFPSLREA |
| 301 | ALREEEEGV |
| NY-ESO-1 amino acid sequence | |
| (SEQ ID NO: 26) | |
| 1 | MQAEGRGTGG STGDADGPGG PGIPDGPGGN AGGPGEAGAT GGRGPRGAGA ARASGPGGGA |
| 61 | PRGPHGGAAS GLNGCCRCGA RGPESRLLEF YLAMPFATPM EAELARRSLA QDAPPLPVPG |
| 121 | VLLKEFTVSG NILTIRLTAA DHRQLQLSIS SCLQQLSLLM WITQCFLPVF LAQPPSGQRR |
| TRP-2 amino acid sequence | |
| (SEQ ID NO: 27) | |
| 1 | MSPLWWGFLL SCLGCKILPG AQGQFPRVCM TVDSLVNKEC CPRLGAESAN VCGSQQGRGQ |
| 61 | CTEVRADTRP WSGPYILRNQ DDRELWPRKF FHRTCKCTGN FAGYNCGDCK FGWTGPNCER |
| 121 | KKPPVIRQNI HSLSPQEREQ FLGALDLAKK RVHPDYVITT QHWLGLLGPN GTQPQFANCS |
| 181 | VYDFFVWLHY YSVRDTLLGP GRPYRAIDFS HQGPAFVTWH RYHLLCLERD LQRLIGNESF |
| 241 | ALPYWNFATG RNECDVCTDQ LFGAARPDDP TLISRNSRFS SWETVCDSLD DYNHLVTLCN |
| 301 | GTYEGLLRRN QMGRNSMKLP TLKDIRDCLS LQKFDNPPFF QNSTFSFRNA LEGFDKADGT |
| 361 | LDSQVMSLHN LVHSFLNGTN ALPHSAANDP IFVVISNRLL YNATTNILEH VRKEKATKEL |
| 421 | PSLHVLVLHS FTDAIFDEWM KRFNPPADAW PQELAPIGHN RMYNMVPFFP PVTNEELFLT |
| 481 | SDQLGYSYAI DLPVSVEETP GWPTTLLVVM GTLVALVGLF VLLAFLQYRR LRKGYTPLME |
| 541 | THLSSKRYTE EA |
| EphA2 amino acid sequence | |
| (SEQ ID NO: 28) | |
| 1 | MELQAARACF ALLWGCALAA AAAAQGKEVV LLDFAAAGGE LGWLTHPYGK GWDLMQNIMN |
| 61 | DMPIYMYSVC NVMSGDQDNW LRTNWVYRGE AERIFIELKF TVRDCNSFPG GASSCKETFN |
| 121 | LYYAESDLDY GTNFQKRLFT KIDTIAPDEI TVSSDFEARH VKLNVEERSV GPLTRKGFYL |
| 181 | AFQDIGACVA LLSVRVYYKK CPELLQGLAH FPETIAGSDA PSLATVAGTC VDHAVVPPGG |
| 241 | EEPRMHCAVD GEWLVPIGQC LCQAGYEKVE DACQACSPGF FKFEASESPC LECPEHTLPS |
| 301 | PEGATSCECE EGFFRAPQDP ASMPCTRPPS APHYLTAVGM GAKVELRWTP PQDSGGREDI |
| 361 | VYSVTCEQCW PESGECGPCE ASVRYSEPPH GLTRTSVTVS DLEPHMNYTF TVEARNGVSG |
| 421 | LVTSRSFRTA SVSINQTEPP KVRLEGRSTT SLSVSWSIPP PQQSRVWKYE VTYRKKGDSN |
| 481 | SYNVRRTEGF SVTLDDLAPD TTYLVQVQAL TQEGQGAGSK VHEFQTLSPE GSGNLAVIGG |
| 541 | VAVGVVLLLV LAGVGFFIHR RRKNQRARQS PEDVYFSKSE QLKPLKTYVD PHTYEDPNQA |
| 601 | VLKFTTEIHP SCVTRQKVIG AGEFGEVYKG MLKTSSGKKE VPVAIKTLKA GYTEKQRVDF |
| 661 | LGEAGIMGQF SHHNIIRLEG VISKYKPMMI ITEYMENGAL DKFLREKDGE FSVLQLVGML |
| 721 | RGIAAGMKYL ANMNYVHRDL AARNILVNSN LVCKVSDFGL SRVLEDDPEA TYTTSGGKIP |
| 781 | IRWTAPEAIS YRKFTSASDV WSFGIVMWEV MTYGERPYWE LSNHEVMKAI NDGFRLPTPM |
| 841 | DCPSAIYQLM MQCWQQERAR RPKFADIVSI LDKLIRAPDS LKTLADFDPR VSIRLPSTSG |
| 901 | SEGVPFRTVS EWLESIKMQQ YTEHFMAAGY TAIEKVVQMT NDDIKRIGVR LPGHQKRIAY |
| 961 | SLLGLKDQVN TVGIPI |
| HER2/NEU (HER2/ERBB2) amino acid sequence | |
| (SEQ ID NO: 29) | |
| MELAALCRWGLLLALLPPGAASTQVCTGTDMKLRLPASPETHLDMLRHLYQGCQVVQGNL | |
| ELTYLPTNASLSFLQDIQEVQGYVLIAHNQVRQVPLQRLRIVRGTQLFEDNYALAVLDNG | |
| DPLNNTTPVTGASPGGLRELQLRSLTEILKGGVLIQRNPQLCYQDTILWKDIFHKNNQLA | |
| LTLIDTNRSRACHPCSPMCKGSRCWGESSEDCQSLTRTVCAGGCARCKGPLPTDCCHEQC | |
| AAGCTGPKHSDCLACLHFNHSGICELHCPALVTYNTDTFESMPNPEGRYTFGASCVTACP | |
| YNYLSTDVGSCTLVCPLHNQEVTAEDGTQRCEKCSKPCARVCYGLGMEHLREVRAVTSAN | |
| IQEFAGCKKIFGSLAFLPESFDGDPASNTAPLQPEQLQVFETLEEITGYLYISAWPDSLP | |
| DLSVFQNLQVIRGRILHNGAYSLTLQGLGISWLGLRSLRELGSGLALIHHNTHLCFVHTV | |
| PWDQLFRNPHQALLHTANRPEDECVGEGLACHQLCARGHCWGPGPTQCVNCSQFLRGQEC | |
| VEECRVLQGLPREYVNARHCLPCHPECQPQNGSVTCFGPEADQCVACAHYKDPPFCVARC | |
| PSGVKPDLSYMPIWKFPDEEGACQPCPINCTHSCVDLDDKGCPAEQRASPLTSIISAVVG | |
| ILLVVVLGVVFGILIKRRQQKIRKYTMRRLLQETELVEPLTPSGAMPNQAQMRILKETEL | |
| RKVKVLGSGAFGTVYKGIWIPDGENVKIPVAIKVLRENTSPKANKEILDEAYVMAGVGSP | |
| YVSRLLGICLTSTVQLVTQLMPYGCLLDHVRENRGRLGSQDLLNWCMQIAKGMSYLEDVR | |
| LVHRDLAARNVLVKSPNHVKITDFGLARLLDIDETEYHADGGKVPIKWMALESILRRRFT | |
| HQSDVWSYGVTVWELMTFGAKPYDGIPAREIPDLLEKGERLPQPPICTIDVYMIMVKCWM | |
| IDSECRPRFRELVSEFSRMARDPQRFVVIQNEDLGPASPLDSTFYRSLLEDDDMGDLVDA | |
| EEYLVPQQGFFCPDPAPGAGGMVHHRHRSSSTRSGGGDLTLGLEPSEEEAPRSPLAPSEG | |
| AGSDVFDGDLGMGAAKGLQSLPTHDPSPLQRYSEDPTVPLPSETDGYVAPLTCSPQPEYV | |
| NQPDVRPQPPSPREGPLPAARPAGATLERPKTLSPGKNGVVKDVFAFGGAVENPEYLTPQ | |
| GGAAPQPHPPPAFSPAFDNLYYWDQDPPERGAPPSTFKGTPTAENPEYLGLDVPV | |
| Interleukin-13 receptor subunit alpha-2 precursor | |
| amino acid sequence | |
| (SEQ ID NO: 30) | |
| 1 | MAFVCLAIGC LYTFLISTTF GCTSSSDTEI KVNPPQDFEI VDPGYLGYLY LQWQPPLSLD |
| 61 | HFKECTVEYE LKYRNIGSET WKTIITKNLH YKDGFDLNKG IEAKIHTLLP WQCTNGSEVQ |
| 121 | SSWAETTYWI SPQGIPETKV QDMDCVYYNW QYLLCSWKPG IGVLLDTNYN LFYWYEGLDH |
| 181 | ALQCVDYIKA DGQNIGCRFP YLEASDYKDF YICVNGSSEN KPIRSSYFTF QLQNIVKPLP |
| 241 | PVYLTFTRES SCEIKLKWSI PLGPIPARCF DYEIEIREDD TTLVTATVEN ETYTLKTTNE |
| 301 | TRQLCFVVRS KVNIYCSDDG IWSEWSDKQC WEGEDLSKKT LLRFWLPFGF ILILVIFVTG |
| 361 | LLLRKPNTYP KMIPEFFCDT |
| MAGEA1 amino acid sequence | |
| (SEQ ID NO: 31) | |
| 1 | MSLEQRSLHC KPEEALEAQQ EALGLVCVQA AASSSSPLVL GTLEEVPTAG STDPPQSPQG |
| 61 | ASAFPTTINF TRQRQPSEGS SSREEEGPST SCILESLFRA VITKKVADLV GFLLLKYRAR |
| 121 | EPVTKAEMLE SVIKNYKHCF PEIFGKASES LQLVFGIDVK EADPTGHSYV LVTCLGLSYD |
| 181 | GLLGDNQIMP KTGFLIIVLV MIAMEGGHAP EEEIWEELSV MEVYDGREHS AYGEPRKLLT |
| 241 | QDLVQEKYLE YRQVPDSDPA RYEFLWGPRA LAETSYVKVL EYVIKVSARV RFFFPSLREA |
| 301 | ALREEEEGV |

All patents, patent applications, publications of patent applications, and other material, such as articles, books, specifications, publications, documents, things, and/or the like, referenced herein are hereby incorporated herein by this reference in their entirety for all purposes, excepting any prosecution file history associated with same, any of same that is inconsistent with or in conflict with the present document, or any of same that may have a limiting affect as to the broadest scope of the claims now or later associated with the present document. By way of example, should there be any inconsistency or conflict between the description, definition, and/or the use of a term associated with any of the incorporated material and that associated with the present document, the description, definition, and/or the use of the term in the present document shall prevail.

The foregoing description of various embodiments of the invention known to the applicant at this time of filing the application has been presented and is intended for the purposes of illustration and description. The present description is not intended to be exhaustive nor limit the invention to the precise form disclosed and many modifications and variations are possible in the light of the above teachings. The embodiments described serve to explain the principles of the invention and its practical application and to enable others skilled in the art to utilize the invention in various embodiments and with various modifications as are suited to the particular use contemplated. Therefore, it is intended that the invention not be limited to the particular embodiments disclosed for carrying out the invention.

### EXAMPLES

The following examples are provided to better illustrate the claimed invention and are not to be interpreted as limiting the scope of the invention. To the extent that specific materials are mentioned, it is merely for purposes of illustration and is not intended to limit the invention. One skilled in the art may develop equivalent means or reactants without the exercise of inventive capacity and without departing from the scope of the invention.

### Example 1—GITR Agonist Reduces Treg Population in Human Blood

CD4⁺CD25⁻CFES⁺ population (T effector cells) was measured in a T cell suppression assay when mixed with Treg cells in the setting of T cell activation by antigen presenting cells. Control results show T regulatory cells can inhibit 15% of T cell proliferation. GITR agonist effects dose dependent expansion of T effector cells and its activity is more effective in the presence of Tregs suggesting its impact on T effectors and Tregs. GITRL antagonist 11704 effects dose dependent retraction of T effector cells and its activity is more effective in the presence of Tregs suggesting its impact on T effectors and T regs.

### Example 2—T Cell Suppression Assay (In Vitro Human Cell)

### Example 2.1—Method

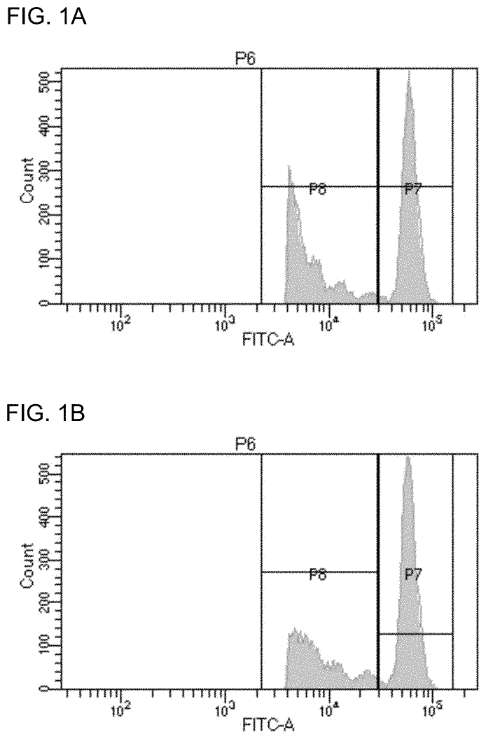

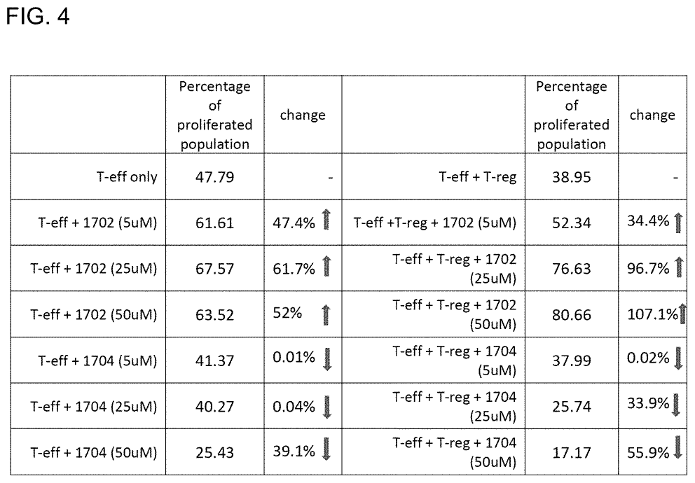

PBMC was isolated (Ficoll, GE Healthcare) from the WBC cone collected from healthy platelet donor. Cells were washed and passed through 40 um cell strainer before being stained with T cell surface antibodies. Then cells were put on cell sorter (BD FACSARIA III). Specific cell populations were collected as follows: CD4⁺CD25⁻ cells (T effector cells), CD4⁺CD25⁺CD45RA⁺CD127⁻ cells (T regulatory cells) and CD3⁻ cells (serve as Antigen Presenting Cells, APC). T effector cells were labeled with CellTrace CFSE (Invitrogen), heavily washed before cell number counting. Effector cells and T-regs were then mixed together at 1:1 ratio in culture media (RPMI 1640, 10% FBS, Pen-Strep and 1% NEAA) which enhanced with anti-CD3 (3 ug/ml) anti-CD28 (2 ug/ml) antibodies. APCs were treated with Mitomycin (50 ug/ml) for 30 minutes at 37° C., 5% CO₂ incubator, then added to culture mix (APC:T-eff 2:1) as a proliferation co-stimulator. Cell mixture was incubated at 37° C., 5% CO₂ for 6 days before being re-stained with T cell surface markers (CD4, CD25) and sent for FACS analysis (FIGS. 1A-1B, 4 and 5).

Molecule 11702 and 11704 were added to treat groups respectively at a concentration gradient of 5 uM, 25 uM and 50 uM.

### Example 2.2—Results

FACS results focus on CD4⁺CD25⁻CFES⁺ population (T effector cells). CFES fluorescence shows discrete peaks and each peak represents one generation of T lymphocyte proliferation. As T cells proliferate and replicate, CFSE peaks extend successively to left and become less fluorescent (toward negative range). The more cell population moves to left, the stronger the proliferation.

Control results show T regulatory cells can inhibit 15% of T cell proliferation.

### Example 3—Effect of RMGL171102 on T Effector Cells

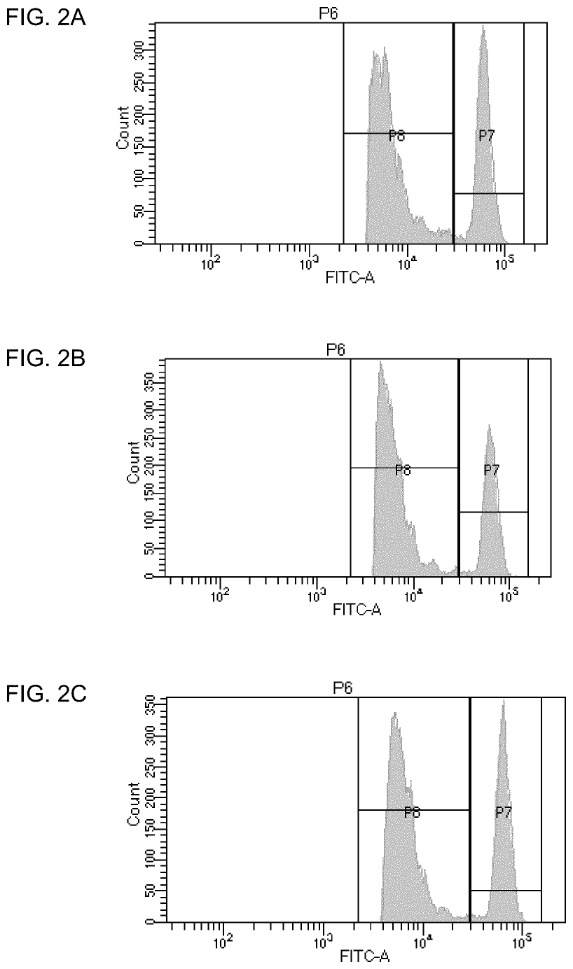

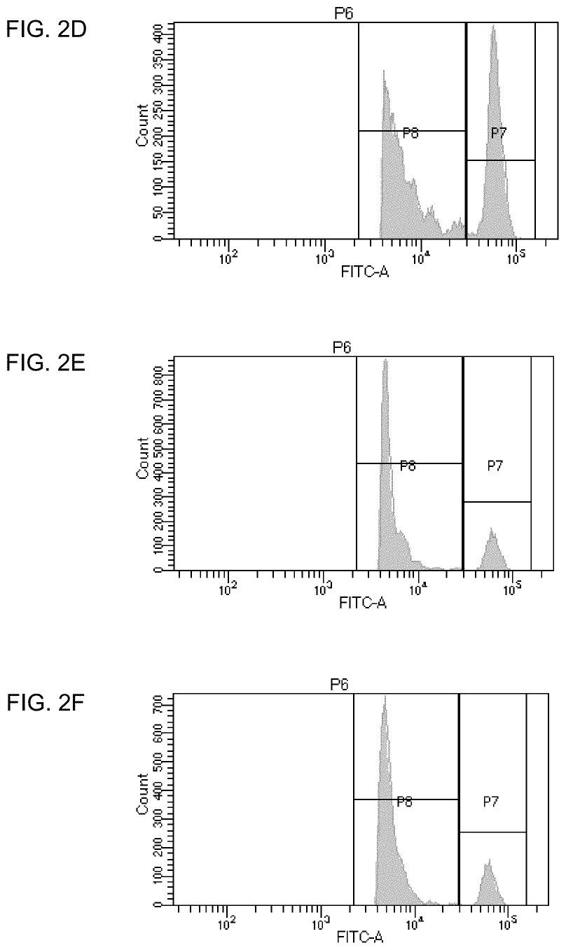

With the addition of RMGL171102(Chembridge, Inc. San Diego, ID number 5483007) to T effector (without T-reg), molecule alone shows a strong effect to accelerate T cell proliferation at all three concentrations while the most effective dose is around 25 uM (increase 61.7% of proliferated population compared to base line, 47.4% at 5 uM and 52% at 50 uM) (FIGS. 2A-2C, 4 and 5).

Within the T-reg groups (RMGL171102+T-reg), result shows much stronger stimulation of T cell expansion compared to T control group (without molecule): 34.4% increase at 5 uM, 96.7% at 25 uM and 107.1% at 50 uM (FIGS. 2D-2F, 4 and 5).

### Example 4—Effect of RMGL171104 on T-Cells

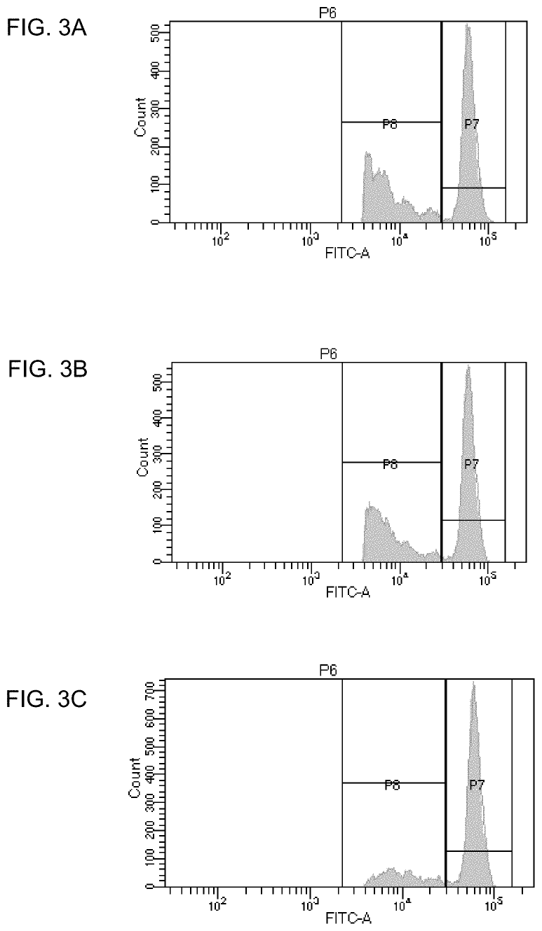

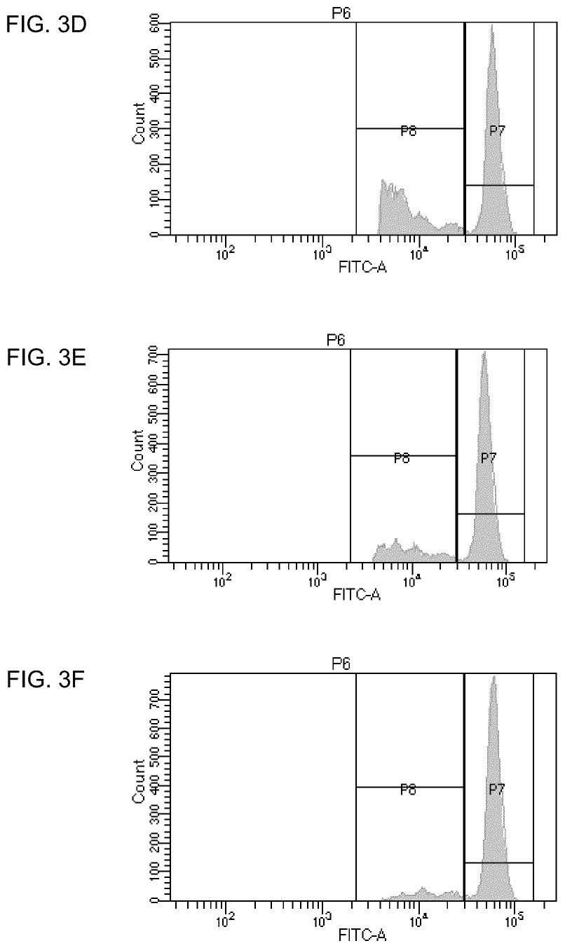

Addition of RMGL171104 alone (Chembridge, Inc. San Diego, ID number 5470140) to T effector (without T-reg) shows inhibition of T cell proliferation at concentration of 50 uM (decreases 39.1%) while no significant effect on T effector cells at 5 uM and 25 um (FIGS. 3A-3C, 4 and 5).

With T-reg involved, molecule starts to show more effective inhibition of T cell expanding at 25 uM (decreases 33.9%) and most effectively at 50 uM (decreases 55.9% of proliferation compared to base line) (FIGS. 3D-3F, 4 and 5).

### Example 5—GITR Agonist 11702 Inhibits Melanoma Growth Through Teff Proliferation and Treg Inhibition in the Tumor

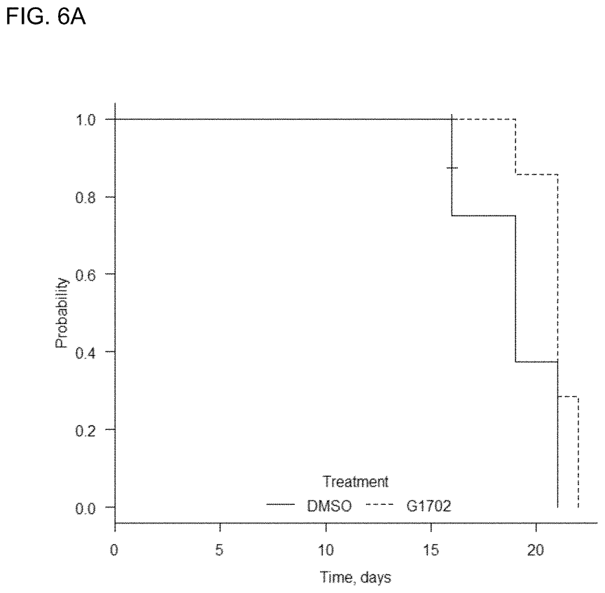

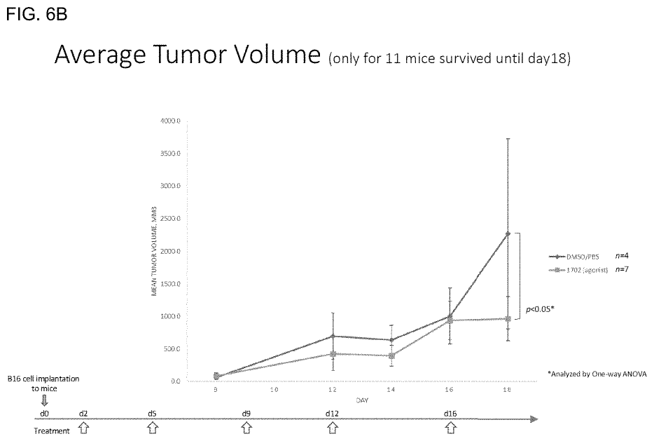

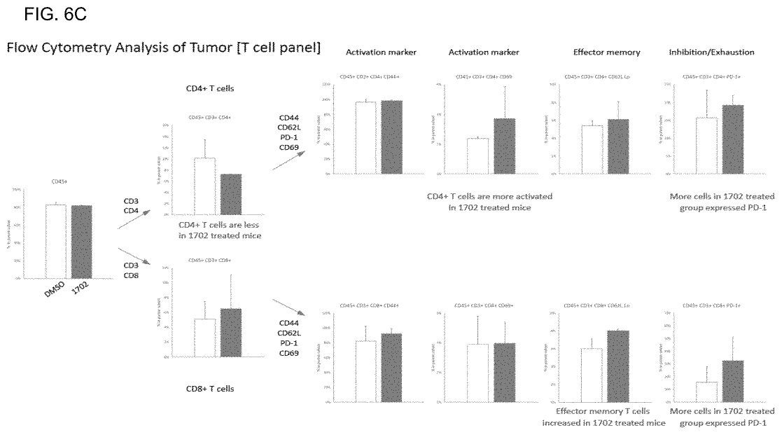

After implantation of B16 melanoma, C57 BL mice underwent treatment with 11702 GITR agonist or DMSO control. FIG. 6 shows that (A) Animals lived longer after GITR agonist intraperitoneal 30 mg/kg treatment twice per week (p=0.0333, log rank). (B) Tumor volume was inhibited in 11702 treated animals (p<0.05, Anova). (C) FACs analysis of tumor infiltrating lymphocytes demonstrated the increased presence of activated CD4+ cells and increased effector memory cytotoxic CD8+ T cells. Both of these groups showed increased PD-1 expression suggesting increased IFN gamma induced upregulation of PD-1 and invoking the potential synergy of this agent with PD-1 checkpoint blockade.

### Example 7. Methods of Activating T Cell

Procedure:

Monocytes and Lymphocytes Preparation: Elutriation

Apheresis product from patient/donor will be the source of PBMCs. Perform Elutriation (Elutra-Cell Separation System, TerumoBCT) to separate Monocytes and Lymphocytes into different fraction bags. Combine Elutra fraction 2 & 3 and collect the cells. Get rid of Red Blood Cells by co-culturing with ACK Lysing Buffer (Lonza, Walkersville, Md.). Take out a small aliquot of cells and stain with anti-CD3 antibody to confirm the purification of Lymphocytes (ideally more than 90%). Cryopreserve the Lymphocytes for future use. Centrifugate Elutra fraction 4 and 5 respectively. Wash and take out a small sample of each fraction to stain with Monocyte surface marker (CD14, CD45 and CD66). The Monocyte fraction that is used as a source of starting material for manufacturing DCs should be CD14⁺CD45⁺ population more than 60% and CD66⁺ less than 10%.

Dendritic Cell Generating: Multi-Peptides Pulsing

Monocytes will be cultured in complete DC medium along with GM-CSF and IL-4 at concentration of 3*10\hat{ }6 cells/ml. Cells will be incubated at 37° C., 5% CO2 for four days in culture bag (Cell Differentiation Bag, Miltenyi Biotec). On Day4, IFN-r and LPS are added into culture bag for DC maturation. Incubate at 37° C. and 5% CO2 overnight and on day5, peptide cocktail (each peptide with final concentration 20 ug/ml) is added into culture bag. After incubating at 37° C. and 5% CO2 for another 16-20 hours harvest, wash and count peptide-pulsed Dendritic Cells, take a small aliquot for phenotyping (CD11c⁺CD83⁺ population >60%).

T Cell Activation and Expanding: Cell Expansion Bag

Previously frozen Lymphocytes from procedure 1 are to be thawed and allow to recover in warm R10 medium (RPMI1640+10% FBS) for two hours. Re-suspend the cells with T-cell Culture Medium (TCM) enhanced by cytokine IL-4 and IL-7. T cells will be mixed with peptide-pulsed DCs (ratio 10:1) in TCM and transferred to T Cell Expansion Bags (Miltenyi Biotec.). Culture bags will be placed in 37° C., 5% CO2 incubator. After 2 days (day2), add IL-2 into culture medium to stimulate the T cell proliferation. Then IL-2 will be replenished on day5, day7 and day10. TCM in expansion bags will be changed at day5 and day10 with replenished IL-4 and IL-7. On day12, transfer/combine the medium and cells. Wash and count the cell number. At this point, samples are taken for quality control (Surface marker staining, Endotoxin test, Mycoplasma test, Environmental, Gram stain) and functioning analysis (Elispot Assay).

Alternatively T Cells can be Activated and Expanded in a G-Rex Container

Previously frozen Lymphocytes from procedure 1 are to be thawed and allow to recover in warm R10 medium (RPMI1640+10% FBS) for two hours. Re-suspend the cells with T-cell Culture Medium (TCM) enhanced by cytokine IL-4 and IL-7. T cells will be mixed with peptide-pulsed DCs (ratio 10:1) in TCM and transfer to G-Rex 100 container (Wilson Wolf Corporation). G-Rex 100 container will be placed in 37° C., 5% CO2 incubator. After 2 days (day2), add IL-2 into culture medium to stimulate the T cell proliferation. Then IL-2 will be replenished on day5, day7 and day10. TCM in G-Rex container will be changed at day5 and day10 with replenished IL-4 and IL-7. On day12, harvest cells at the end of culture. Then transfer the medium and cells into 50 ml conic tubes. Wash and count the cell number. At this point, samples are taken for quality control (Surface marker staining, Endotoxin test, Mycoplasma test, Environmental, Gram stain) and functioning analysis (Elispot Assay).

Activated T cells will be dated, labeled and cryopreserved in Liquid Nitrogen. Activated T cells will be resuspended, tested, and infused intravenously in a patient with glioblastoma.

### Example 8—General Methods and Results

The inventive steps include Step 1: Generate Dendritic Cells loaded with CSC6 lysate/peptides Step 2: Activate autologous T cells with DCs.

Cell Source: WBC Cone from Blood Donor Facility (Platelet Donor)

Monocyte Isolation Kit—for the generation of DC

T Cell Enrichment Kit—frozen down

Antigen: CSC 6 Lysate or CSC 6 Acid-Eluted Peptides

Method for DC Manufacture:

Monocytes cultured in DC medium enhanced with GM-CSF/IL-4 for 4 days.

Pulsed with CSC 6 lysate OR with peptides overnight.

Matured by adding INF-γ and LPS the following day.

DCs harvested on day 6.

Method for T Cell Activation:

TC: DC 10:1

Mixed and cultured in G-Rex (Gas Permeable Rapid Expansion) container

TC medium enhanced with IL-4/IL-7

Testing Time Points

Day 3 (cell collection and medium change)

Day 8 (cell collection and medium change, addition of IL-2)

Day 13 (Cell collected)

Results:

T Cell Surface Markers

All activation surface markers (CD137, CD69, CD45RO, CD154, HLA-DR CD62L) show up-regulation or down-regulation as expected. Both CSC 6 lysate and acid-eluted peptides create similar activation responses in T cells, but lysate shows much stronger stimulation effect. T cells expand much faster after Day8 (with addition of IL-2), and Day13 T cells show higher expression of most activation markers. (See FIGS. 7-15)

Elispot Assay (IFN-γ Secretion)

Use CSC6 lysate/peptides-loaded DCs as target/stimulator.

All stimulated groups show more than 3-fold increase of IFN-γ secretion compared to non-stimulated groups.

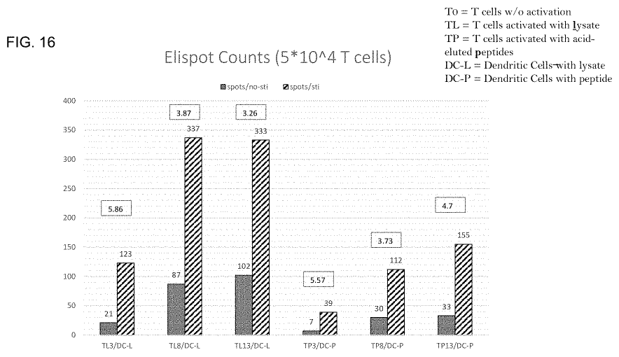

T cells stimulated by lysate for 8 & 13 days show the greatest amount of IFN-γ spots compared to other groups. (See FIG. 16)

Conclusion:

T cells can be significantly activated by autologous Dendritic Cells pulsed with CSC 6 lysate or acid-eluted peptides after 8-13 days' culturing.

CSC 6 lysate gives a greater degree of response vs. acid eluted peptides.

### Example 9. Assessment of Autoimmune Side Effect

The purpose of these experiments is to assess the autoimmune response of activated T cells by analyzing cell death in normal PHA blasts with apoptosis marker, Caspase-3.

Method:

Obtain the same PBMC, which was used for DC and T cells.

Stimulate PBMCs with PHA and cultured with IL-2.

Co-culture PHA-PBMC blasts with activated T cells.

Stain the cells with fluorophore conjugated antibodies.

Analyze by Flow Cytometry.

Results:

T cells can be significantly activated by autologous dendritic cells pulsed with CSC6 lysate or acid-eluted peptides after 8-13 days culturing.

CSC6 lysate provides a more robust response than MHC acid-eluted peptides.

No detectable autoimmunity as rate of killing of autologous cells in no higher than in controls. (See FIGS. 17-19)

While particular embodiments of the present invention have been shown and described, it will be obvious to those skilled in the art that, based upon the teachings herein, changes and modifications may be made without departing from this invention and its broader aspects and, therefore, the appended claims are to encompass within their scope all such changes and modifications as are within the true spirit and scope of this invention.

### REFERENCES

1. Wen P Y, Reardon D A, Armstrong T S, Phuphanich S, Aiken R D, Landolfi J C, Curry W T, Zhu J J, Glantz M, Peereboom D M, Markert J M, LaRocca R, O'Rourke D M, Fink K, Kim L, Gruber M, Lesser G J, Pan E, Kesari S, Muzikansky A, Pinilla C, Santos R G, Yu J S. A Randomized Double-Blind Placebo-Controlled Phase II Trial of Dendritic Cell Vaccine ICT-107 in Newly Diagnosed Patients with Glioblastoma. Clin Cancer Res. 2019; 25(19):5799-807. Epub 2019/07/18. doi: 10.1158/1078-0432.CCR-19-0261. PubMed PMID: 31320597.
2. Liu G, Ying H, Zeng G, Wheeler C J, Black K L, Yu J S. HER-2, gp100, and MAGE-1 are expressed in human glioblastoma and recognized by cytotoxic T cells. Cancer Res. 2004; 64(14):4980-6. doi: 10.1158/0008-5472.CAN-03-3504. PubMed PMID: 15256472.
3. Liu G, Yu J S, Zeng G, Yin D, Xie D, Black K L, Ying H. AIM-2: a novel tumor antigen is expressed and presented by human glioma cells. J Immunother. 2004; 27(3):220-6. doi: 10.1097/00002371-200405000-00006. PubMed PMID: 15076139.
4. Liu G, Akasaki Y, Khong H T, Wheeler C J, Das A, Black K L, Yu J S. Cytotoxic T cell targeting of TRP-2 sensitizes human malignant glioma to chemotherapy. Oncogene. 2005; 24(33):5226-34. doi: 10.1038/sj.onc.1208519. PubMed PMID: 15897911.
5. Okano F, Storkus W J, Chambers W H, Pollack I F, Okada H. Identification of a novel HLA-A*0201-restricted, cytotoxic T lymphocyte epitope in a human glioma-associated antigen, interleukin 13 receptor alpha2 chain. Clin Cancer Res. 2002; 8(9):2851-5. PubMed PMID: 12231526.
6. Pollack I F, Jakacki R I, Butterfield L H, Hamilton R L, Panigrahy A, Normolle D P, Connelly A K, Dibridge S, Mason G, Whiteside T L, Okada H. Immune responses and outcome after vaccination with glioma-associated antigen peptides and poly-ICLC in a pilot study for pediatric recurrent low-grade gliomas. Neuro Oncol. 2016; 18(8):1157-68. Epub 2016/03/15. doi: 10.1093/neuonc/now026. PubMed PMID: 26984745; PMCID: PMC4933485.
7. Pollack I F, Jakacki R I, Butterfield L H, Hamilton R L, Panigrahy A, Normolle D P, Connelly A K, Dibridge S, Mason G, Whiteside T L, Okada H. Antigen-specific immunoreactivity and clinical outcome following vaccination with glioma-associated antigen peptides in children with recurrent high-grade gliomas: results of a pilot study. J Neurooncol. 2016; 130(3):517-27. Epub 2016/09/13. doi: 10.1007/s11060-016-2245-3. PubMed PMID: 27624914; PMCID: PMC5363717.
8. Pollack I F, Jakacki R I, Butterfield L H, Hamilton R L, Panigrahy A, Potter D M, Connelly A K, Dibridge S A, Whiteside T L, Okada H. Antigen-specific immune responses and clinical outcome after vaccination with glioma-associated antigen peptides and polyinosinic-polycytidylic acid stabilized by lysine and carboxymethylcellulose in children with newly diagnosed malignant brainstem and nonbrainstem gliomas. J Clin Oncol. 2014; 32(19):2050-8. Epub 2014/06/02. doi: 10.1200/JCO.2013.54.0526. PubMed PMID: 24888813; PMCID: PMC4067943.
9. Okada H, Butterfield L H, Hamilton R L, Hoji A, Sakaki M, Ahn B J, Kohanbash G, Drappatz J, Engh J, Amankulor N, Lively M O, Chan M D, Salazar A M, Shaw E G, Potter D M, Lieberman F S. Induction of robust type-I CD8+ T-cell responses in WHO grade 2 low-grade glioma patients receiving peptide-based vaccines in combination with poly-ICLC. Clin Cancer Res. 2015; 21(2):286-94. Epub 2014/11/25. doi: 10.1158/1078-0432.CCR-14-1790. PubMed PMID: 25424847; PMCID: PMC4297523.
10. Akiyama Y, Oshita C, Kume A, Iizuka A, Miyata H, Komiyama M, Ashizawa T, Yagoto M, Abe Y, Mitsuya K, Watanabe R, Sugino T, Yamaguchi K, Nakasu Y. α-type-1 polarized dendritic cell-based vaccination in recurrent high-grade glioma: a phase I clinical trial. BMC Cancer. 2012; 12:623. Epub 2012/12/27. doi: 10.1186/1471-2407-12-623. PubMed PMID: 23270484; PMCID: PMC3541167.
11. Liu Z, Poiret T, Persson O, Meng Q, Rane L, Bartek J, Karbach J, Altmannsberger H M, Illies C, Luo X, Harvey-Peredo I, JAger E, Dodoo E, Maeurer M. NY-ESO-1- and survivin-specific T-cell responses in the peripheral blood from patients with glioma. Cancer Immunol Immunother. 2018; 67(2):237-46. Epub 2017/10/20. doi: 10.1007/s00262-017-2066-z. PubMed PMID: 29058035; PMCID: PMC5799356.
12. Kobayashi H, Wood M, Song Y, Appella E, Celis E. Defining promiscuous MHC class II helper T-cell epitopes for the HER2/neu tumor antigen. Cancer Res. 2000; 60(18):5228-36. PubMed PMID: 11016652.
13. Knutson K L, Schiffman K, Disis M L. Immunization with a HER-2/neu helper peptide vaccine generates HER-2/neu CD8 T-cell immunity in cancer patients. J Clin Invest. 2001; 107(4):477-84. doi: 10.1172/JCI 11752. PubMed PMID: 11181647; PMCID: PMC199268.
14. Osen W, Soltek S, Song M, Leuchs B, Steitz J, Tuting T, Eichmuller S B, Nguyen X D, Schadendorf D, Paschen A. Screening of human tumor antigens for CD4 T cell epitopes by combination of HLA-transgenic mice, recombinant adenovirus and antigen peptide libraries. PLoS One. 2010; 5(11):e14137. Epub 2010/11/30. doi: 10.1371/journal.pone.0014137. PubMed PMID: 21152437; PMCID: PMC2994730.
15. Kobayashi H, Lu J, Celis E. Identification of helper T-cell epitopes that encompass or lie proximal to cytotoxic T-cell epitopes in the gp100 melanoma tumor antigen. Cancer Res. 2001; 61(20):7577-84. PubMed PMID: 11606397.
16. Tatsumi T, Herrem C J, Olson W C, Finke J H, Bukowski R M, Kinch M S, Ranieri E, Storkus W J. Disease stage variation in CD4+ and CD8+ T-cell reactivity to the receptor tyrosine kinase EphA2 in patients with renal cell carcinoma. Cancer Res. 2003; 63(15):4481-9. PubMed PMID: 12907621.
17. Hatano M, Eguchi J, Tatsumi T, Kuwashima N, Dusak J E, Kinch M S, Pollack I F, Hamilton R L, Storkus W J, Okada H. EphA2 as a glioma-associated antigen: a novel target for glioma vaccines. Neoplasia. 2005; 7(8):717-22. doi: 10.1593/neo.05277. PubMed PMID: 16207473; PMCID: PMC1501889.
18. Odunsi K, Qian F, Matsuzaki J, Mhawech-Fauceglia P, Andrews C, Hoffman E W, Pan L, Ritter G, Villella J, Thomas B, Rodabaugh K, Lele S, Shrikant P, Old L J, Gnjatic S. Vaccination with an NY-ESO-1 peptide of HLA class I/II specificities induces integrated humoral and T cell responses in ovarian cancer. Proc Natl Acad Sci USA. 2007; 104(31):12837-42. Epub 2007/07/25. doi: 10.1073/pnas.0703342104. PubMed PMID: 17652518; PMCID: PMC1937553.
19. Matsuzaki J, Qian F, Luescher I, Lele S, Ritter G, Shrikant P A, Gnjatic S, Old L J, Odunsi K. Recognition of naturally processed and ovarian cancer reactive CD8+ T cell epitopes within a promiscuous HLA class II T-helper region of NY-ESO-1. Cancer Immunol Immunother. 2008; 57(8):1185-95. Epub 2008/02/06. doi: 10.1007/s00262-008-0450-4. PubMed PMID: 18253733.
20. Liu G, Ying H, Zeng G et al. HER-2, gp100, and MAGE-1 are expressed in human glioblastoma and recognized by cytotoxic T cells. Cancer Res. 2004; 64:4980-4986.
21. Harada M, Li Y F, El-Gamil M et al. Melanoma-Reactive CD8+ T cells recognize a novel tumor antigen expressed in a wide variety of tumor types. J Immunother. 2001; 24:323-333.
22. Liu G, Yu J S, Zeng G et al. AIM-2: a novel tumor antigen is expressed and presented by human glioma cells. J Immunother. 2004; 27:220-226.
23. Liu G, Khong H T, Wheeler C J et al. Molecular and functional analysis of tyrosinase-related protein (TRP)-2 as a cytotoxic T lymphocyte target in patients with malignant glioma. J Immunother. 2003; 26:301-312.
24. Mintz A, Gibo D M, Slagle-Webb B, Christensen N D, Debinski W. IL-13Ralpha2 is a glioma-restricted receptor for interleukin-13. Neoplasia. 2002; 4:388-399.
25. Okano F, Storkus W J, Chambers W H, Pollack I F, Okada H. Identification of a novel HLA-A*0201-restricted, cytotoxic T lymphocyte epitope in a human glioma-associated antigen, interleukin 13 receptor alpha2 chain. Clin. Cancer Res. 2002; 8:2851-2855.
26. Okano F, Storkus W J, Chambers W H, Pollack I F, Okada H. Identification of a novel HLA-A*0201-restricted, cytotoxic T lymphocyte epitope in a human glioma-associated antigen, interleukin 13 receptor alpha2 chain. Clin. Cancer Res. 2002; 8:2851-2855.

1. A method for treating cancer in a subject in need thereof comprising administering to the subject a therapeutically effective amount of T cells that have been activated ex vivo with an antigen presenting cell.

2. The method of claim 1, wherein the antigen presenting cell is dendritic cell.

3. The method of claim 1, wherein the antigen presenting cell bears cancer stem cell antigen.

4. The method of claim 1, wherein the antigen is a polypeptide of gp100, MAGE1, NY-ESO-1, TRP-2, EphA2, AIM2, HER2/neu, IL-13Ra2, or MAGE-A1, or a combination thereof.

5. The method of claim 4, wherein the polypeptide is about 8 to about 20 amino acids long, more preferably, about 8 to about 13 amino acids long, wherein the polypeptide is an epitope for activation of T cells.

6. The method of claim 5, wherein

| | for gp100, the polypeptide is |
|---|---|
| (SEQ ID NO: 6) | |
| | IMDQVPFSV; |
| | for MAGE1, the polypeptide is |
| (SEQ ID NO: 7) | |
| | EADPTGHSY; |
| | for NY-ESO-1, the polypeptide is |
| (SEQ ID NO: 8) | |
| | SLLMWITQC; |
| | for TRP-2, the polypeptide is |
| (SEQ ID NO: 9) | |
| | SVYDFFVWL; |
| | for EphA2, the polypeptide is |
| (SEQ ID NO: 10) | |
| | TLADFDPRV; |
| | for AIM2, the polypeptide is |
| (SEQ ID NO: 11) | |
| | RSDSGQQARY; |
| | for HER2/neu, the polypeptide is |
| (SEQ ID NO: 12) | |
| | VMAGVGSPYV; |
| | for IL-13Ra2, the polypeptide is |
| (SEQ ID NO: 13) | |
| | WLPFGFIL; |
| | and |
| | for MAGE-A1, the polypeptide is |
| (SEQ ID NO: 14) | |
| | KVLEYVIKV. |

7. The method of claim 1, comprising helper antigen, wherein the helper is a polypeptide of antigen gp100, NY-ESO-1, TRP-2, EphA2, HER2/neu, or MAGE-A1, or a combination thereof.

8. The method of claim 7, wherein the polypeptide is about 8 to about 30 amino acids, preferably 8 to about 20, or about 8 to about 12 amino acids, wherein the polypeptide is an epitope for activation of T cells.

9. The method of claim 8, wherein

| | for gp100, the polypeptide is |
|---|---|
| (SEQ ID NO: 15) | |
| | SLAVVSTQLIMPGQE; |
| | for NY-ESO-1, the polypeptide is |
| (SEQ ID NO: 16) | |
| | PGVLLKEFTVSGNILTIRLTAADHR; |
| | for TRP-2, the polypeptide is |
| (SEQ ID NO: 17) | |
| | QCTEVRADTRPWSGP |
| | or |
| (SEQ ID NO: 18) | |
| | KKRVHPDYVITTQHWL; |
| | for EphA2, the polypeptide is |
| (SEQ ID NO: 19) | |
| | EAGIMGQFSHHNIIR; |
| | and |
| | for HER2/neu, the polypeptide is |
| (SEQ ID NO: 20) | |
| | KVPIKWMALESILRRRF, |
| (SEQ ID NO: 21) | |
| | KIFGSLAFLPESFDGDPA, |
| (SEQ ID NO: 22) | |
| | RRLLQETELVEPLTPS, |
| | or |
| (SEQ ID NO: 23) | |
| | ELVSEFSRMARDPQ. |

10. The method of claim 1, further comprising contacting the T cells with GITR/GITRL agonist.

11. The method of claim 10, wherein the GITR/GITRL agonist is represented by a compound of Formula I 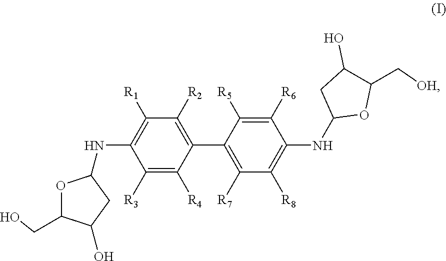
or a pharmaceutically acceptable salt, ester or prodrug thereof;
wherein:
R₁ is hydrogen or an optionally substituted substituent;
R₂ is hydrogen or an optionally substituted substituent;
R₃ is hydrogen or an optionally substituted substituent;
R₄ is hydrogen or an optionally substituted substituent;
R₅ is hydrogen or an optionally substituted substituent;
R₆ is hydrogen or an optionally substituted substituent;
R₇ is hydrogen or an optionally substituted substituent; and
R₈ is hydrogen or an optionally substituted substituent;
wherein optionally any two or more of R₁, R₂, R₃, R₄, R₅, R₆, R₇, or R₈ may be joined together to form one or more rings.

12. The method of claim 11, wherein the GITR/GITRL agonist compound is

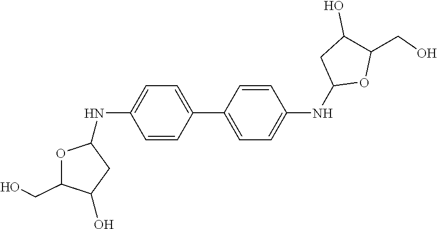 [SMILES: OCC1OC(Nc2ccc(-c3ccc(NC4CC(O)C(CO)O4)cc3)cc2)CC1O]

13. The method of claim 11, wherein the GITR/GITRL agonist is represented by a peptide having the sequence set forth in SEQ ID NO:1 or 2 or a variant, derivative or functional equivalent thereof.

14. The method of claim 1, further comprising administering existing therapies for cancer to the subject either co-administered or sequentially.

15. The method of claim 1, wherein the cancer is T-cell/B-cell lymphomas (Hodgkin's lymphomas and/or non-Hodgkins lymphomas), brain tumor, breast cancer, colon cancer, lung cancer, hepatocellular cancer, gastric cancer, pancreatic cancer, cervical cancer, ovarian cancer, liver cancer, bladder cancer, cancer of the urinary tract, thyroid cancer, renal cancer, carcinoma, skin cancer, head and neck cancer, brain cancer, and prostate cancer, androgen-dependent prostate cancer and androgen-independent prostate cancer.

16. The method of claim 15, wherein the brain cancer is glioblastoma.

17. A method for treating cancer in a subject in need thereof comprising administering to the subject a therapeutically effective amount of a sample of T-eff cells and/or cytotoxic T lymphocytes (CTL) cells that have been activated ex vivo, and enriched or expanded, wherein the T-eff cells and/or CTL cells are enriched or expanded by contacting the T-eff cells and/or CTL with a GITR/GITRL agonist with or without the presence of T-reg cells.

18. The method of claim 17, wherein the CTL, T-eff or T-reg cells are autologous relative to the subject.

19. The method of claim 17, wherein the CTL, T-eff or T-reg cells are allogeneic relative to the subject.

20. The method of claim 17, wherein the GITR/GITRL agonist is a compound of Formula I: 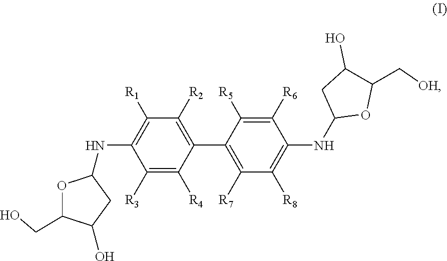
or a pharmaceutically acceptable salt, ester or prodrug thereof;
wherein:
R₁ is hydrogen or an optionally substituted substituent;
R₂ is hydrogen or an optionally substituted substituent;
R₃ is hydrogen or an optionally substituted substituent;
R₄ is hydrogen or an optionally substituted substituent;
R₅ is hydrogen or an optionally substituted substituent;
R₆ is hydrogen or an optionally substituted substituent;
R₇ is hydrogen or an optionally substituted substituent; and
R₈ is hydrogen or an optionally substituted substituent;
wherein optionally any two or more of R₁, R₂, R₃, R₄, R₅, R₆, R₇, or R₈ may be joined together to form one or more rings.

21. The method of claim 20, wherein the compound of Formula I is

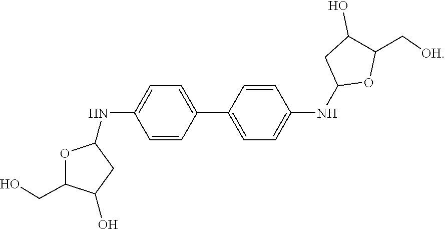 [SMILES: OCC1OC(Nc2ccc(-c3ccc(NC4CC(O)C(CO)O4)cc3)cc2)CC1O]

22. A method of providing activated T-cells, comprising activating T-cells by contacting ex vivo T cells with antigen bearing antigen presenting cells, and enriching or expanding T-eff or CTL cells comprising contacting T-eff or CTL cells with a GITR/GITRL agonist with or without the presence of T-reg cells.

23. The method of claim 22, wherein T-reg cells are present.

24. The method of claim 22, wherein GITR/GITRL agonist is a compound of Formula I: 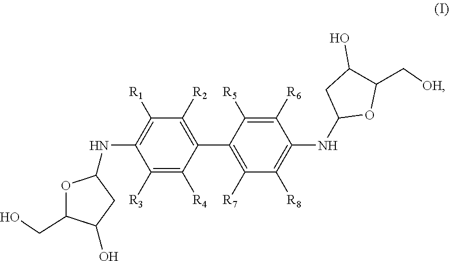
or a pharmaceutically acceptable salt, ester or prodrug thereof;
wherein:
R₁ is hydrogen or an optionally substituted substituent;
R₂ is hydrogen or an optionally substituted substituent;
R₃ is hydrogen or an optionally substituted substituent;
R₄ is hydrogen or an optionally substituted substituent;
R₅ is hydrogen or an optionally substituted substituent;
R₆ is hydrogen or an optionally substituted substituent;
R₇ is hydrogen or an optionally substituted substituent; and
R₈ is hydrogen or an optionally substituted substituent;
wherein optionally any two or more of R₁, R₂, R₃, R₄, R₅, R₆, R₇, or R₈ may be joined together to form one or more rings.

25. The method of claim 20, wherein the compound of Formula I is

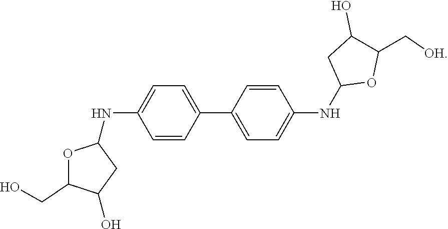 [SMILES: OCC1OC(Nc2ccc(-c3ccc(NC4CC(O)C(CO)O4)cc3)cc2)CC1O]

26. The method of claim 23, wherein the T-eff or CTL and T-reg cells are present in a starting ratio of about 1:1.

27.-57. (canceled)


SEQUENCE LISTING (31 sequences)

SEQ ID NO: 1 - protein, 397 aa, Artificial Sequence
  Agonist of GITR
GAMASQLETAKEPCMAKFGPLPSKWQMASSEPPCVNKVSDWKLEILQNGLYLIYGQVAPNANYNDVAPFEVRLYKNKDMIQTLTNKSKIQNVGGTYELHVGDTIDLIFNSEHQVLKNNTYWGIILLANPQFISGSGSGSGSNKEPCMAKFGPLPSKWQMASSEPPCVNKVSDWKLEILQNGLYLIYGQVAPNANYNDVAPFEVRLYKNKDMIQTLTNKSKIQNVGGTYELHVGDTIDLIFNSEHQVLKNNTYWGIILLANPQFISGSGSGSGSNKEPCMAKFGPLPSKWQMASSEPPCVNKVSDWKLEILQNGLYLIYGQVAPNANYNDVAPFEVRLYKNKDMIQTLTNKSKIQNVGGTYELHVGDTIDLIFNSEHQVLKNNTYWGIILLANPQFIS

SEQ ID NO: 2 - protein, 672 aa, Artificial Sequence
  Agonist of GITR
TGGRNSIRYSELAPLFDTTRVYLVDNKSTDVASLNYQNDHSNFLTTVIQNNDYSPGEASTQTINLDDRSHWGGDLKTILHTNMPNVNEFMFTNKFKARVMVSRSLTKDKQVELKYEWVEFTLPEGNYSETMTIDLMNNAIVEHYLKVGRQNGVLESDIGVKFDTRNFRLGFDPVTGLVMPGVYTNEAFHPDIILLPGCGVDFTHSRLSNLLGIRKRQPFQEGFRITYDDLEGGNIPALLDVDAYQASLKDDTEQGGDGAGGGNNSGSGAEENSNAAAAAMQPVEDMNDHAINGSTFATRAEEKRAEAEAAAEAAAPAAQPEVEKPQKKPVIKPLTEDSKKRSYNLISNDSTFTQYRSWYLAYNYGDPQTGIRSWTLLCTPDVTCGSEQVYWSLPDMMQDPVTFRSTSQISNFPVVGAELLPVHSKSFYNDQAVYSQLIRQFTSLTHVFNRFPENQILARPPAPTITTVSENVPALTDHGTLPLRNSIGGVQRVTITDARRRTCPYVYKALGIVSPRVLSSRTGSGSGSGSNGAMASQLETAKEPCMAKFGPLPSKWQMASSEPPCVNKVSDWKLEILQNGLYLIYGQVAPNANYNDVAPFEVRLYKNKDMIQTLTNKSKIQNVGGTYELHVGDTIDLIFNSEHQVLKNNTYWGIILLANPQFISGSHHHHHH

SEQ ID NO: 3 - protein, 199 aa, Homo sapiens
MTLHPSPITCEFLFSTALISPKMCLSHLENMPLSHSRTQGAQRSSWKLWLFCSIVMLLFLCSFSWLIFIFLQLETAKEPCMAKFGPLPSKWQMASSEPPCVNKVSDWKLEILQNGLYLIYGQVAPNANYNDVAPFEVRLYKNKDMIQTLTNKSKIQNVGGTYELHVGDTIDLIFNSEHQVLKNNTYWGIILLANPQFIS

SEQ ID NO: 4 - protein, 123 aa, Artificial Sequence
  Agonist of GITR
KEPCMAKFGPLPSKWQMASSEPPCVNKVSDWKLEILQNGLYLIYGQVAPNANYNDVAPFEVRLYKNKDMIQTLTNKSKIQNVGGTYELHVGDTIDLIFNSEHQVLKNNTYWGIILLANPQFIS

SEQ ID NO: 5 - protein, 8 aa, Artificial Sequence
  linker sequence
GSGSGSGS

SEQ ID NO: 6 - protein, 9 aa, Artificial Sequence
  gp100 antigen polypeptide
IMDQVPFSV

SEQ ID NO: 7 - protein, 9 aa, Artificial Sequence
  MAGE1 antigen polypeptide
EADPTGHSY

SEQ ID NO: 8 - protein, 9 aa, Artificial Sequence
  NY-ESO-1 antgen polypeptide
SLLMWITQC

SEQ ID NO: 9 - protein, 9 aa, Artificial Sequence
  TRP-2 antigen polypeptide
SVYDFFVWL

SEQ ID NO: 10 - protein, 9 aa, Artificial Sequence
  EphA2 antigenic polypeptide
TLADFDPRV

SEQ ID NO: 11 - protein, 10 aa, Artificial Sequence
  AIM2 antigenic polypeptide
RSDSGQQARY

SEQ ID NO: 12 - protein, 10 aa, Artificial Sequence
  HER2/neu antigenic polypeptide
VMAGVGSPYV

SEQ ID NO: 13 - protein, 8 aa, Artificial Sequence
  IL-13Ra2 antigenic polypeptide
WLPFGFIL

SEQ ID NO: 14 - protein, 9 aa, Artificial Sequence
  MAGE-A1 antigenic polypeptide
KVLEYVIKV

SEQ ID NO: 15 - protein, 15 aa, Artificial Sequence
  gp100 antigenic polypeptide
SLAVVSTQLIMPGQE

SEQ ID NO: 16 - protein, 25 aa, Artificial Sequence
  NY-ESO-1 antigenic polypeptide
PGVLLKEFTVSGNILTIRLTAADHR

SEQ ID NO: 17 - protein, 15 aa, Artificial Sequence
  TRP-2 antigenic polypeptide
QCTEVRADTRPWSGP

SEQ ID NO: 18 - protein, 16 aa, Artificial Sequence
  TRP-2 antigenic polypeptide
KKRVHPDYVITTQHWL

SEQ ID NO: 19 - protein, 15 aa, Artificial Sequence
  EphA2 antigenic polypeptide
EAGIMGQFSHHNIIR

SEQ ID NO: 20 - protein, 17 aa, Artificial Sequence
  HER2/neu antigenic polypeptide
KVPIKWMALESILRRRF

SEQ ID NO: 21 - protein, 18 aa, Artificial Sequence
  HER2/neu antigenic polypeptide
KIFGSLAFLPESFDGDPA

SEQ ID NO: 22 - protein, 16 aa, Artificial Sequence
  HER2/neu antigenic polypeptide
RRLLQETELVEPLTPS

SEQ ID NO: 23 - protein, 14 aa, Artificial Sequence
  HER2/neu antigenic polypeptide
ELVSEFSRMARDPQ

SEQ ID NO: 24 - protein, 661 aa, Homo sapiens
MDLVLKRCLLHLAVIGALLAVGATKVPRNQDWLGVSRQLRTKAWNRQLYPEWTEAQRLDCWRGGQVSLKVSNDGPTLIGANASFSIALNFPGSQKVLPDGQVIWVNNTIINGSQVWGGQPVYPQETDDACIFPDGGPCPSGSWSQKRSFVYVWKTWGQYWQVLGGPVSGLSIGTGRAMLGTHTMEVTVYHRRGSRSYVPLAHSSSAFTITDQVPFSVSVSQLRALDGGNKHFLRNQPLTFALQLHDPSGYLAEADLSYTWDFGDSSGTLISRALVVTHTYLEPGPVTAQVVLQAAIPLTSCGSSPVPGTTDGHRPTAEAPNTTAGQVPTTEVVGTTPGQAPTAEPSGTTSVQVPTTEVISTAPVQMPTAESTGMTPEKVPVSEVMGTTLAEMSTPEATGMTPAEVSIVVLSGTTAAQVTTTEWVETTARELPIPEPEGPDASSIMSTESITGSLGPLLDGTATLRLVKRQVPLDCVLYRYGSFSVTLDIVQGIESAEILQAVPSGEGDAFELTVSCQGGLPKEACMEISSPGCQPPAQRLCQPVLPSPACQLVLHQILKGGSGTYCLNVSLADTNSLAVVSTQLIMPGQEAGLGQVPLIVGILLVLMAVVLASLIYRRRLMKQDFSVPQLPHSSSHWLRLPRIFCSCPIGENSPLLSGQQV

SEQ ID NO: 25 - protein, 309 aa, Homo sapiens
MSLEQRSLHCKPEEALEAQQEALGLVCVQAATSSSSPLVLGTLEEVPTAGSTDPPQSPQGASAFPTTINFTRQRQPSEGSSSREEEGPSTSCILESLFRAVITKKVADLVGFLLLKYRAREPVTKAEMLESVIKNYKHCFPEIFGKASESLQLVFGIDVKEADPTGHSYVLVTCLGLSYDGLLGDNQIMPKTGFLIIVLVMIAMEGGHAPEEEIWEELSVMEVYDGREHSAYGEPRKLLTQDLVQEKYLEYRQVPDSDPARYEFLWGPRALAETSYVKVLEYVIKVSARVRFFFPSLREAALREEEEGV

SEQ ID NO: 26 - protein, 180 aa, Homo sapiens
MQAEGRGTGGSTGDADGPGGPGIPDGPGGNAGGPGEAGATGGRGPRGAGAARASGPGGGAPRGPHGGAASGLNGCCRCGARGPESRLLEFYLAMPFATPMEAELARRSLAQDAPPLPVPGVLLKEFTVSGNILTIRLTAADHRQLQLSISSCLQQLSLLMWITQCFLPVFLAQPPSGQRR

SEQ ID NO: 27 - protein, 552 aa, Homo sapiens
MSPLWWGFLLSCLGCKILPGAQGQFPRVCMTVDSLVNKECCPRLGAESANVCGSQQGRGQCTEVRADTRPWSGPYILRNQDDRELWPRKFFHRTCKCTGNFAGYNCGDCKFGWTGPNCERKKPPVIRQNIHSLSPQEREQFLGALDLAKKRVHPDYVITTQHWLGLLGPNGTQPQFANCSVYDFFVWLHYYSVRDTLLGPGRPYRAIDFSHQGPAFVTWHRYHLLCLERDLQRLIGNESFALPYWNFATGRNECDVCTDQLFGAARPDDPTLISRNSRFSSWETVCDSLDDYNHLVTLCNGTYEGLLRRNQMGRNSMKLPTLKDIRDCLSLQKFDNPPFFQNSTFSFRNALEGFDKADGTLDSQVMSLHNLVHSFLNGTNALPHSAANDPIFVVISNRLLYNATTNILEHVRKEKATKELPSLHVLVLHSFTDAIFDEWMKRFNPPADAWPQELAPIGHNRMYNMVPFFPPVTNEELFLTSDQLGYSYAIDLPVSVEETPGWPTTLLVVMGTLVALVGLFVLLAFLQYRRLRKGYTPLMETHLSSKRYTEEA

SEQ ID NO: 28 - protein, 976 aa, Homo sapiens
MELQAARACFALLWGCALAAAAAAQGKEVVLLDFAAAGGELGWLTHPYGKGWDLMQNIMNDMPIYMYSVCNVMSGDQDNWLRTNWVYRGEAERIFIELKFTVRDCNSFPGGASSCKETFNLYYAESDLDYGTNFQKRLFTKIDTIAPDEITVSSDFEARHVKLNVEERSVGPLTRKGFYLAFQDIGACVALLSVRVYYKKCPELLQGLAHFPETIAGSDAPSLATVAGTCVDHAVVPPGGEEPRMHCAVDGEWLVPIGQCLCQAGYEKVEDACQACSPGFFKFEASESPCLECPEHTLPSPEGATSCECEEGFFRAPQDPASMPCTRPPSAPHYLTAVGMGAKVELRWTPPQDSGGREDIVYSVTCEQCWPESGECGPCEASVRYSEPPHGLTRTSVTVSDLEPHMNYTFTVEARNGVSGLVTSRSFRTASVSINQTEPPKVRLEGRSTTSLSVSWSIPPPQQSRVWKYEVTYRKKGDSNSYNVRRTEGFSVTLDDLAPDTTYLVQVQALTQEGQGAGSKVHEFQTLSPEGSGNLAVIGGVAVGVVLLLVLAGVGFFIHRRRKNQRARQSPEDVYFSKSEQLKPLKTYVDPHTYEDPNQAVLKFTTEIHPSCVTRQKVIGAGEFGEVYKGMLKTSSGKKEVPVAIKTLKAGYTEKQRVDFLGEAGIMGQFSHHNIIRLEGVISKYKPMMIITEYMENGALDKFLREKDGEFSVLQLVGMLRGIAAGMKYLANMNYVHRDLAARNILVNSNLVCKVSDFGLSRVLEDDPEATYTTSGGKIPIRWTAPEAISYRKFTSASDVWSFGIVMWEVMTYGERPYWELSNHEVMKAINDGFRLPTPMDCPSAIYQLMMQCWQQERARRPKFADIVSILDKLIRAPDSLKTLADFDPRVSIRLPSTSGSEGVPFRTVSEWLESIKMQQYTEHFMAAGYTAIEKVVQMTNDDIKRIGVRLPGHQKRIAYSLLGLKDQVNTVGIPI

SEQ ID NO: 29 - protein, 1255 aa, Homo sapiens [sequence omitted]

SEQ ID NO: 30 - protein, 380 aa, Homo sapiens
MAFVCLAIGCLYTFLISTTFGCTSSSDTEIKVNPPQDFEIVDPGYLGYLYLQWQPPLSLDHFKECTVEYELKYRNIGSETWKTIITKNLHYKDGFDLNKGIEAKIHTLLPWQCTNGSEVQSSWAETTYWISPQGIPETKVQDMDCVYYNWQYLLCSWKPGIGVLLDTNYNLFYWYEGLDHALQCVDYIKADGQNIGCRFPYLEASDYKDFYICVNGSSENKPIRSSYFTFQLQNIVKPLPPVYLTFTRESSCEIKLKWSIPLGPIPARCFDYEIEIREDDTTLVTATVENETYTLKTTNETRQLCFVVRSKVNIYCSDDGIWSEWSDKQCWEGEDLSKKTLLRFWLPFGFILILVIFVTGLLLRKPNTYPKMIPEFFCDT

SEQ ID NO: 31 - protein, 309 aa, Homo sapiens
MSLEQRSLHCKPEEALEAQQEALGLVCVQAAASSSSPLVLGTLEEVPTAGSTDPPQSPQGASAFPTTINFTRQRQPSEGSSSREEEGPSTSCILESLFRAVITKKVADLVGFLLLKYRAREPVTKAEMLESVIKNYKHCFPEIFGKASESLQLVFGIDVKEADPTGHSYVLVTCLGLSYDGLLGDNQIMPKTGFLIIVLVMIAMEGGHAPEEEIWEELSVMEVYDGREHSAYGEPRKLLTQDLVQEKYLEYRQVPDSDPARYEFLWGPRALAETSYVKVLEYVIKVSARVRFFFPSLREAALREEEEGV